(19) United States
(12) Patent Application Publication
(10) Pub. No.: US 2011/0177109 A1
(43) Pub. Date: Jul. 21, 2011

(54) ANTIGENIC TAU PEPTIDES AND USES THEREOF

(75) Inventors: George Joseph Smith, III, San Diego, CA (US); Kenneth Nelson Wills, Carlsbad, CA (US); Jeff Xianchao Zhu, San Diego, CA (US)

(73) Assignee: PFIZER VACCINES LLC, New York, NY (US)

(21) Appl. No.: 12/846,719

(22) Filed: Jul. 29, 2010

Related U.S. Application Data
(60) Provisional application No. 61/229,860, filed on Jul. 30, 2009.

(51) Int. Cl. A61K 39/00; C07K 7/06; A61P 25/28; C07K 7/08; C07K 14/435

(52) U.S. Cl. 424/185.1; 530/329; 530/328; 530/327; 530/326; 530/325; 530/324

(57) ABSTRACT

The present disclosure relates to novel immunogens and compositions comprising an antigenic tau peptide, preferably linked to an immunogenic carrier for use in the treatment of tau-related neurological disorders. The disclosure further relates to methods for production of these immunogens and compositions and their use in medicine.

This application claims priority to U.S. Provisional Application No. 61/229,860 filed on Jul. 30, 2009, which is incorporated herein by reference in its entirety.

### REFERENCE TO SEQUENCE LISTING

This application is being filed electronically via EFS-Web and includes an electronically submitted sequence listing in .txt format. The .txt file contains a sequence listing entitled “PC33815A_SequenceListing.txt” created on Jul. 29, 2010 and having a size of 27.8 KB. The sequence listing contained in this .txt file is part of the specification and is herein incorporated by reference in its entirety.

### FIELD OF THE INVENTION

The present disclosure relates to immunogens, immunogenic compositions, and pharmaceutical compositions comprising an antigenic tau peptide that is linked to an immunogenic carrier, such as a virus-like particle (VLP), for the treatment of tau-related neurological disorders or conditions, such as Alzheimer's disease and Mild Cognitive Impairment. The disclosure further relates to methods of producing these immunogens, immunogenic compositions and pharmaceutical compositions and their use in medicine.

### BACKGROUND

Alzheimer's disease also referred to as Alzheimer's dementia or AD is a progressive neurodegenerative disorder or condition that causes memory loss and serious mental deterioration. AD is the most common form of dementia, accounting for more than half of all dementias. It is estimated that over 26 million people worldwide suffer from the effects of AD, a number that is expected to quadruple by 2050 as the population ages (Brookmeyer et al., Alzheimer's & Dementia 3:186-191 (2007)). In addition to the loss of life and reduced quality of life, the economic cost to society is enormous given that the average AD patient lives 8 to 10 years following diagnosis and requires high levels of daily care. Early on, patients complaining of slight memory loss and confusion are characterised as suffering from Mild Cognitive Impairment (MCI), which in some instances advances to the classical symptoms of Alzheimer's disease resulting in severe impairment of intellectual and social abilities.

Alzheimer's disease (AD) is typically characterised by the accumulation of neuritic plaques and neurofibrillary tangles in the brain, which result in the death of neuronal cells followed by progressive cognitive decline. Most of the currently available therapies for AD focus on treating the symptoms, but do not necessarily stop the progression of the disease. Accordingly, it is clear that new approaches are desirable to identify therapies that can protect neurons from the debilitating effects of AD.

Most current therapeutic approaches for treating AD are based on the broadly accepted “amyloid cascade hypothesis.” This concept ascribes a pathophysiological role to amyloid-β (Aβ) as a neuro- and synaptotoxin in monomer to oligomer form, as well as being deposited as polymer in amyloid plaques, one of the characteristic features of AD pathology. Monoclonal antibodies against the range of Aβ forms are believed to be efficacious because they shift the brain-blood equilibrium towards the blood, thereby depleting brain Aβ stores.

The pathophysiology of AD is characterised by more than just the deposition of Aβ into senile plaques, and also includes the accumulation of neurofibrillary tangles (NFTs). NFTs are fibrils formed by paired helical filaments that are linked together with hyperphosphorylated tau protein. Tau can be transiently phosphorylated by various kinases at more than 30 different serine and threonine residues (Hanger et al., J. Neurochem. 71:2465-2476 (1998)) as well as several tyrosine residues (Lebouvier et al, JAD 18: 1-9 (2009)). In AD, there is apparently an imbalance of kinase and phosphatase activities, resulting in hyperphosphorylated forms of tau protein that aggregate and accumulate as NFTs.

Mild Cognitive Impairment (MCI) is most commonly defined as having measurable memory impairment beyond that normally expected for aging, but not yet showing other symptoms of dementia or AD. MCI appears to represent a transitional state between cognitive changes associated with normal aging and early dementias. When memory loss is the predominant symptom, this type of MCI is further subdefined as amnestic MCI. Individuals with this subtype of MCI are most likely to progress to AD at a rate of approximately 10-15% per year (Grundman M et al, Arch Neurol. 61, 59-66, 2004). A large study published in 2005 was the first clinical trial to demonstrate that treatment of MCI patients could delay transition to AD during the first year of the trial (Petersen R C et al, NEJM 352, 2379-2388, 2005), indicating that these patients also represent a viable population for treatment intervention for AD.

A recent study reported that vaccination against phosphorylated tau peptides in a tangle mouse model of pathological tau resulted in a reduction in aggregated tau in the brain and improvements in the tangle-related behavioral deficits (Asuni et al., J. Neurosci. 27:9115-9129 (2007)). While the effect of hyperphosphorylated tau and NFTs on the loss of cognition and progression of AD is not completely understood, recent opinions suggest that targeting amyloid alone will not be sufficient to see improvement over the course of the disease, making additional or alternative targeting necessary (Oddo et al., J. Biol. Chem. 281:39413 (2006)). With this in mind, an active vaccine approach that targets the disease conformations of the tau protein may be necessary to generate an effective therapeutic vaccine for AD and MCI.

Furthermore, there are a number of diseases beyond AD and MCI which are also associated with tau pathology or “tauopathies” which could potentially benefit from a tau vaccine specifically targeting the involved pathological forms. These diseases include Frontotemporal dementia, Parkinson's disease, Pick's disease, Progressive supranuclear palsy, and Amyotrophic lateral sclerosis/parkinsonism-dementia complex to name a few (see, e.g. Spires-Jones et al, TINS 32:150-9 (2009)).

### SUMMARY

The present disclosure provides novel immunogens, immunogenic compositions and pharmaceutical compositions that comprise at least one antigenic tau peptide that is capable of inducing an immune response, in particular antibody responses, leading to antibody titer against the self-antigen tau in its pathological hyper-phosphorylated state. Such immunogens, immunogenic compositions and pharmaceutical compositions exhibit numerous desirable properties, such as the ability to induce an immune response, in particular antibody responses, with therapeutic effect against the induction and development of neurodegenerative diseases associated with hyper-phosphorylated tau, such as Alzheimer's disease and MCI.

In one aspect, the disclosure provides an immunogen comprising at least one antigenic tau peptide linked to an immunogenic carrier, wherein said antigenic tau peptide comprises a phospho-tau epitope selected from a pSer-396 phospho-tau epitope, a pThr-231/pSer-235 phospho-tau epitope, a pThr-231 phospho-tau epitope, a pSer-235 phospho-tau epitope, a pThr-212/pSer-214 phospho-tau epitope, a pSer-202/pThr-205 phospho-tau epitope., and epitope.

In one example, said phospho-tau epitope is a pSer-396 phospho-tau epitope. In a further example, said phospho-tau epitope is a pThr-231/pSer-235 phospho-tau epitope. In a further example, said phospho-tau epitope is a pThr-231 phospho-tau epitope, In a further example, said phospho-tau epitope is a pSer-235 phospho-tau epitope. In a further example, said phospho-tau epitope is a pThr-212/pSer-214 phospho-tau epitope. In a further example, said phospho-tau epitope is a pSer-202/pThr-205 phospho-tau epitope. In a further example, said phospho-tau epitope is a pTyr 18 phospho-tau epitope.

In another aspect, the disclosure provides an immunogen comprising at least one antigenic tau peptide linked to an immunogenic carrier, wherein said antigenic tau peptide comprises an amino acid sequence selected from SEQ ID NOs: 4, 6-26, 105 and 108-112.

In one example, said antigenic tau peptide is covalently linked to said immunogenic carrier by a linker represented by the formula (G)ₙC, where said linker is at either the C-terminus (peptide-(G)ₙC) or N-terminus (C(G)ₙ-peptide) of said peptide, and where n is 0, 1, 2, 3, 4, 5, 6, 7, 8, 9, or 10. In a further example, said linker is at the N-terminus of said tau peptide, and where n is 1 or 2. In another example, said linker is at the C-terminus of said tau peptide, and where n is 1 or 2. In a further example, said antigenic tau peptide comprises an amino acid sequence selected from SEQ ID NOs: 4 and 6-13. In a further example, said antigenic tau peptide consists of an amino acid sequence selected from SEQ ID NOs: 4 and 6-13. In a further example, said antigenic tau peptide consists of the amino acid sequence set forth in SEQ ID NO:11.

In another example, said antigenic tau peptide comprises an amino acid sequence selected from SEQ ID NOs:14-19. In a further example, said antigenic tau peptide consists of an amino acid sequence selected from SEQ ID NOs:14-19. In a further example, said antigenic tau peptide consists of the amino acid sequence set forth in SEQ ID NO:16.

In another example, said antigenic tau peptide comprises an amino acid sequence selected from SEQ ID NOs:20-24. In a further example, said antigenic tau peptide consists of an amino acid sequence selected from SEQ ID NOs:20-24. In a further example, said antigenic tau peptide consists of the amino acid sequence set forth in SEQ ID NO:21.

In another example, said antigenic tau peptide comprises an amino acid sequence selected from SEQ ID NOs: 105 and 108-112. In a further example, said antigenic tau peptide consists of an amino acid sequence selected from SEQ ID NOs: 105 and 108-112. In a further example, said antigenic tau peptide consists of the amino acid sequence set forth in SEQ ID NO:105.

In one aspect, the present disclosure provides any of the immunogens described herein, wherein said immunogenic carrier is a hemocyanin (such as KLH), a serum albumin, a globulin, a protein extracted from ascaris, or an inactivated baterial toxin.

In one aspect the present disclosure provides any of the immunogens described herein, wherein said immunogenic carrier is a virus-like particle selected from the group consisting of HBcAg VLP, HBsAg VLP, and Qbeta VLP. In one example, the disclosure provides a composition comprising at least two immunogens as described herein. In a further example, the composition comprises at least three immunogens as described herein.

In one example, the present disclosure provides a composition comprising at least two immunogens as described herein, wherein:

a) the antigenic tau peptide of the first immunogen consists of an amino acid sequence selected from SEQ ID NOs: 4 and 6-13; and

b) the antigenic tau peptide of the second immunogen consists of an amino acid sequence selected from SEQ ID NOs: 14-19.

In another example, the present disclosure provides a composition comprising at least two immunogens as described herein, wherein:

a) the antigenic tau peptide of the first immunogen consists of an amino acid sequence selected from SEQ ID NOs: 4 and 6-13; and

b) the antigenic tau peptide of the second immunogen consists of an amino acid sequence selected from SEQ ID NOs: 20-24.

In another example, the disclosure provides a composition comprising at least two immunogens as described herein, wherein:

a) the antigenic tau peptide of the first immunogen consists of an amino acid sequence selected from SEQ ID NOs: 14-19; and

b) the antigenic tau peptide of the second immunogen consists of an amino acid sequence selected from SEQ ID NOs: 20-24.

In a further example, the present disclosure provides a composition comprising at least two immunogens as described herein, wherein:

a) the antigenic tau peptide of the first immunogen consists of an amino acid sequence selected from SEQ ID NOs: 4 and 6-13; and

b) the antigenic tau peptide of the second immunogen selected from SEQ ID NO: 105 and 108-112.

In a further example, the present disclosure provides a composition comprising at least two immunogens as described herein, wherein:

a) the antigenic tau peptide of the first immunogen consists of an amino acid sequence selected from SEQ ID NOs: 14-19; and

b) the antigenic tau peptide of the second immunogen selected from SEQ ID NO: 105 and 108-112.

In a further example, the present disclosure provides a composition comprising at least two immunogens as described herein, wherein:

a) the antigenic tau peptide of the first immunogen consists of an amino acid sequence selected from SEQ ID NOs: 20-24; and

b) the antigenic tau peptide of the second immunogen selected from SEQ ID NO: 105 and 108-112.

In another example, the disclosure provides a composition comprising at least three of four immunogens as described herein, wherein:

a) the antigenic tau peptide of the first immunogen consists of an amino acid sequence selected from SEQ ID NOs:4, and 6-13;

b) the antigenic tau peptide of the second immunogen consists of an amino acid sequence selected from SEQ ID NOs:14-19; and

c)) the antigenic tau peptide of the third immunogen consists of an amino acid sequence selected from SEQ ID NOs:20-24.

d) the antigenic tau peptide of the fourth immunogen selected from SEQ ID NO: 105 and 108-112.

In a further example, the disclosure provides any of the compositions described herein, wherein each of said antigenic tau peptides is independently covalently linked to said immunogenic carrier by a linker represented by the formula (G)ₙC, where each of said linkers is independently at either the C-terminus (peptide-(G)ₙC) or N-terminus (C(G)ₙ-peptide) of said tau peptide, and where each n is independently 0, 1, 2, 3, 4, 5, 6, 7, 8, 9, or 10. In a further example, the disclosure provides any of the compositions described herein, wherein each of said linkers is at the N-terminus of the tau peptide and where each n is independently 1 or 2.

In another aspect, the present disclosure provides a composition comprising at least three of four immunogens, wherein:

a) the first immunogen comprises at least one antigenic tau peptide linked to a Qbeta VLP, wherein said antigenic tau peptide consists of SEQ ID NO:11, and where said peptide is covalently linked to said VLP by a linker represented by the formula (G)ₙC, where said linker is at either the C-terminus (peptide-(G)ₙC) or N-terminus (C(G)ₙ-peptide) of said tau peptide, and where n is 1, or 2;

b) the second immunogen comprises at least one antigenic tau peptide linked to a Qbeta VLP, wherein said antigenic tau peptide consists of SEQ ID NO:16, and where said peptide is covalently linked to said VLP by a linker represented by the formula (G)ₙC, where said linker is at either the C-terminus (peptide-(G)ₙC) or N-terminus (C(G)ₙ-peptide) of said tau peptide, and where n is 1, or 2; and

c) the third immunogen comprises at least one antigenic tau peptide linked to a Qbeta VLP, wherein said antigenic tau peptide consists of SEQ ID NO:21, and where said peptide is covalently linked to said VLP by a linker represented by the formula (G)ₙC, where said linker is at either the C-terminus (peptide-(G)ₙC) or N-terminus (C(G)ₙ-peptide) of said tau peptide, and where n is 1, or 2.

d)) the fourth immunogen comprises at least one antigenic tau peptide linked to a Qbeta VLP, wherein said antigenic tau peptide consists of SEQ ID NO:105, and where said peptide is covalently linked to said VLP by a linker represented by the formula (G)ₙC, where said linker is at either the C-terminus (peptide-(G)ₙC) or N-terminus (C(G)ₙ-peptide) of said tau peptide, and where n is 1, or 2.

In one example, each of the linkers of the first, second and third immunogens are at the N-terminus of each of the antigenic tau peptides and wherein for each of said linkers, n is 2.

In another aspect, the present disclosure provides a composition comprising any of the immunogens or compositions described herein, further comprising at least one adjuvant selected from alum, CpG-containing oligonucleotides, and saponin-based adjuvants.

In a further aspect, the present disclosure provides a pharmaceutical composition comprising any of the immunogens or compositions described herein, and a pharmaceutically acceptable excipient. In one example, at least one adjuvant is a CpG-containing oligonucleotide selected from CpG 7909 (SEQ ID NO: 27), CpG 10103 (SEQ ID NO:28), and CpG 24555 (SEQ ID NO: 29).

In a further aspect, the present disclosure provides a pharmaceutical composition comprising any of the immunogens or compositions described herein, and a pharmaceutically acceptable excipient.

In another aspect, the present disclosure provides a method of immunization comprising administering to a mammal any of the immunogens, compositions, or pharmaceutical compositions described herein. For example, in one aspect, such administration occurs by using a pharmaceutically effective dose of any of the immunogens, compositions, or pharmaceutical compositions described herein.

In another aspect, the disclosure provides a method of treating a tau-related neurological disorder in a mammal comprising administering to said mammal a therapeutically effective amount of any of the immunogens, immunogenic compositions, or pharmaceutical compositions described herein.

In one aspect, such administration occurs by using a pharmaceutically effective dose of any of the immunogens, compositions, or pharmaceutical compositions described herein.

In another aspect, the disclosure provides a method of treating a tau-related neurological disorder in a mammal comprising administering to said mammal: a) a pharmaceutically effective dose of any of the immunogens, immunogenic compositions, or pharmaceutical compositions described herein; and b) a pharmaceutically effective dose of at least one adjuvant. In one example, the at least one adjuvant is selected from alum, CpG-containing oligonucleotides, and saponin-based adjuvants. In a further example, the at least one adjuvant is a CpG-containing oligonucleotide selected from CpG 7909 (SEQ ID NO: 27), CpG 10103 (SEQ ID NO:28), and CpG 24555 (SEQ ID NO: 29).

In a further example, said neurological disorder is Alzheimer's disease. In another example, said neurological disorder is diagnosed as Mild Cognitive Impairment. In another example, said neurological disorder is diagnosed as Amnestic MCI.

In another example, the disclosure provides a use of any of the immunogens, compositions, or pharmaceutical compositions described herein for the manufacture of a medicament. For example, in one aspect, such medicaments can be used for the treatment of a tau-related neurological disorder in a mammal. In one example, said neurological disorder is Alzheimer's disease. In another example, said neurological disorder is diagnosed as Mild Cognitive Impairment (MCI). In another example, said neurological disorder is diagnosed as Amnestic MCI.

In a further aspect, the disclosure provides an isolated antibody that is produced in response to any of the immunization methods described herein, wherein said antibody specifically binds to a hyperphosphorylated form of human tau.

In a further aspect, the disclosure provides a method of treating a tau-related neurological disorder in a mammal comprising administering to said mammal an antibody that specifically binds to a hyperphosphorylated form of human tau and wherein said antibody is produced in response to any of the immunization methods described herein.

In a further aspect, the disclosure provides a use of any of the antibodies described herein for the manufacture of a medicament for the treatment of a tau-related neurological disorder in a mammal. In one example, said neurological disorder is Alzheimer's disease. In another example, said neurological disorder is diagnosed as Mild Cognitive Impairment (MCI). In another example, said neurological disorder is diagnosed as Amnestic MCI.

In a further aspect, the present disclosure provides an isolated peptide consisting, or consisting essentially of, of an amino acid sequence selected from SEQ ID NOs: 4, 6 to 26, 31 to 76 and 105 to 122. In a further aspect, the present disclosure provides an isolated nucleic acid that encodes any of said isolated peptides. In a further aspect, the present disclosure provides an expression vector comprising any of said nucleic acids. In a further aspect the present disclosure provides a host cell comprising any of said expression vectors.

### BRIEF DESCRIPTION OF THE DRAWINGS

FIGS. 1A and 1B shows a description of the groups of Balb/c mice that were immunized subcutaneously, and the titer and selectivity results, as described in Example 5. Balb/c mice were immunized subcutaneously with 300 μg of peptide, 100 μg of peptide-KLH or 100 μg of peptide-VLP. 50 μL of TiterMax Gold (Alexis Biochemicals) was used as adjuvants where listed. Serum dilutions tested in the antigen specific titier determination assay (see Example 13) ranged from 1:30 to 1:7,290.

FIG. 2 shows a description of the groups of Balb/c mice that were immunized, and the titer results as described in Example 5. Balb/c mice were immunized subcutaneously. 50 μL of TiterMax Gold was used as an adjuvant where listed. Serum dilutions tested in the antigen specific titier determination assay (see Example 13) ranged from 1:900 to 1:1,968,300.

FIG. 3 shows a description of Balb/c mice that were immunized subcutaneously as further described in Example 6. 100 μg of peptide was used for prime and 100 μg of peptide-VLP was used for the boosts. 750 μg of alum (Al(OH)₃) was used as adjuvants where listed. Serum dilutions tested in the antigen specific titier determination assay (see Example 13) ranged from 1:800 to 1:1,750,000. ND means not determined.

FIGS. 4A, 4B, and 4C show the results of TG4510++ mice that were immunized intramuscularly, as described in Example 7. FIG. 4A shows the titer results for Groups 1 to 7, while FIG. 4B shows the titer results for Groups 8 to 17. FIG. 4C shows the selectivity results for Groups 1 to 6. CPG is CpG-24555. Alum is Al(OH)₃. Serum dilutions tested in the antigen specific titier determination assay (see Example 13) ranged from 1:5,000 to 1:15,800,000. ND means not determined.

FIG. 5 shows a description of mice that were immunized as described in Example 8. Balb/c mice were immunized via either intramuscular (IM) or subcutaneous (SC) route. 90 μg of peptide-VLP was used where listed. 1,595 μg of Alum (Al(OH)₃), 20 μg CpG-24555 and 12 μg ABISCO-100 were used where listed. Serum dilutions tested in the antigen specific titier determination assay (see Example 13) ranged from 1:5,000 to 1:15,800,000. The lower limit of detection of the standard curve was 0.0025 mg/mL. NA means not applicable.

FIG. 6 shows a description of mice that were immunized as described in Example 11. Balb/c mice were immunized intramuscularly. 100 μg of peptide-VLP was used. 252 (750) μg of Alum (Al(OH)₃) was used where listed. Serum dilutions tested in the antigen specific titier determination assay (see Example 13) ranged from 1:500 to 1:2,720,000. ND means not determined.

FIG. 7 shows a description of mice that were immunized as described in Example 11. Balb/c mice were immunized intramuscularly. 750 μg of Alum (Al(OH)₃) was used as an adjuvant. Serum dilutions tested in the antigen specific titier determination assay (see Example 13) ranged from 1:500 to 1:15,800,000.

FIG. 8 shows a description of mice that were immunized as described in Example 12. TG4510−/− (wild type littermate) mice were immunized intramuscularly. 100 μg of each peptide-VLP was used for day 0 prime and day 14 boost, as listed. The listed amount of alum (Al(OH)₃) was used. The sera from the ‘No Treatment’ group were pooled. Serum dilutions tested in the antigen specific titier determination assay (see Example 13) ranged from 1:5,000 to 1:15,800,000.

FIG. 9 shows a description of mice that were immunized as described in Example 12. TG4510−/− (wild type littermate) mice were immunized intramuscularly. 100 μg of each peptide-VLP was used for day 0 prime and day 14 boost. No alum or 504 μg of alum (Al(OH)₃) was used. Spleens were collected on day 21. The numbers of spots per 5×10⁵ spleen cells is shown as measured by Interferon-gamma T-cell ELIspot (see Example 14). Results are from a pool of 3 spleens. Peptide HBV-1 (SEQ ID NO:77) was the irrelevant peptide. BSA was the irrelevant protein. ND indicates not determined. * indicates p<0.05 versus the irrelevant peptide or protein as appropriate.

FIG. 10 shows the amino acid sequence of human tau isoform 2, Genbank Accession No. NP_{—}005901 (SEQ ID NO:30).

### DETAILED DESCRIPTION

### Definitions and General Techniques

Unless otherwise defined herein, scientific and technical terms used in connection with the present disclosure shall have the meanings that are commonly understood by those of ordinary skill in the art. Generally, nomenclature used in connection with, and techniques of, cell and tissue culture, molecular biology, immunology, microbiology, genetics and protein and nucleic acid chemistry, hybridization, analytical chemistry, synthetic organic chemistry, and medicinal and pharmaceutical chemistry described herein are those well known and commonly used in the art.

The methods and techniques of the present disclosure are generally performed according to conventional methods well known in the art and as described in various general and more specific references that are cited and discussed throughout the present specification unless otherwise indicated. See, e.g., Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). Enzymatic reactions and purification techniques are performed according to manufacturer's specifications, as commonly accomplished in the art or as described herein.

The term “mild cognitive impairment (MCI),” as used herein, refers to a category of memory and cognitive impairment that is typically characterised by a clinical dementia rating (CDR) of 0.5 (see, e.g., Hughes et al., Brit. J. Psychiat. 140: 566-572,1982) and further characterised by memory impairment, but not impaired function in other cognitive domains. Memory impairment is preferably measured using tests such as a “paragraph test.” A patient diagnosed with Mild Cognitive Impairment often exhibits impaired delayed recall performance. Mild Cognitive Impairment is typically associated with ageing and generally occurs in patients who are 45 years of age or older.

The term “dementia,” as used herein, refers to a psychiatric condition in its broadest sense, as defined in American Psychiatric Association: Diagnostic and Statistical Manual of Mental Disorders, Fourth Edition, Washington, D.C., 1994 (“DSM-IV”). The DSM-IV defines “dementia” as characterised by multiple cognitive deficits that include impairments in memory and lists various dementia according to presumed etiology. The DSM-IV sets forth a generally accepted standard for such diagnosing, categorizing and treating of dementia and associated psychiatric disorders.

The terms “Tau” or “tau protein” refers to the tau protein which is associated with the stabilization of microtubules in nerve cells and a component of a broad range of tau aggregates, e.g., neurofibrillary tangles. In particular, the term “tau protein” as used herein encompasses any polypeptide comprising, or consisting of, the human tau of SEQ ID NO: 30, or other human isoforms with or without modifications, or the corresponding orthologs from any other animals. The term “tau protein” as used herein further encompasses post-translational modifications including but not limited to glycosylations, acetylations, and phosphorylations of the tau protein as defined above.

The term “Tauopathy” refers to tau-related disorders or conditions, e.g., Alzheimer's Disease, Progressive Supranuclear Palsy (PSP), Corticobasal Degeneration (CBD), Pick's Disease, Frontotemporal dementia and Parkinsonism associated with chromosome 17 (FTDP-17), Parkinson's disease, stroke, traumatic brain injury, mild cognitive impairment and the like.

The terms “antigen,” and “immunogen”, which are meant to be interchangeable as used herein, refer to a molecule capable of being bound by an antibody, a B cell receptor (BCR), or a T cell receptor (TCR) if presented by MHC molecules. The terms “antigen” and “immunogen”, as used herein, also encompass T-cell epitopes. An antigen can additionally be capable of being recognized by the immune system and/or being capable of inducing a humoral immune response and/or cellular immune response leading to the activation of B- and/or T-lymphocytes. This may, however, require that, at least in certain cases, the antigen contains or is linked to a T helper cell epitope and is given an adjuvant. An antigen can have one or more epitopes (e.g., B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. The terms “antigen” and “immunogen” both encompass, but are not limited to, polypeptides.

The term “antigenic site” and the term “antigenic epitope”, which are used herein interchangeably, refer to continuous or discontinuous portions of a polypeptide, which can be bound immunospecifically by an antibody or by a T-cell receptor within the context of an MHC molecule. Immunospecific binding excludes non-specific binding but does not necessarily exclude cross-reactivity. Antigenic sites typically comprise 5 to 10 amino acids in a spatial conformation which is unique to the antigenic site.

As used herein, the term “phosphorylated” in reference to an amino acid residue refers to the presence of a phosphate group on the side chain of the residue where a hydroxyl group is otherwise normally present. Such phosphorylation typically occurs as a substitution of the hydrogen atom from a hydroxyl group for a phosphate group (—PO₃H₂). As recognized by those of skill in the art, depending on the pH of the local environment, this phosphate group can exist as an uncharged, neutral group (—PO₃H₂), or with a single (—PO₃H⁻), or double (—PO₃²⁻) negative charge. Amino acid residues that can typically be phosphorylated include the side chains of serine, threonine, and tyrosine. Throughout the present disclosure an amino acid residue that is phosphorylated is indicated by bold text and underlined.

As used herein, reference to amino acid residues are denoted by the one-letter or three-letter code (see, e.g. Lehninger, Biochemistry, 2^{nd} edition, Worth Publishers, New York, 1975, p. 72).

The articles “a” and “an” are used herein to refer to one or to more than one (i.e., to at least one) of the grammatical object of the article. By way of example, “an element” means one element or more than one element. Further, unless otherwise required by context, singular terms shall include pluralities and plural terms shall include the singular unless the content clearly dictates otherwise.

The term “peptide” or “polypeptide” refers to a polymer of amino acids without regard to the length of the polymer; thus, protein fragments, oligopeptides, and proteins are included within the definition of peptide or polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “tau fragment” as used herein encompasses any polypeptide comprising, or consisting of, at least 3, 4, 5, 6, 7, 8, 9, 10, 11, 12, 13, 14, 15, 16, 17, 18, 19, 20, 21, 22, 23, 24, 25, 26, 27, 28, 29, or 30 contiguous amino acids of a tau protein as defined herein.

The term “pSer-396 phospho-tau epitope” as used herein refers to a peptide comprising the amino acid sequence KSP (i.e. Lys-395 Ser-396 Pro-397 from the human tau sequence), where the serine residue is phosphorylated, and wherein the sequence numbering is based on the human tau isoform 2 that is provided as SEQ ID NO:30. A pSer-396 phospho-tau epitope is typically about 3 to about 25 amino acids in length.

The term “pThr-231/pSer-235 phospho-tau epitope” as used herein refers to a peptide comprising the amino acid sequence TPPKS (SEQ ID NO:1) (i.e. Thr-231 Pro-232 Pro-233 Lys-234 Ser-235 from the human tau sequence), where the threonine and serine residues are each phosphorylated, and wherein the sequence numbering is based on the human tau isoform 2 that is provided as SEQ ID NO:30. Such epitopes are typically about 5 to about 25 amino acids in length. The pThr-231/pSer-235 phospho-tau epitope can also refer to a form of this epitope that comprises the phosphorylated Thr-231 residue, but does not include the phosphorylated Ser-235 residue, or comprises the phosphorylated Ser-235 residue, but does not include the phosphorylated Thr-231 epitope. Such versions of this epitope are typically about 3 to about 20 amino acids in length.

The term “pThr-212/pSer-214 phospho-tau epitope” as used herein refers to a peptide comprising the amino acid sequence TPS (i.e. Thr-212 Pro-213 Ser-214 from the human tau sequence) where the threonine and serine residues are each phosphorylated, and wherein the sequence numbering is based on the human tau isoform 2 that is provided as SEQ ID NO:30. A pThr-212/pSer-214 phospho-tau epitope is typically about 3 to about 25 amino acids in length.

The term “pSer-202/pThr-205 phospho-tau epitope” as used herein refers to a peptide comprising the amino acid sequence SPGT (SEQ ID NO:3) (i.e. Ser-202 Pro-203 Gly-204 Thr-205 from the human tau sequence), where the serine and threonine residues are each phosphorylated, and wherein the sequence numbering is based on the human tau isoform 2 that is provided as SEQ ID NO:30. A pSer-202/pThr-205 phospho-tau epitope is typically about 4 to about 25 amino acids in length.

The terms “purified” and “isolated” as used herein are synonymous. For example, the terms “isolated” or “purified” with respect to a polypeptide refer to a polypeptide that by virtue of its origin or source of derivation (1) is not associated with naturally associated components that accompany it in its native state, (2) is substantially free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A polypeptide may also be rendered substantially free of naturally associated components by isolation, using protein purification techniques well known in the art. A polypeptide is “substantially pure,” “substantially homogeneous,” or “substantially purified” when at least about 60 to 75% of a sample exhibits a single species of polypeptide. The polypeptide may be monomeric or multimeric. A substantially pure polypeptide can typically comprise about 50%, 60%, 70%, 80% or 90% w/w of a polypeptide sample, more usually about 95%, and preferably can be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term tau-related neurological disorder, as used herein, means any disease or other condition in which tau (particularly hyperphosphorylated forms of tau) is believed to play a role. Such disorders, diseases, and/or conditions typically correlate with the presence of neurofibrillary tangles (typically involving hyperphosphorylated forms of tau), and include, without limitation, Alzheimer's disease, MCI, fronto-temporal dementia, Pick's disease, progressive nuclear palsy, corticobasal degeneration, parkinsonism-dementia complex of Guam, and other tauopathies.

The term “antigenic tau peptide”, as used herein, encompasses all tau-derived polypeptides, such as from mammalian species, for example from human, as well as their variants, analogs, orthologs, homologs and derivatives, and fragments thereof that exhibit an “antigenic tau peptide biological activity”. For example, the term “antigenic tau peptide” refers to polypeptides comprising, consisting of, or consisting essentially of, an amino acid sequence selected from SEQ ID NOs: 1 to 26, 31 to 76, and 105-122 as well as to their variants, homologs and derivatives exhibiting essentially the same biological activity.

The term “antigenic tau peptide biological activity”, as used herein, refers to the ability of the antigenic tau peptides of the disclosure to induce auto tau antibodies in a subject with an antagonistic profile, such auto-antibodies being able to decrease the level of hyperphosphorylated, pathological forms of tau, while being substantially unable to bind to normal non-hyperphosphorylated, non-pathological forms of tau. Furthermore, an antigenic tau peptide that has antigenic tau peptide biological activity can be designed to minimize a tau-specific T-cell response when administered to a patient. It will be apparent to those skilled in the art which techniques may be used to confirm whether a specific construct falls within the scope of the present disclosure or not. Such techniques include, but are not restricted to the techniques described in the Examples section of the present disclosure, and also to the following. A peptide with putative antigenic tau peptide biological activity can be assayed to ascertain the immunogenicity of the peptide (e.g. to determine whether antisera raised by the putative peptide will bind hyperphosphorylated forms of tau, but does not substantially bind non-hyperphosphorylated, non-pathological forms of tau). Further, a peptide with putative antigenic tau peptide biological activity can be assayed to determine whether or not the peptide substantially induces a tau-specific T-cell mediated response.

The term “hyperphosphorylated” or “abnormally phosphorylated” as used herein, refers to tau that contains at least about 7 (i.e. about 7 or more) phosphate groups per tau molecule (see, e.g. Kopke et al., J. Biol Chem 268:24374-84 (1993)). Hyperphosphorylated tau is a major component of neurofibrillary tangles (NFTs) and paired helical filaments (PHFs) found in AD patients, and hyperphosphorylation is responsible for tau's loss of normal biological activity and self-aggregation. Some tau residues are typically only found phosphorylated in its pathological hyperphosphorylated forms such as PHFs and NFTs. Such residues include Ser-202, Thr-205, Thr-212, Ser-214, Thr-231, Ser-235, Ser-396 and/or Ser-404, Tyr-18. Therefore, tau proteins phosphorylated at multiple sites not normally involved in tau binding to microtubules, in particular at those sites found in the proline rich regions flanking the microtubule binding region of tau and comprising a major component of PHFs and NFTs, are also included in the term hyperphosphorylated tau, or abnormally phosphorylated tau.

### Antigenic Tau Peptides

Human tau protein is a microtubule-associated protein that is relatively abundant in neurons of the central nervous system, but is less common in other locations. In brain tissue, tau exists as six different isoforms as a result of alternative splicing in exons 2, 3, and 10 of the tau gene. Human tau isoform 2 (SEQ ID NO:30) is used herein as the reference for the amino acid numbering with regard to all tau peptides of the present disclosure. Tau normally interacts with tubulin to stabilize microtubules and promote tubulin assembly into microtubules, as well as providing axonal transport of proteins. Tau is a developmentally regulated phosphoprotein, typically containing 2 to 3 phosphate groups per molecule in its normal state in human adult brains. However, tau can be transiently phosphorylated by different kinases at more than 30 different residues, mostly at the Ser/Thr-Pro motif (Hanger et al., J. Neurochem. 71:2465-2476 (1998)).

Antigenic tau peptides of the present disclosure will typically be of a small size, such that they mimic a region selected from the whole tau protein in which an epitope in a pathological form of tau is found. As described previously, such pathological forms of tau are typically characterised by phosphorylation at certain amino acids within the tau protein. The antigenic tau peptides of the disclosure, therefore, are typically less than 100 amino acids in length, for example less than 75 amino acids, for example less than 50 amino acids. The antigenic tau peptides of the disclosure are typically about 3, 4, 5, 6, 7, 8, 9, 10, 11, 12, 13, 14, 15, 16, 17, 18, 19, 20, 21, 22, 23, 24, 25, 26, 27, 28, 29 or about 30 amino acids in length. Specific examples of antigenic tau peptides of the disclosure provided in the sequence listing include peptides ranging from 4 to 31 amino acids in length. As will be apparent to those skilled in the art, such antigenic peptides typically have a free N-terminus, and can have either a carboxylated or amidated C-terminus.

The antigenic peptides of the disclosure comprise an amino acid sequence derived from a portion of human tau in its hyperphosphorylated, or pathological form. In particular, such antigenic tau peptides will typically comprise the specific phospho-tau epitopes which can be referred to in the literature with reference to antibodies that bind these epitopes (such as PHF1, TG3, ATB, and/or AT100; see, e.g. Hanger et al., J. Biol. Chem. 282 (32):23645-23654 (2007); Pennanen et al., Biochem. Biophys. Res. Comm. 337:1097-1101 (2005); Porzig et al., Biochem. Biophys. Res. Comm. 358:644-649 (2007)).

The present disclosure has identified specific antigenic regions of the human tau protein that when used alone, or in combination with each other, can be beneficially used to elicit an immune response against pathological forms of hyperphosphorylated tau. For example, the pSer-396 phospho-tau epitope is typically a fragment of human tau that includes the phosphorylated serine residue Ser-396. Such fragments are typically about 3 to about 20 amino acids in length (e.g. 3, 4, 5, 6, 7, 8, 9, 10, 11, 12, 13, 14, 15, 16, 17, 18, 19, or 20), and include at least one amino acid from the native human tau sequence on both the N-terminal and C-terminal sides of Ser-396. For example, a pSer-396 phospho-tau epitope will typically comprise residues 395, 396, and 397 of the human tau sequence as set forth in SEQ ID NO:30 (i.e. Lys-395 Ser-396 Pro-397, where Ser-396 is phosphorylated). Such pSer-396 epitopes can also further comprise the phosphorylated serine residue Ser-404 of the native human sequence. Examples of tau peptides comprising a pSer-396 phospho-tau epitope are provided as SEQ ID NOs:4, and 6-13.

Further, for example, the pThr-231/pSer-235 phospho-tau epitope is typically a fragment of human tau that includes both the phosphorylated threonine residue Thr-231 and the phosphorylated serine residue Ser-235. Alternatively, a pThr-231/pSer-235 phospho-tau epitope includes only one of Thr-231 or Ser-235. Such epitopes are typically about 3 to about 20 amino acids in length (e.g. 3, 4, 5, 6, 7, 8, 9, 10, 11, 12, 13, 14, 15, 16, 17, 18, 19, or 20) and include at least one amino acid from the native human tau sequence on the N-terminal side of Thr-231 (i.e. Arg-230) and/or at least one amino acid on the C-terminal side of Ser-235 (i.e. Pro-236). Examples of tau peptides comprising a pThr-231/pSer-235 epitope are provided as SEQ ID NOs: 14-19.

Further, for example, the pThr-212/pSer-214 phospho-tau epitope is typically a fragment of human tau that includes the phosphorylated threonine residue Thr-212 and the phosphorylated serine residue Ser-214. Such epitopes are typically about 3 to about 20 amino acids in length (e.g. 3, 4, 5, 6, 7, 8, 9, 10, 11, 12, 13, 14, 15, 16, 17, 18, 19, or 20) and include at least one amino acid from the native human tau sequence on the N-terminal side of Thr-212 (i.e. Arg-211) and at least one amino acid on the C-terminal side of Ser-214 (i.e. Leu-215). Examples of tau peptides comprising a pThr-212/pSer-214 epitope are provided as SEQ ID NOs: 20-24.

Further, for example, the pSer-202/pThr-205 phospho-tau epitope is typically a fragment of human tau that includes the phosphorylated serine residue Ser-202 and the phosphorylated threonine residue Thr-205. Such epitopes are typically about 6 to about 20 amino acids in length (e.g. 6, 7, 8, 9, 10, 11, 12, 13, 14, 15, 16, 17, 18, 19, or 20) and typically include at least one amino acid from the native human tau sequence on the N-terminal side of Ser-202 (i.e. Gly-201) and at least one amino acid on the C-terminal side of Thr-205 (i.e. Pro-206). An example of a tau peptide comprising an pSer-202/pThr-205 epitope is provided as SEQ ID NO: 25.

Further, for example, the pTyr-18 phospho-tau epitope is typically a fragment of human tau that includes the phosphorylated tyrosine residue Tyr-18. Such epitopes are typically about 6 to about 20 amino acids in length (e.g. 6, 7, 8, 9, 10, 11, 12, 13, 14, 15, 16, 17, 18, 19, or 20) and typically include at least one amino acid from the native human tau sequence on the N-terminal side of Tyr-18 (i.e. Thr-17) and at least one amino acid on the C-terminal side of Tyr-18 (i.e. Gly-19). An example of a tau peptide comprising a pTyr-18 epitope is provided as SEQ ID NO:112.

Antigenic tau peptides of the present disclosure can also include tau peptides comprising the phospho-tau epitopes described above, including peptides where a small number of amino acids have been substituted, added or deleted, but which retains essentially the same immunological properties. In addition, such derived antigenic tau peptides can be further modified by amino acids, especially at the N- and C-terminal ends to allow the antigenic tau peptide to be conformationally constrained and/or to allow coupling of the antigenic tau peptide to an immunogenic carrier after appropriate chemistry has been carried out.

The antigenic tau peptides of the disclosure also encompass functionally active variant peptides derived from the amino acid sequence of tau in which amino acids have been deleted, inserted or substituted without essentially detracting from the immunological properties thereof, i.e. such functionally variant peptides retain a substantial antigenic tau peptide biological activity. Typically, such functionally variant peptides have an amino acid sequence homologous, preferably highly homologous, to the amino acid sequences described in any of SEQ ID NOs: 1 to 26, 31 to 76, and 105-122

In one aspect, such functionally active variant peptides exhibit at least 60%, 65%, 70%, 75%, 80%, 85%, 90%, 95%, 96%, 97%, 98% or 99% identity to an amino acid sequence selected from the group consisting of SEQ ID NOs: 1 to 26, 31 to 76, and 105-122

The amino acid sequence identity of polypeptides can be determined conventionally using known computer programs such as Bestfit, FASTA, or BLAST (see, e.g. Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucelic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

Functionally active variants comprise naturally occurring functionally active variants such as allelic variants and species variants and non-naturally occurring functionally active variants that can be produced by, for example, mutagenesis techniques or by direct synthesis.

A functionally active variant differs by about, for example, 1, 2, 3, 4, 5, 6, 7, 8, 9, or 10 amino acid residues from any of the peptides set forth in SEQ ID NOs: 1 to 26 and 31 to 76, and yet retains an antigenic tau biological activity. Where this comparison requires alignment, the sequences are aligned for maximum homology. The site of variation can occur anywhere in the peptide, as long as the biological activity is substantially similar to any of the peptides set forth in SEQ ID NOs: 1 to 26, 31 to 76, and 105-122

Guidance concerning how to make phenotypically silent amino acid substitutions is provided in Bowie et al., Science, 247: 1306-1310 (1990), which teaches that there are two main strategies for studying the tolerance of an amino acid sequence to change.

The first strategy exploits the tolerance of amino acid substitutions by natural selection during the process of evolution. By comparing amino acid sequences in different species, the amino acid positions which have been conserved between species can be identified. These conserved amino acids are likely important for protein function. In contrast, the amino acid positions in which substitutions have been tolerated by natural selection indicate positions which are not critical for protein function. Thus, positions tolerating amino acid substitution can be modified while still maintaining specific immunogenic activity of the modified peptide.

The second strategy uses genetic engineering to introduce amino acid changes at specific positions of a cloned gene to identify regions critical for protein function. For example, site-directed mutagenesis or alanine-scanning mutagenesis can be used (Cunningham et al., Science, 244: 1081-1085 (1989)). The resulting variant peptides can then be tested for specific antigenic tau biological activity.

According to Bowie et al., these two strategies have revealed that proteins are surprisingly tolerant of amino acid substitutions. The authors further indicate which amino acid changes are likely to be permissive at certain amino acid positions in the protein. For example, the most buried or interior (within the tertiary structure of the protein) amino acid residues require nonpolar side chains, whereas few features of surface or exterior side chains are generally conserved.

Methods of introducing a mutation to amino acids of a protein is well known to those skilled in the art (see, e.g., Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989)).

Mutations can also be introduced using commercially available kits such as “QuikChange™ Site-Directed Mutagenesis Kit” (Stratagene). The generation of a functionally active variant to an antigenic tau peptide by replacing an amino acid which does not significantly influence the function of said antigenic tau peptide can be accomplished by one skilled in the art. One type of amino acid substitution that may be made in one of the peptides according to the present disclosure is a conservative amino acid substitution. A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art (see e.g. Pearson, Methods Mol. Biol. 243:307-31 (1994)).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

A functionally active variant peptide can also be isolated using a hybridization technique. Briefly, DNA having a high homology to the whole or part of a nucleic acid sequence encoding the peptide, polypeptide or protein of interest, e.g. SEQ ID NOs: 1 to 26, 31 to 76, and 105-122, is used to prepare a functionally active peptide. Therefore, an antigenic tau peptide of the disclosure also includes peptides that are functionally equivalent to any of SEQ ID NOs: 1 to 26 and 31 to 76 and can be encoded by a nucleic acid molecule that hybridizes with a nucleic acid encoding any of SEQ ID NOs: 1 to 26, 31 to 76, and 105-122, or a complement thereof. One of skill in the art can easily determine nucleic acid sequences that encode peptides disclosed herein using readily available codon tables. As such, these nucleic acid sequences are not presented herein.

The stringency of hybridization for a nucleic acid encoding a peptide, polypeptide or protein that is a functionally active variant is, for example, 10% formamide, 5×SSPE, 1×Denhart's solution, and 1× salmon sperm DNA (low stringency conditions). More preferable conditions are, 25% formamide, 5×SSPE, 1×Denhart's solution, and 1× salmon sperm DNA (moderate stringency conditions), and even more preferable conditions are, 50% formamide, 5×SSPE, 1×Denhart's solution, and 1× salmon sperm DNA (high stringency conditions). However, several factors influence the stringency of hybridization other than the above-described formamide concentration, and one skilled in the art can suitably select these factors to accomplish a similar stringency.

Nucleic acid molecules encoding a functionally active variant can also be isolated by a gene amplification method such as PCR using a portion of a nucleic acid molecule DNA encoding a peptide, polypeptide or protein of interest, e.g. any of the peptides set forth in SEQ ID NOs: 1 to 26, 31 to 76, and 105-122, as the probe.

### Production of Peptides/Proteins

Polypeptides of the present disclosure can be derived from natural sources and isolated from a mammal, such as, for example, a human, a primate, a cat, a dog, a horse, a mouse, or a rat. Polypeptides of the disclosure can thus be isolated from cells or tissue sources using standard protein purification techniques.

Alternatively, polypeptides can be synthesized chemically or produced using recombinant DNA techniques. For example, a polypeptide of the disclosure (e.g. a tau fragment) can be synthesized by solid phase procedures well known in the art. Suitable syntheses may be performed by utilising “T-boc” or “F-moc” procedures. Cyclic peptides can be synthesised by solid phase methods employing the well-known “F-moc” procedure and polyamide resin in a fully automated apparatus. Alternatively, those skilled in the art will know the necessary laboratory procedures to perform the process manually. Techniques and procedures for solid phase synthesis are described in Solid Phase Peptide Synthesis: A Practical Approach by E. Atherton and R. C. Sheppard, published by IRL at Oxford University Press (1989) and Methods in Molecular Biology, Vol. 35: Peptide Synthesis Protocols (ed. M. W. Pennington and B. M. Dunn), chapter 7, pp. 91-171 by D. Andreau et al.

Alternatively, a polynucleotide encoding a polypeptide of the disclosure can be introduced into an expression vector that can be expressed in a suitable expression system using techniques well known in the art, followed by isolation or purification of the expressed polypeptide of interest. A variety of bacterial, yeast, plant, mammalian, and insect expression systems are available in the art and any such expression system can be used. Optionally, a polynucleotide encoding a polypeptide of the disclosure can be translated in a cell-free translation system.

Antigenic tau peptides of the disclosure can also comprise those that arise as a result of the existence of multiple genes, alternative transcription events, alternative RNA splicing events, and alternative translational and posttranslational events. A polypeptide can be expressed in systems, e.g., cultured cells, which result in substantially the same posttranslational modifications present as when the polypeptide is expressed in a native cell, or in systems that result in the alteration or omission of posttranslational modifications, e.g., glycosylation or cleavage, present when expressed in a native cell.

A polypeptide of the disclosure, such as an antigenic tau polypeptide, can be produced as a fusion protein that contains other non-tau or non-tau-derived amino acid sequences, such as amino acid linkers or signal sequences or immunogenic carriers as defined herein, as well as ligands useful in protein purification, such as glutathione-S-transferase, histidine tag, and staphylococcal protein A. More than one antigenic tau polypeptide of the disclosure can be present in a fusion protein. The heterologous polypeptide can be fused, for example, to the N-terminus or C-terminus of the polypeptide of the disclosure. A polypeptide of the disclosure can also be produced as a fusion polypeptide comprising homologous amino acid sequences, i.e., other tau or tau-derived sequences.

### Constrained Peptides

The antigenic tau peptides of the disclosure may be linear or conformationally constrained. As used herein in reference to a molecule, the term “conformationally constrained” means a molecule, such as a polypeptide, in which the three-dimensional structure is maintained substantially in one spatial arrangement over time. Conformationally constrained molecules can have improved properties such as increased affinity, immunogenecity, metabolic stability, membrane permeability or solubility. In addition, such conformationally constrained molecules are expected to present the antigenic tau epitope in a conformation similar to its native conformation, thereby inducing anti-tau antibodies more susceptible to recognize self tau molecules. Methods of conformational constraint are well known in the art and include, without limitation, bridging and cyclization.

There are several approaches known in the prior art to introduce conformational constraints into a linear peptide or polypeptide chain. For example, bridging between two neighboring amino acids in a peptide leads to a local conformational modification, the flexibility of which is limited in comparison with that of regular peptides. Some possibilities for forming such bridges include incorporation of lactams and piperazinones (see, e.g. Giannis and Kolter, Angew. Chem. Int. Ed., 32:1244 (1993)).

As used herein in reference to a peptide, the term “cyclic” refers to a structure including an intramolecular bond between two non-adjacent amino acids or amino acid analogs. The cyclization can be achieved through a covalent or non-covalent bond. Intramolecular bonds include, but are not limited to, backbone to backbone, side-chain to backbone, side-chain to side-chain, side chain to end-group, and end-to-end bonds. Methods of cyclization include, without limitation, formation of a disulfide bond between the side-chains of non-adjacent amino acids or amino acid analogs; formation of an amide bond between the side-chains of Lys and Asp/Glu residues; formation of an ester bond between serine residues and Asp/Glu residues; formation of a lactam bond, for example, between a side-chain group of one amino acid or analog thereof to the N-terminal amine of the amino-terminal residue; and formation of lysinonorleucine and dityrosine bonds. Carbon versions of a disulfide linkage, for example an ethenyl or ethyl linkage, could also be used (J. Peptide Sc. 14:898-902 (2008)) as well as alkylation reactions with an appropriately polysubstituted electrophilic reagent such as a di-, tri- or tetrahaloalkane (PNAS, 105 (40), 15293-15298 (2008); ChemBioChem, 6:821-824 (2005)). Various modified proline analogs can also be used to incorporate conformational constraints into peptides (Zhang et al., J. Med. Chem., 39:2738-2744 (1996); Pfeifer and Robinson, Chem. Comm., 1977-1978 (1998)). Chemistries that may be used to cyclize peptides of the disclosure result in peptides cyclized with a bond including, but not limited to the following: lactam, hydrazone, oxime, thiazolidine, thioether or sulfonium bonds.

Yet another approach in the design of conformationally constrained peptides, which is described in US Patent Publication No. 2004-0176283, is to attach a short amino acid sequence of interest to a template to generate a cyclic constrained peptide. Such cyclic peptides are not only structurally stabilized by their templates, and thereby offer three-dimensional conformations that may imitate conformational epitopes on viruses and parasites, but they are also more resistant than linear peptides to proteolytic degradation in serum. US Patent Publication No. 2004-0176283 further discloses the synthesis of conformationally constrained cross-linked peptides by preparation of synthetic amino acids for backbone coupling to appropriately positioned amino acids in order to stabilize the supersecondary structure of peptides. Cross-linking can be achieved by amide coupling of the primary amino group of an orthogonally protected (2S,3R)-3-aminoproline residue to a suitably positioned side chain carboxyl group of glutamate. This approach has been followed in the preparation of conformationally constrained tetrapeptide repeats of the CS protein wherein at least one proline has been replaced by (2S,3R)-3-aminoproline and, in order to introduce a side chain carboxyl group, glutamate has been incorporated as a replacement for alanine.

Cross-linking strategies also include the application of the Grubbs ring-closing metathesis reaction to form ‘stapled’ peptides designed to mimic alpha-helical conformations (Angew. Int. Ed. Engl. 37:3281 (1998); JACS 122:5891 (2000)); use of poly-functionalized saccharides; use of a tryptathionine linkage (Chemistry Eu. J. 24:3404-3409 (2008)); and use of ‘click’ reaction of azides and alkynes which could be incorporated as either a side chain amino acid residue or located within the backbone of the peptide sequence (Drug Disc. Today 8 (24):1128-1137 (2003)). It is also known in the literature that metal ions can stabilize constrained conformations of linear peptides through sequestering specific residues (e.g. histidine) which coordinate to metal cations (Angew. Int. Ed. Engl. 42:421 (2003)). Similarly, functionalizing a linear peptide sequence with non-natural acid and amine functionality, or polyamine and polyacid functionality can be used to allow access to cyclized structures following activation and amide bond formation.

According to one embodiment, the antigenic tau peptide is conformationally constrained by intramolecular covalent bonding of two non-adjacent amino acids of the antigenic tau peptide to each other, e.g. the N- and C-terminal amino acids. According to another embodiment, the antigenic tau peptide of the disclosure is conformationally constrained by covalent binding to a scaffold molecule. According to a further embodiment, the antigenic tau peptide is simply constrained, i.e. coupled either at one end, (C or N terminus) or through another amino acid not located at either end, to the scaffold molecule. According to another embodiment, the antigenic tau peptide is doubly constrained, i.e. coupled at both C and N termini to the scaffold molecule.

The scaffold (also called ‘platform’) can be any molecule which is capable of reducing, through covalent bonding, the number of conformations which the antigenic tau peptide can assume. Examples of conformation-constraining scaffolds include proteins and peptides, for example lipocalin-related molecules such as beta-barrel containing thioredoxin and thioredoxin-like proteins, nucleases (e.g. RNaseA), proteases (e.g. trypsin), protease inhibitors (e.g. eglin C), antibodies or structurally-rigid fragments thereof, fluorescent proteins such as GFP or YFP, conotoxins, loop regions of fibronectin type III domain, CTLA-4, and virus-like particles (VLPs).

Other suitable platform molecules include carbohydrates such as sepharose. The platform may be a linear or circular molecule, for example, closed to form a loop. The platform is generally heterologous with respect to the antigenic tau peptide. Such conformationally constrained peptides linked to a platform are thought to be more resistant to proteolytic degradation than linear peptide.

According to one embodiment, the scaffold is an immunogenic carrier as defined in the present disclosure, such as a heterologous carrier protein or a VLP. In a further embodiment, the antigenic tau peptide is simply constrained onto the immunogenic carrier. In a further embodiment, the antigenic tau peptide is doubly constrained onto the immunogenic carrier. In this manner, the antigenic tau peptide forms a conformationally constrained loop structure which has proven to be a particularly suitable structure as an intracellular recognition molecule.

The antigenic tau peptides of the disclosure may be modified for the ease of conjugation to a platform, for example by the addition of a terminal cysteine at one or both ends and/or by the addition of a linker sequence, such as double glycine head or tail, a linker terminating with a lysine residue, or any other linker known to those skilled in the art to perform such function. Bioorthogonal chemistry (such as the click reaction described above) to couple the full peptide sequence to the carrier, thus avoiding any regiochemical and chemoselectivity issues, might also be used. Rigid linkers such as those described in Jones et al. (Angew. Chem. Int. Ed. 2002, 41:4241-4244) are known to elicit an improved immunological response and might also be used.

In a further embodiment, the antigenic tau peptide is attached to a multivalent template, which itself is coupled to the carrier, thus increasing the density of the antigen (see below). The multivalent template could be an appropriately functionalized polymer or oligomer such as (but not limited to) oligoglutamate or oligochitosan.

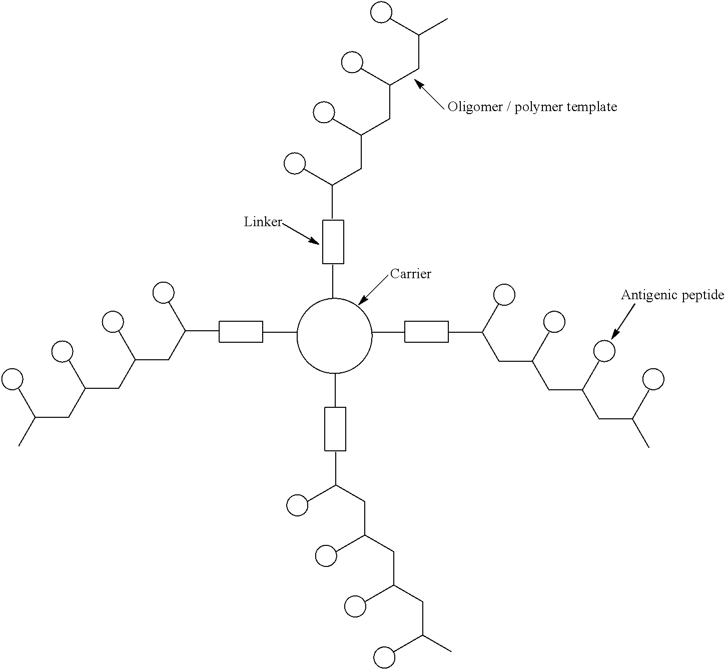

Said linker might be located at the N-terminus of the peptide, or at the C-terminus of the peptide, or both ends of the peptide. Said linker might be from 0 to 10 amino acids long, for example from 0 to 6 amino acids long. Alternatively, the addition or substitution of a D-stereoisomer form of one or more of the amino acids may be performed to create a beneficial derivative, for example to enhance stability of the peptide.

Examplary combinations of conjugations, all within the scope of the present disclosure and constituting various embodiments, using various linkers are provided below:

| | |
|---|---|
| Peptide-GGGGGC | (SEQ ID NO: 79)-scaffold; |
| Peptide-GGGGC | (SEQ ID NO: 80)-scaffold; |
| Peptide-GGGC | (SEQ ID NO: 81)-scaffold; |
| Peptide-GGC-scaffold; | |
| Peptide-GC-scaffold; | |
| Peptide-C-scaffold; | |
| Peptide-GGGGGK; | (SEQ ID NO: 82) |
| Peptide-GGGGK | (SEQ ID NO: 83) |
| Peptide-GGGK; | (SEQ ID NO: 84) |
| Peptide-GGK; | |
| Peptide-GK; | |
| Peptide-K; | |
| Peptide-GGGGSC; | (SEQ ID NO: 85) |
| Peptide-GGGSC; | (SEQ ID NO: 86) |
| Peptide-GGSC; | (SEQ ID NO: 87) |
| Peptide-GSC; | |
| Peptide-SC; | |
| Peptide-GGGGC; | (SEQ ID NO: 80) |
| Peptide-GGGC; | (SEQ ID NO: 81) |
| Peptide-GGC; | |
| Peptide-GC; | |
| CSGGGG | (SEQ ID NO: 88)-Peptide; |
| CSGGG | (SEQ ID NO: 89)-Peptide; |
| CSGG | (SEQ ID NO: 90)-Peptide; |
| CSG-Peptide; | |
| CS-Peptide; | |
| CGGGG | (SEQ ID NO: 91)-Peptide; |
| CGGG | (SEQ ID NO: 92)-Peptide; |
| CGG-Peptide; | |
| CG-Peptide | |

Examplary combinations of conjugations using various linkers and doubly constrained peptides are provided below, where the carrier can be the identical monomer of a carrier or a differential monomer of a carrier. In the example below, the GC linker can be substituted by any of the GK linker or GSC linker exemplified above or any other known to those skilled in the art:

| | |
|---|---|
| Carrier-CGGGGG | (SEQ ID NO: 93)-Peptide- |
| GGGGGC | (SEQ ID NO: 79)-carrier; |
| Carrier-CGGGG | (SEQ ID NO: 91)-Peptide- |
| GGGGC | (SEQ ID NO: 80)-carrier; |
| Carrier-CGGG | (SEQ ID NO: 92)-Peptide- |
| GGGC | (SEQ ID NO: 81)-carrier; |
| Carrier-CG- | Peptide- |
| GC-carrier; | |
| Carrier-C- | Peptide- |
| C-carrier | |

In one embodiment, a terminal cysteine residue, if not already present in the amino acid sequence of the antigenic tau peptide, is added to one or both ends of an antigenic tau peptide comprising or consisting of any of the sequences set forth in SEQ ID NOs: 1 to 26 to generate a conformationally constrained peptide.

In another embodiment, a GC linker comprising a variable number of glycine residues and one terminal cysteine residue is added to one or both ends of an antigenic tau peptide comprising or consisting of any of the sequences set forth in SEQ ID NOs: 1 to 26 to generate a conformationally constrained peptide. Preferably, the GC linker comprises from 1 to 10 glycine residues, more preferably 1, 2, 3, 4, or 5 glycine residues.

In yet another embodiment, a GC linker comprising a variable number of glycine residues and one terminal cysteine residue is added to one end of an antigenic tau peptide comprising or consisting of any of the sequences set forth in SEQ ID NOs: 1 to 26 and a terminal cysteine residue, if not already present to the other end of the antigenic tau peptide, is added to the other end of the antigenic peptide. Preferably, the GC linker comprises from 1 to 10 glycine residues, more preferably 1, 2, 3, 4, or 5 glycine residues.

### Immunogenic Carriers

In one embodiment of the present disclosure, the antigenic tau peptide or polypeptide of the disclosure is linked to an immunogenic carrier molecule to form immunogens for vaccination protocols. The term “immunogenic carrier” herein includes those materials which have the property of independently eliciting an immunogenic response in a host animal and which can be linked (e.g. covalently coupled) to a peptide, polypeptide or protein either directly via formation of peptide or ester bonds between free carboxyl, amino or hydroxyl groups in the peptide, polypeptide or protein and corresponding groups on the immunogenic carrier material, or alternatively by bonding through a conventional bifunctional linking group, or as a fusion protein.

The types of carriers used in the immunogens of the present disclosure will be readily known to those skilled in the art. Examples of such immunogenic carriers are: virus-like particles (VLP); serum albumins such as bovine serum albumin (BSA); globulins; thyroglobulins; hemoglobins; hemocyanins (particularly Keyhole Limpet Hemocyanin (KLH)); proteins extracted from ascaris, inactivated bacterial toxins or toxoids such as tetanus or diptheria toxins (TT and DT) or CRM197, the purified protein derivative of tuberculin (PPD); or Protein D from Haemophilus influenzae (PCT Publication No. WO 91/18926) or recombinant fragments thereof (for example, Domain 1 of Fragment C of TT, or the translocation domain of DT or Protein D 1/3rd comprising the N-terminal 100 to 110 amino acids of Haemophilus influenzae protein D (GB 9717953. 5); polylysin; polyglutamic acid; lysine-glutamic acid copolymers; copolymers containing lysine or ornithine; liposome carriers, etc.

In one embodiment, the immunogenic carrier is KLH. In another embodiment, the immunogenic carrier is a virus-like particle (VLP), preferably a recombinant virus-like particle.

The term “virus particle” as used herein refers to the morphological form of a virus. In some virus types it comprises a genome surrounded by a protein capsid; others have additional structures, such as envelopes, tails, etc.

The term “virus-like particle” (VLP), as used herein, refers to a non-replicative and/or noninfectious virus particle, or refers to a non-replicative and/or non-infectious structure resembling a virus particle, such as a capsid of a virus. The term “non-replicative”, as used herein, refers to the inability to replicate the genome comprised by the VLP. The term “non-infectious”, as used herein, refers the inability to enter a host cell. In one example, a virus-like particle is non-replicative and/or non-infectious since it lacks all or part of the viral genome or genome function. For example, a virus-like particle is a virus particle, in which the viral genome has been physically or chemically inactivated. Further, for example, a virus-like particle lacks all or part of the replicative and infectious components of the viral genome. A virus-like particle may contain nucleic acid distinct from the genome of the virus. One example of a virus-like particle is a viral capsid such as the viral capsid of the corresponding virus, for example a bacteriophage, such as RNA-phage. The terms “viral capsid” or “capsid”, refer to a macromolecular assembly composed of viral protein subunits. For example there can be 60, 120, 180, 240, 300, 360 and more than 360 viral protein subunits. The interactions of these subunits can lead to the formation of viral capsid or viral-capsid like structure with an inherent repetitive organization, wherein said structure is, for example, spherical or tubular.

As used herein, the term “virus-like particle of a RNA phage” refers to a virus-like particle comprising, or consisting essentially of, or consisting of, coat proteins, variants or fragments thereof, of a RNA phage. For example, a virus-like particle of a RNA phage can resemble the structure of a RNA phage, being non-replicative and/or non-infectious, and lacking at least the gene or genes encoding for the replication machinery of the RNA phage, and may also lack the gene or genes encoding the protein or proteins responsible for viral attachment to, or entry into, the host. This definition should, however, also encompass virus-like particles of RNA phages, in which the aforementioned gene or genes are still present but inactive, and, therefore, also leading to non-replicative and/or non-infectious virus-like particles of a RNA phage. Within the present disclosure, the term “subunit” and “monomer” are interchangeably and equivalently used within this context. Further, in the present disclosure, the term “RNA-phage” and the term “RNA-bacteriophage” are interchangeably used.

The present disclosure provides compositions and methods for inducing and/or enhancing immune responses against phosphorylated tau in a mammal. Compositions of the disclosure can comprise a virus-like particle (VLP) linked to at least one antigenic tau peptide. For example, an antigenic tau peptide can be linked to the VLP so as to form an ordered and repetitive antigen-VLP array. For example, in one case at least 20, at least 30, at least 60, at least 120, at least 180, at least 360, or at least 540 peptides as described herein are linked to the VLP.

The capsid structure formed from the self-assembly of 180 subunits of RNA phage coat protein and optionally containing host RNA is herein referred to as a “VLP of RNA phage coat protein”. A specific example is the VLP of Qbeta coat protein. In this particular case, the VLP of Qbeta coat protein may either be assembled exclusively from Qbeta CP subunits (generated by expression of a Qbeta CP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly. Generally, the percentage of Qbeta A1 protein relative to Qbeta CP in the capsid assembly will be limited, in order to ensure capsid formation.

Examples of VLPs suitable as immunogenic carriers in the context of the present disclosure include, but are not limited to, the capsid proteins of Hepatitis B virus (Ulrich, et al., Virus Res. 50: 141-182 (1998)), measles virus (Warnes, et al., Gene 160: 173-178 (1995)), Sindbis virus, rotavirus (U.S. Pat. Nos. 5,071,651 and 5,374,426), foot-and-mouth-disease virus (Twomey, et al., Vaccine 13: 1603-1610, (1995)), Norwalk virus (Jiang, X., et al., Science 250: 1580-1583 (1990); Matsui, S. M., et al., J. Clin. Invest. 87: 1456-1461 (1991)), the retroviral GAG protein (PCT Publication No. WO 96/30523), the retrotransposon Ty protein pl, the surface protein of Hepatitis B virus (PCT Publication No. WO 92/11291), human papilloma virus (PCT Publication No. WO 98/15631), human polyoma virus (Sasnauskas K., et al., Biol. Chem. 380 (3): 381-386 (1999); Sasnauskas K., et al., Generation of recombinant virus-like particles of different polyomaviruses in yeast, 3rd International Workshop “Virus-like particles as vaccines”, Berlin, Sep. 26-29 (2001)), RNA phages, Ty, frphage, GA-phage, AP 205-phage and, in particular, Qbeta-phage.

As will be readily apparent to those skilled in the art, the VLP to be used as an immunogenic carrier of the disclosure is not limited to any specific form. The particle can be synthesized chemically or through a biological process, which can be natural or normatural. By way of example, this type of embodiment includes a virus-like particle or a recombinant form thereof. In a more specific embodiment, the VLP can comprise, or alternatively consist of, recombinant polypeptides of any of the virus known to form a VLP. The VLP can further comprise, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts. Variant VLPs suitable for use in the present disclosure can be derived from any organism so long as they are able to form a “virus-like particle” and can be used as an “immunogenic carrier” as defined herein.

Preferred VLPs according to the disclosure include the capsid protein or core and surface antigen of HBV (HBcAg and HBsAg respectively) or recombinant proteins or fragments thereof, and the coat proteins of RNA-phages or recombinant proteins or fragments thereof, more preferably the coat protein of Qbeta or recombinant proteins or fragments thereof.

In one embodiment, the immunogenic carrier used in combination with an antigenic tau peptide of the disclosure is an HBcAg protein. Examples of HBcAg proteins that can be used in the context of the present disclosure can be readily determined by one skilled in the art. Examples include, but are limited to, HBV core proteins described in Yuan et al., J. Virol. 73:10122-10128 (1999), and in PCT Publication Nos. WO 00/198333, WO 00/177158, WO 00/214478, WO 00/32227, WO 01/85208, WO 02/056905, WO 03/024480, and WO 03/024481. HBcAgs suitable for use in the present disclosure can be derived from any organism so long as they are able to form a “virus-like particle” and can be used as an “immunogenic carrier” as defined herein.

HBcAg variants of particular interest that can be used in the context of the present disclosure are those variants in which one or more naturally occurring cysteine residues have been either deleted or substituted. It is well known in the art that free cysteine residues can be involved in a number of chemical side reactions including disulfide exchanges, reaction with chemical substances or metabolites that are, for example, injected or formed in a combination therapy with other substances, or direct oxidation and reaction with nucleotides upon exposure to UV light. Toxic adducts could thus be generated, especially considering the fact that HBcAgs have a strong tendency to bind nucleic acids. The toxic adducts would thus be distributed between a multiplicity of species, which individually may each be present at low concentration, but reach toxic levels when together. In view of the above, one advantage to the use of HBcAgs in vaccine compositions which have been modified to remove naturally occurring cysteine residues is that sites to which toxic species can bind when antigens or antigenic determinants are attached would be reduced in number or eliminated altogether.

In addition, the processed form of HBcAg lacking the N-terminal leader sequence of the Hepatitis B core antigen precursor protein can also be used in the context of the disclosure, especially when HBcAg is produced under conditions where processing will not occur (e.g. expression in bacterial systems).

Other HBcAg variants according to the disclosure include i) polypeptide sequence that are at least 80%, 85%, 90%, 95%, 97% or 99% identical to one of the wild-type HBcAg amino acid sequences, or a subportion thereof, using standard sequence comparison computer algorithms, ii) C-terminal truncation mutants including mutants where at least 1, 5, 10, 15, 20, 25, 30, 34, or 35 amino acids have been removed from the C-terminus, ii) N-terminal truncation mutants including mutants where at least 1, 2, 5, 7, 9, 10, 12, 14, 15, or 17 amino acids have been removed from the N-terminus, iii) mutants truncated in both N-terminal and C-terminal including HBcAgs where at least 1, 2, 5, 7, 9, 10, 12, 14, 15, or 17 amino acids have been removed from the N-terminus and at least 1, 5, 10, 15, 20, 25, 30, 34, or 35 amino acids have been removed from the C-terminus.

Still other HBcAg variant proteins within the scope of the disclosure are those variants modified in order to enhance immunogenic presentation of a foreign epitope wherein one or more of the four arginine repeats has been deleted, but in which the C-terminal cysteine is retained (see e.g. PCT Publication No. WO 01/98333), and chimeric C-terminally truncated HBcAg such as those described in PCT Publication Nos. WO 02/14478, WO 03/102165 and WO 04/053091.

In another embodiment, the immunogenic carrier used in combination with an antigenic tau peptide of the disclosure is an HBsAg protein. HBsAg proteins that can be used in the context of the present disclosure can be readily determined by one skilled in the art. Examples include, but are not limited to, HBV surface proteins described in U.S. Pat. No. 5,792,463, and PCT Publication Nos. WO 02/10416, and WO 08/020,331. HBsAgs suitable for use in the present disclosure can be derived from any organism so long as they are able to form a “virus-like particle” and can be used as an “immunogenic carrier” as defined herein.

In still another embodiment, the immunogenic carrier used in combination with an antigenic tau peptide of the disclosure is a Qbeta coat protein. Qbeta coat protein was found to self-assemble into capsids when expressed in E. coli (Kozlovska T. M. et al., GENE 137: 133-137 (1993)). The obtained capsids or virus-like particles showed an icosahedral phage-like capsid structure with a diameter of 25 nm and T=3 quasi symmetry. Further, the crystal structure of bacteriophage Qbeta has been solved. The capsid contains 180 copies of the coat protein, which are linked in covalent pentamers and hexamers by disulfide bridges (Golmohammadi, R. et al., Structure 4: 5435554 (1996)) leading to a remarkable stability of the capsid of Qbeta coat protein. Qbeta capsid protein also shows unusual resistance to organic solvents and denaturing agents. The high stability of the capsid of Qbeta coat protein is an advantageous feature, in particular, for its use in immunization and vaccination of mammals and humans in the context of the present disclosure

Examples of Qbeta coat proteins that can be used in the context of the present disclosure can be readily determined by one skilled in the art. Examples have been extensively described in PCT Publication Nos. WO 02/056905, WO 03/024480, WO 03/024481 and include, but are not limited to, amino acid sequences disclosed in the PIR database, Accession No. VCBPQbeta referring to Qbeta CP; Accession No. AAA16663 referring to Qbeta A1 protein; and variants thereof including variant proteins in which the N-terminal methionine is cleaved; C-terminal truncated forms of Qbeta A1 missing as much as 100, 150 or 180 amino acids; variant proteins which have been modified by the removal of a lysine residue by deletion or substitution or by the addition of a lysine residue by substitution or insertion (see for example Qbeta-240, Qbeta-243, Qbeta-250, Qbeta-251 and Qbeta-259 disclosed in PCT Publication No. WO 03/024481), and variants exhibiting at least 80%, 85%, 90%, 95%, 97%, or 99% identity to any of the Qbeta core proteins described herein. Variant Qbeta coat proteins suitable for use in the present disclosure can be derived from any organism so long as they are able to form a “virus-like particle” and can be used as “immunogenic carriers” as defined herein.

### Linkage

The antigenic tau peptides of the disclosure may be coupled to immunogenic carriers via chemical conjugation or by expression of genetically engineered fusion partners. The coupling does not necessarily need to be direct, but can occur through linker sequences. More generally, in the case where antigenic peptides are fused, conjugated or otherwise attached to an immunogenic carrier, spacer or linker sequences are typically added at one or both ends of the antigenic peptides. Such linker sequences generally comprise sequences recognized by the proteasome, proteases of the endosomes or other vesicular compartment of the cell.

In one embodiment, the peptides of the present disclosure are expressed as fusion proteins with the immunogenic carrier. Fusion of the peptide can be effected by insertion into the immunogenic carrier primary sequence, or by fusion to either the N- or C-terminus of the immunogenic carrier. Hereinafter, when referring to fusion proteins of a peptide to an immunogenic carrier, the fusion to either ends of the subunit sequence or internal insertion of the peptide within the carrier sequence are encompassed. Fusion, as referred to hereinafter, may be carried out by insertion of the antigenic peptide into the sequence of the carrier, by substitution of part of the sequence of the carrier with the antigenic peptide, or by a combination of deletion, substitution or insertions.

When the immunogenic carrier is a VLP, the chimeric antigenic peptide-VLP subunit will be in general capable of self-assembly into a VLP. VLP displaying epitopes fused to their subunits are also herein referred to as chimeric VLPs. For example, EP 0 421 635 B describes the use of chimeric hepadnavirus core antigen particles to present foreign peptide sequences in a virus-like particle.

Flanking amino acid residues may be added to either end of the sequence of the antigenic peptide to be fused to either end of the sequence of the subunit of a VLP, or for internal insertion of such peptidic sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

In a specific embodiment of the disclosure, the immunogenic carrier is an HBcAg VLP. Fusion proteins of the antigenic peptide to either the N-terminus of HBcAg (Neyrinck, S. et al., Nature Med. 5:11571163 (1999)) or insertions in the so called major immunodominant region (MIR) have been described (Pumpens et al., Intervirology 44:98-114 (2001), PCT Publication No. WO 01/98333), and are specific embodiments of the disclosure. Naturally occurring variants of HBcAg with deletions in the MIR have also been described (Pumpens et al., Intervirology 44:98-114 (2001)), and fusions to the N- or C-terminus, as well as insertions at the position of the MIR corresponding to the site of deletion as compared to a wt HBcAg are further embodiments of the disclosure. Fusions to the C-terminus have also been described (Pumpens et al., Intervirology 44:98-114 (2001)). One skilled in the art will easily find guidance on how to construct fusion proteins using classical molecular biology techniques. Vectors and plasmids encoding HBcAg and HBcAg fusion proteins and useful for the expression of a HBcAg and HBcAg fusion proteins have been described (Pumpens et al., Intervirology 44:98-114 (2001), Neyrinck, S. et al., Nature Med. 5:1157-1163 (1999)) and can be used in the practice of this disclosure. An important factor for the optimization of the efficiency of self-assembly and of the display of the epitope to be inserted in the MIR of HBcAg is the choice of the insertion site, as well as the number of amino acids to be deleted from the HBcAg sequence within the MIR (European Patent No. EP 0421635; U.S. Pat. No. 6,231,864) upon insertion, or in other words, which amino acids form HBcAg are to be substituted with the new epitope. For example, substitution of HBcAg amino acids 76-80, 79-81, 79-80, 75-85 or 80-81 with foreign epitopes has been described (Pumpens et al., Intervirology 44:98-114 (2001); European Patent No. EP 0421635; U.S. Pat. No. 6,231,864, PCT Patent Publication No. WO00/26385). HBcAg contains a long arginine tail that is dispensable for capsid assembly and capable of binding nucleic acids. HBcAg either comprising or lacking this arginine tail are both embodiments of the present disclosure.

In another specific embodiment of the disclosure, the immunogenic carrier is a VLP of a RNA phage, preferably Qbeta. The major coat proteins of RNA phages spontaneously assemble into VLPs upon expression in bacteria, and in particular in E. coli. Fusion protein constructs wherein antigenic peptides have been fused to the C-terminus of a truncated form of the A1 protein of Qbeta, or inserted within the A1 protein have been described (Kozlovska et al., Intervirology, 39:9-15 (1996)). The A1 protein is generated by suppression at the UGA stop codon and has a length of 329 amino acids, or 328 amino acids, if the cleavage of the N-terminal methionine is taken into account. Cleavage of the N-terminal methionine before an alanine (the second amino acid encoded by the Qbeta CP gene) usually takes place in E. coli, and such is the case for N-termini of the Qbeta coat proteins. The part of the A1 gene, 3′ of the UGA amber codon encodes the CP extension, which has a length of 195 amino acids. Insertion of the antigenic peptide between position 72 and 73 of the CP extension leads to further embodiments of the disclosure (Kozlovska et al., Intervirology 39:9-15 (1996)). Fusion of an antigenic peptide at the C-terminus of a C-terminally truncated Qbeta A1 protein leads to further preferred embodiments of the disclosure. For example, Kozlovska et al., Intervirology, 39:9-15 (1996) describe Qbeta A1 protein fusions where the epitope is fused at the C-terminus of the Qbeta CP extension truncated at position 19.

As described by Kozlovska et al., Intervirology, 39:9-15 (1996), assembly of the particles displaying the fused epitopes typically requires the presence of both the A1 protein-antigen fusion and the wild-type CP to form a mosaic particle. However, embodiments comprising virus-like particles, and hereby in particular the VLPs of the RNA phage Qbeta coat protein, which are exclusively composed of VLP subunits having an antigenic peptide fused thereto, are also within the scope of the present disclosure.

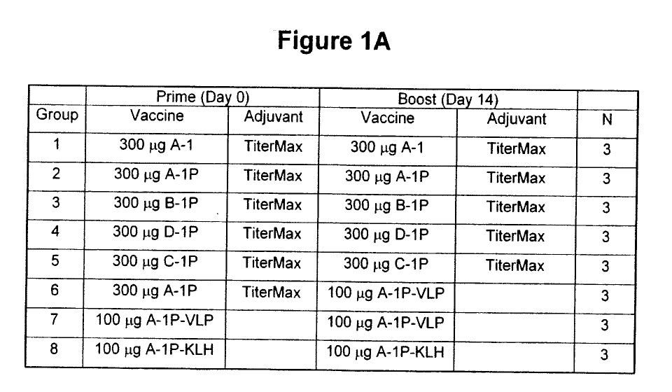

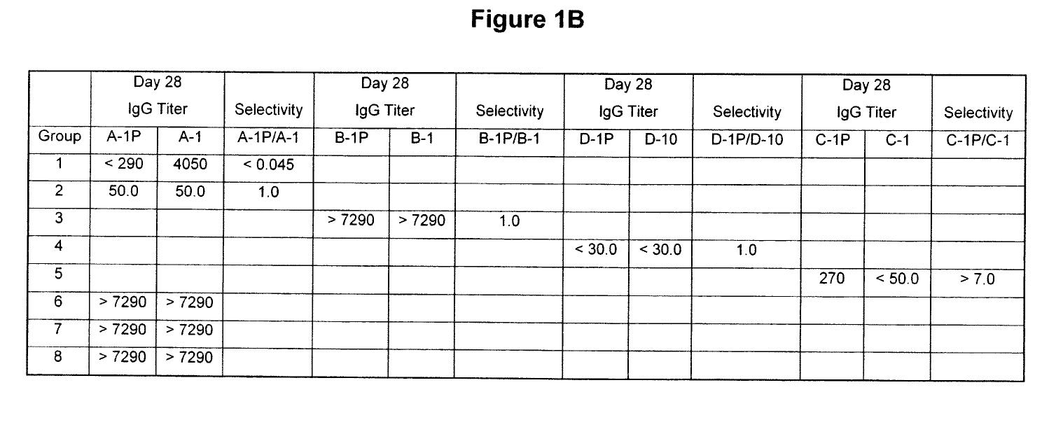

The production of mosaic particles may be carried out in a number of ways. Kozlovska et al., Intervirology 39:9-15 (1996), describe three methods, which all can be used in the practice of the disclosure. In the first approach, efficient display of the fused epitope on the VLPs is mediated by the expression of the plasmid encoding the Qbeta A1 protein fusion having a UGA stop codon between CP and CP extension in an E. coli strain harboring a plasmid encoding a cloned UGA suppressor tRNA which leads to translation of the UGA codon into Trp (pISM3001 plasmid (Smiley et al., Gene 134:33-40 (1993)). In another approach, the CP gene stop codon is modified to UAA, and a second plasmid expressing the A1 protein-antigen fusion is co-transformed. The second plasmid encodes a different antibiotic resistance and the origin of replication is compatible with the first plasmid. In a third approach, CP and the A1 protein-antigen fusion are encoded in a bicistronic manner, operatively linked to a promoter such as the Trp promoter, as described in FIG. 1 of Kozlovska et al., Intervirology, 39:9-15 (1996).

Further VLPs suitable for fusion of antigens or antigenic determinants are described in PCT Publication No. WO 03/024481 and include bacteriophage fr, RNA phase MS-2, capsid protein of papillomavirus, retrotransposon Ty, yeast and also Retrovirus-like particles, HIV2 Gag, Cowpea Mosaic Virus, parvovirus VP2 VLP, HBsAg (U.S. Pat. No. 4,722,840 and European Patent No. EP 0020416B1). Examples of chimeric VLPs suitable for the practice of the disclosure are also those described in Intervirology 39:1 (1996). Further examples of VLPs contemplated for use in the present disclosure are: HPV-1, HPV-6, HPV-11, HPV-16, HPV-18, HPV-33, HPV-45, CRPV, CPOV, HIV GAG, and Tobacco Mosaic Virus. Further examples include VLPs of SV-40, Polyomavirus, Adenovirus, Herpes Simplex Virus, Rotavirus, and Norwalk virus.

For any recombinantly expressed peptide or protein which forms part of the present disclosure, including an antigenic tau peptide according to the disclosure coupled or not to an immunogenic carrier, the nucleic acid which encodes said peptide or protein also forms an aspect of the present disclosure, as does an expression vector comprising the nucleic acid, and a host cell containing the expression vector (autonomously or chromosomally inserted). A method of recombinantly producing the peptide or protein by expressing it in the above host cell and isolating the immunogen therefrom is a further aspect of the disclosure.

In another embodiment, the peptide of the disclosure is chemically coupled to an immunogenic carrier, using techniques well known in the art. Conjugation can occur to allow free movement of peptides via single point conjugation (e.g. either N-terminal or C-terminal point) or as locked down structure where both ends of peptides are conjugated to either an immunogenic carrier protein or to a scaffold structure such as a VLP. Such conjugation can be carried out via conjugation chemistry known to those skilled in the art such as via cysteine residues, lysine residues or other carboxy moieties commonly known as conjugation points such as glutamic acid or aspartic acid. Thus, for example, for direct covalent coupling it is possible to utilize a carbodiimide, glutaraldehyde or (N-[y-malcimidobutyryloxy]succinimide ester, utilizing common commercially available heterobifunctional linkers such as CDAP and SPDP (using manufacturer's instructions). Examples of conjugation of peptides, particularly cyclized peptides, to a protein carrier via acylhydrazine peptide derivatives are described in PCT Publication No. WO 03/092714. After the coupling reaction, the immunogen can easily be isolated and purified by means of a dialysis method, a gel filtration method, a fractionation method etc. Peptides terminating with a cysteine residue (preferably with a linker outside the cyclized region) may be conveniently conjugated to a carrier protein via maleimide chemistry.

When the immunogenic carrier is a VLP, several antigenic peptides, either having an identical amino acid sequence or a different amino acid sequence, may be coupled to a single VLP molecule, leading preferably to a repetitive and ordered structure presenting several antigenic determinants in an oriented manner as described in PCT Publication Nos. WO 00/32227, WO 03/024481, WO 02/056905 and WO 04/007538.

In one aspect of the disclosure, the antigenic peptide is bound to the VLP by way of chemical cross-linking, typically and preferably by using a heterobifunctional cross-linker. Several hetero-bifunctional cross-linkers are known in the art. In some embodiments, the hetero-bifunctional crosslinker contains a functional group which can react with first attachment sites, i.e. with the side-chain amino group of lysine residues of the VLP or VLP subunit, and a further functional group which can react with a preferred second attachment site, i.e. a cysteine residue fused to the antigenic peptide and optionally also made available for reaction by reduction. The first step of the procedure, typically called the derivatization, is the reaction of the VLP with the cross-linker. The product of this reaction is an activated VLP, also called activated carrier. In the second step, unreacted cross-linker is removed using standard methods such as gel filtration or dialysis. In the third step, the antigenic peptide is reacted with the activated VLP, and this step is typically called the coupling step. Unreacted antigenic peptide may be optionally removed in a fourth step, for example by dialysis. Several hetero-bifunctional crosslinkers are known in the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive toward cysteine residues. The above mentioned cross-linkers all lead to formation of a thioether linkage.

Another class of cross-linkers suitable in the practice of the disclosure is characterised by the introduction of a disulfide linkage between the antigenic peptide and the VLP upon coupling. Preferred cross-linkers belonging to this class include for example SPDP and Sulfo-LC-SPDP (Pierce). The extent of derivatization of the VLP with cross-linker can be influenced by varying experimental conditions such as the concentration of each of the reaction partners, the excess of one reagent over the other, the pH, the temperature and the ionic strength. The degree of coupling, i.e. the amount of antigenic peptide per subunits of the VLP can be adjusted by varying the experimental conditions described above to match the requirements of the vaccine.

Another method of binding of antigenic peptides to the VLP is the linking of a lysine residue on the surface of the VLP with a cysteine residue on the antigenic peptide. In some embodiments, fusion of an amino acid linker containing a cysteine residue, as a second attachment site or as a part thereof, to the antigenic peptide for coupling to the VLP may be required. In general, flexible amino acid linkers are favored. Examples of the amino acid linker are selected from the group consisting of: (a) CGG; (b) N-terminal gamma 1-linker; (c) N-terminal gamma 3-linker; (d) Ig hinge regions; (e) N-terminal glycine linkers; (f) (G)ₖC(G)ₙ with n=0 to 12 and k=0 to 5; (g) N-terminal glycine-serine linkers; (h) (G)ₖC(G)ₘ(S)ᵢ(GGGGS)ₙ with n=0 to 3, k=0 to 5, m=0 to 10, i=0 to 2; (i) GGC; (k) GGC-NH2; (I) C-terminal gamma 1-linker; (m) C-terminal gamma 3-linker; (n) C-terminal glycine linkers; (O) (G)ₙC(G)ₖ with n=0 to 12 and k=0 to 5; (p) C-terminal glycine-serine linkers; (q) (G)ₘ(S)ₜ(GGGGS)ₙ(G)ₒC(G)ₖ with n=0 to 3, k=0 to 5, m=0 to 10, t=0 to 2, and o=0 to 8. Further examples of amino acid linkers are the hinge region of immunoglobulins, glycine serine linkers (GGGGS)ₙ, and glycine linkers (G)ₙ all further containing a cysteine residue as a second attachment site and optionally further glycine residues. Typically preferred examples of said amino acid linkers are N-terminal gamma 1: CGDKTHTSPP (SEQ ID NO:94); C-terminal gamma 1: DKTHTSPPCG (SEQ ID NO:95); N-terminal gamma 3: CGGPKPSTPPGSSGGAP (SEQ ID NO:96); C-terminal gamma 3: PKPSTPPGSSGGAPGGCG (SEQ ID NO:97); N-terminal glycine linker: GCGGGG (SEQ ID NO:98) and C-terminal glycine linker: GGGGCG (SEQ ID NO:99).

Other amino acid linkers particularly suitable in the practice of the disclosure, when a hydrophobic antigenic peptide is bound to a VLP, are CGKKGG (SEQ ID NO: 100), or CGDEGG (SEQ ID NO: 101) for N-terminal linkers, or GGKKGC (SEQ ID NO: 102) and GGEDGC (SEQ ID NO: 103), for the C-terminal linkers. For the C-terminal linkers, the terminal cysteine is optionally C-terminally amidated.

In some embodiments of the present disclosure, GGCG (SEQ ID NO: 104), GGC or GGC-NH2 (“NH2” stands for amidation) linkers at the C-terminus of the peptide or CGG at its N-terminus are preferred as amino acid linkers. In general, glycine residues will be inserted between bulky amino acids and the cysteine to be used as a second attachment site to avoid potential steric hindrance of the bulkier amino acid in the coupling reaction. In a further embodiment of the disclosure, the amino acid linker GGC-NH2 is fused to the C-terminus of the antigenic peptide.

The cysteine residue present on the antigenic peptide is preferably in its reduced state to react with the hetero-bifunctional cross-linker on the activated VLP, that is a free cysteine or a cysteine residue with a free sulfhydryl group should be available. In the case where the cysteine residue functions as a binding site in an oxidized form, for example if it is forming a disulfide bridge, reduction of this disulfide bridge with e.g. DTT, TCEP or p-mercaptoethanol is preferred. Low concentrations of reducing agent are compatible with coupling as described in PCT Publication No. WO 02/05690, whereas higher concentrations inhibit the coupling reaction, as a skilled artisan would know, in which case the reductant should be removed or its concentration decreased prior to coupling, e.g. by dialysis, gel filtration or reverse phase HPLC.

Binding of the antigenic peptide to the VLP by using a hetero-bifunctional cross-linker according to the methods described above allows coupling of the antigenic peptide to the VLP in an oriented fashion. Other methods of binding the antigenic peptide to the VLP include methods wherein the antigenic peptide is cross-linked to the VLP using the carbodiimide EDC, and NHS.

In other methods, the antigenic peptide is attached to the VLP using a homo-bifunctional cross-linker such as glutaraldehyde, DSGBM [PEO] 4, BS3, (Pierce Chemical Company, Rockford, Ill., USA) or other known homo-bifunctional cross-linkers with functional groups reactive toward amine groups or carboxyl groups of the VLP.

Other methods of binding the VLP to an antigenic peptide include methods where the VLP is biotinylated, and the antigenic peptide expressed as a streptavidin-fusion protein, or methods wherein both the antigenic peptide and the VLP are biotinylated, for example as described in PCT Publication No. WO 00/23955. In this case, the antigenic peptide may be first bound to streptavidin or avidin by adjusting the ratio of antigenic peptide to streptavidin such that free binding sites are still available for binding of the VLP, which is added in the next step. Alternatively, all components may be mixed in a “one pot” reaction. Other ligand-receptor pairs, where a soluble form of the receptor and of the ligand is available, and are capable of being cross-linked to the VLP or the antigenic peptide, may be used as binding agents for binding antigenic peptide to the VLP. Alternatively, either the ligand or the receptor may be fused to the antigenic peptide, and so mediate binding to the VLP chemically bound or fused either to the receptor, or the ligand respectively. Fusion may also be effected by insertion or substitution.

One or several antigen molecules can be attached to one subunit of the capsid or VLP of RNA phage coat proteins, preferably through the exposed lysine residues of the VLP of RNA phages, if sterically allowable. A specific feature of the VLP of the coat protein of RNA phages and in particular of the Qbeta coat protein VLP is thus the possibility to couple several antigens per subunit. This allows for the generation of a dense antigen array.

In one embodiment of the disclosure, the binding and attachment, respectively, of the at least one antigen or antigenic determinant to the virus-like particle is by way of interaction and association, respectively, between at least one first attachment site of the virus-like particle and at least one second attachment of the antigenic peptide.

VLPs or capsids of Qbeta coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In a further embodiment of the present disclosure, the first attachment site is a lysine residue and/or the second attachment comprises a sulfhydryl group or a cysteine residue. In an even further embodiment of the present disclosure, the first attachment site is a lysine residue and the second attachment site is a cysteine residue. In further embodiments, the antigen or antigenic determinant is bound via a cysteine residue, to lysine residues of the VLP of RNA phage coat protein, and in particular to the VLP of Qbeta coat protein.

Another advantage of the VLPs derived from RNA phages is their high expression yield in bacteria that allows production of large quantities of material at affordable cost. Moreover, the use of the VLPs as carriers allow for the formation of robust antigen arrays and conjugates, respectively, with variable antigen density. In particular, the use of VLPs of RNA phages, and hereby in particular the use of the VLP of RNA phage Qbeta coat protein allows one to achieve very high epitope density.

In some embodiments, immunogenic compositions may comprise mixtures of immunogenic conjugates, i.e. immunogenic carriers coupled to one or several antigenic tau peptides. Thus, these immunogenic compositions may be composed of immunogenic carriers which differ in amino acid sequence. For example, vaccine compositions could be prepared comprising a “wild-type” VLP and a modified VLP protein in which one or more amino acid residues have been altered (e.g., deleted, inserted or substituted). Alternatively, the same immunogenic carrier might be used but coupled to antigenic tau peptides of different amino acid sequences.

The present disclosure therefore also relates to methods for producing an immunogen comprising: i) providing an antigenic tau peptide according to the disclosure, ii) providing an immunogenic carrier according to the disclosure, preferably a VLP, and iii) combining said antigenic tau peptide and said immunogenic carrier. In one embodiment, said combining step occurs through chemical cross-linking, preferably through a heterobifunctional cross-linker.

### Compositions Comprising an Antigenic tau Peptide

The present disclosure also relates to compositions, particularly immunogenic compositions also referred to as “subject immunogenic compositions”, comprising an antigenic tau peptide of the disclosure, preferably linked to an immunogenic carrier, more preferably a VLP, even more preferably an HBsAg, HBcAg or Qbeta VLP, and optionally at least one adjuvant. Such immunogenic compositions, particularly when formulated as pharmaceutical compositions, are deemed useful to prevent, treat or alleviate tau-related disorders, such as Alzheimer's disease.

### Immunogenic Compositions

In some embodiments, a subject immunogenic composition according to the disclosure comprises an antigenic tau peptide comprising an amino acid sequence selected from SEQ ID NOs: 1 to 26, 31 to 76. and 105 to 122. In some embodiments, said antigenic tau peptide is linked to an immunogenic carrier, preferably a VLP, more preferably to an HBsAg, HBcAg or Qbeta VLP.

A subject immunogenic composition comprising an antigenic tau peptide according to the disclosure can be formulated in a number of ways, as described in more detail below.

In some embodiments, a subject immunogenic composition comprises a single species of antigenic tau peptide, e.g., the immunogenic composition comprises a population of antigenic tau peptides, substantially all of which have the same amino acid sequence. In other embodiments, a subject immunogenic composition comprises two or more different antigenic tau peptides, e.g., the immunogenic composition comprises a population of antigenic tau peptides, the members of which population can differ in amino acid sequence.

For example, in some embodiments, a subject immunogenic composition comprises a first antigenic tau peptide, preferably linked to an immunogenic carrier, more preferably to a VLP, even more preferably to a HBsAg, HBcAg or Qbeta VLP, and comprising a first amino acid sequence selected from SEQ ID NOs: 1 to 26, 31 to 76, and 105-122; and at least a second antigenic tau peptide, preferably linked to an immunogenic carrier, more preferably to a VLP, even more preferably to a HBsAg, HBcAg or Qbeta VLP, and comprising a second amino acid sequence, preferably selected from SEQ ID NOs: 1 to 26, 31 to 76, and 105-122 where the second amino acid sequence differs from the first amino acid sequence by at least 1, 2, 3, 4, 5, 6 to 10, or 15 amino acids.

As another example, a subject immunogenic composition comprises a first antigenic tau peptide, preferably linked to an immunogenic carrier, more preferably to a VLP, even more preferably to a HBsAg, HBcAg or Qbeta VLP, and comprising a first amino acid sequence selected from SEQ ID NOs: 1 to 26, 31 to 76, and 105-122; a second antigenic tau peptide, preferably linked to an immunogenic carrier, more preferably to a VLP, even more preferably to an HBsAg, HBcAg or Qbeta VLP, and comprising a second amino acid sequence, preferably selected from SEQ ID NOs: 1 to 26, 31 to 76, and 105-122 where the second amino acid sequence differs from the first amino acid sequence by at least 1, 2, 3, 4, 5, 6 to 10, or 15 amino acids; and at least a third antigenic tau peptide, preferably linked to an immunogenic carrier, more preferably to a VLP, even more preferably to a HBsAg, HBcAg or Qbeta VLP, and comprising a third amino acid sequence, preferably selected from SEQ ID NOs: 1 to 26, 31 to 76, and 105-122 where the third amino acid sequence differs from both the first and the second amino acid sequences by at least 1, 2, 3, 4, 5, 6 to 10, or 15 amino acids.

In other embodiments, a subject immunogenic composition comprises a multimerized antigenic tau peptide, as described above. As used herein, the terms “immunogenic composition comprising an antigenic tau peptide” or “immunogenic composition of the disclosure” or “subject immunogenic composition” refers to an immunogenic composition comprising either single species (multimerized or not) or multiple species of antigenic tau peptide(s) coupled or not to an immunogenic carrier.

### Adjuvants

In some embodiments, a subject immunogenic composition comprises at least one adjuvant. Suitable adjuvants include those suitable for use in mammals, preferably in humans. Examples of known suitable adjuvants that can be used in humans include, but are not necessarily limited to, alum, aluminum phosphate, aluminum hydroxide, MF59™ (4.3% w/v squalene, 0.5% w/v polysorbate 80 (Tween 80), 0.5% w/v sorbitan trioleate (Span 85)), CpG-containing nucleic acids (where the cytosine is unmethylated), QS21 (saponin adjuvant), MPL (Monophosphoryl Lipid A), 3DMPL (3-O-deacylated MPL), extracts from Aquilla, ISCOMS (see, e.g., Sjölander et al., J. Leukocyte Biol. 64:713 (1998); PCT Publication Nos. WO 90/03184, WO 96/11711, WO 00/48630, WO 98/36772, WO 00/41720, WO 06/134423 and WO 07/026,190), LT/CT mutants, poly(D,L-lactide-co-glycolide) (PLG) microparticles, Quil A, interleukins, and the like. For veterinary applications including but not limited to animal experimentation, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (PCT Publication No. WO 90/14837; Chapter 10 in Vaccine design: the subunit and adjuvant approach, eds. Powell & Newman, Plenum Press 1995), containing 5% Squalene, 0.5% Tween 80 (polyoxyethylene sorbitan mono-oleate), and 0.5% Span 85 (sorbitan trioleate) (optionally containing muramyl tri-peptide covalently linked to dipalmitoyl phosphatidylethanolamine (MTP-PE)) formulated into submicron particles using a microfluidizer, (b) SAF, containing 10% Squalene, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, Mass.), Abisco® (Isconova, Sweden), or Iscomatrix® (Commonwealth Serum Laboratories, Australia), may be used or particles generated therefrom such as ISCOMs (immunostimulating complexes), which ISCOMS may be devoid of additional detergent e.g. PCT Publication No. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (PCT Publication No. WO 99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. Great Britain Patent No. GB-2220221, and European Patent No. EP-A-0689454, optionally in the substantial absence of alum when used with pneumococcal saccharides e.g. PCT Publication No. WO 00/56358; (6) combinations of 3dMPL with, for example, QS21 and/or oil-in-water emulsions e.g. EP-A-0835318, EP-A-0735898, EP-A-0761231; (7) oligonucleotides comprising CpG motifs [Krieg, Vaccine (2000) 19:618-622; Krieg, Curr Opin Mol Ther (2001) 3:15-24; Roman et al., Nat. Med. (1997) 3:849-854; Weiner et al., PNAS USA (1997) 94:10833-10837; Davis et al, J. Immunol (1998) 160:870-876; Chu et al., J. Exp. Med (1997) 186:1623-1631; Lipford et al, Ear. J. Immunol. (1997) 27:2340-2344; Moldoveami el al., Vaccine (1988) 16:1216-1224, Krieg et al., Nature (1995) 374:546-549; Klinman et al., PNAS USA (1996) 93:2879-2883; Ballas et al, J. Immunol, (1996) 157:1840-1845; Cowdery et al, J. Immunol (1996) 156:4570-4575; Halpern et al, Cell Immunol. (1996) 167:72-78; Yamamoto et al, Jpn. J. Cancer Res., (1988) 79:866-873; Stacey et al, J. Immunol., (1996) 157:2116-2122; Messina et al, J. Immunol, (1991) 147:1759-1764; Yi et al, J. Immunol. (1996) 157:4918-4925; Yi et al, J. Immunol (1996) 157:5394-5402; Yi et al, J. Immunol, (1998) 160:4755-4761; and Yi et al, J. Immunol, (1998) 160:5898-5906; PCT Publication Nos. WO 96/02555, WO 98/16247, WO 98/18810, WO 98/40100, WO 98/55495, WO 98/37919 and WO 98/52581] i.e. containing at least one CG dinucleotide, where the cytosine is unmethylated; (8) a polyoxyethylene ether or a polyoxyethylene ester e.g. PCT Publication No. WO 99/52549; (9) a polyoxyethylene sorbitan ester surfactant in combination with an octoxynol (PCT Publication No. WO 01/21207) or a polyoxyethylene alkyl ether or ester surfactant in combination with at least one additional non-ionic surfactant such as an octoxynol (PCT Publication No. WO 01/21152); (10) a saponin and an immunostimulatory oligonucleotide (e.g. a CpG oligonucleotide) (PCT Publication No. WO 00/62800); (11) an immunostimulant and a particle of metal salt e.g. PCT Publication No. WO 00/23105; (12) a saponin and an oil-in-water emulsion e.g. PCT Publication No. WO 99/11241; (13) a saponin (e.g. QS21)+3dMPL+IM2 (optionally+a sterol) e.g. PCT Publication No. WO 98/57659; (14) other substances that act as immunostimulating agents to enhance the efficacy of the composition, such as Muramyl peptides including N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), (15) ligands for toll-like receptors (TLR), natural or synthesized (e.g. as described in Kanzler et al., Nature Med. 13:1552-1559 (2007)), including TLR3 ligands such as polyl:C and similar compounds such as Hiltonol and Ampligen.

In one embodiment, the immunogenic composition of the present disclosure comprises at least one adjuvant. In a particular embodiment, said adjuvant is an immunostimulatory oligonucleotide and more preferably a CpG oligonucleotide. In one embodiment, the CpG oligonucleotide has the nucleic acid sequence 5′ TCGTCGTTTTGTCGTTTTGTCGTT 3′ (CpG 7909; SEQ ID NO:27). In another embodiment, the CpG oligonucleotide has the nucleic acid sequence 5′ TCGTCGTTTTTCGGTGCTTTT 3′ (CpG 24555; SEQ ID NO:29). The immunostimulatory oligonucleotide nucleic acid sequence of SEQ ID NO:29 differs from a previously reported immunostimulatory oligonucleotide (CpG 10103) 5′ TCGTCGTTTTTCGGTCGTTTT 3′ (SEQ ID NO:28) by the reversal of the 3′ most CG dinucleotide. The similarity in activity between these two immunostimulatory oligonucleotides is surprising because it has been previously reported that immunostimulatory activity of CpG oligonucleotides is dependent on the number of CpG motifs, the sequences flanking the CG dinucleotide, the location of the CpG motif(s) and the spacing between the CpG motifs (Ballas et al., 1996, J. Immunol.; Hartmann et al., 2000, J. Immunol.; Klinman et al., 2003, Clin. Exp. Immunol.). The removal of the 3′ most CG dinucleotide in immunostimulatory oligonucleotide CpG 24555 did not result in a negative impact on the ability of this immunostimulatory oligonucleotide to augment antigen-specific immune responses as would have been expected from previous disclosures. CpG 24555 demonstrated similar and in some cases enhanced immunostimulatory activity when compared with CpG 10103.

The immunostimulatory oligonucleotide can be double-stranded or single-stranded. Generally, double-stranded molecules are more stable in vivo, while single-stranded molecules have increased immune activity. Thus in some aspects of the disclosure it is preferred that the nucleic acid be single stranded and in other aspects it is preferred that the nucleic acid be double-stranded.

For any of the CpG sequences disclosed herein (e.g. CpG 24555, CpG 10103, and CpG 7909), any of the internucleotide linkages can be phosphorothioate or phosphodiester bonds.

The terms “nucleic acid” and “oligonucleotide” are used interchangeably herein to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). As used herein, the terms refer to oligoribonucleotides (i.e. a polynucleotide minus the phosphate) and any other organic base containing polymer. Nucleic acid molecules can be obtained from existing nucleic acid sources (e.g. genomic or cDNA), but are preferably synthetic (e.g. produced by nucleic acid synthesis).

In one embodiment, the immunostimulatory oligonucleotides can encompass various chemical modifications and substitutions, in comparison to natural RNA and DNA, involving a phosphodiester internucleoside bridge, a β-D-ribose (deoxyribose) unit and/or a natural nucleoside base (adenine, guanine, cytosine, thymine, uracil). Examples of chemical modifications are known to the skilled person and are described, for example in Uhlmann E. et al. (1990), Chem. Rev. 90:543; “Protocols for Oligonucleotides and Analogs”, Synthesis and Properties & Synthesis and Analytical Techniques, S. Agrawal, Ed., Humana Press, Totowa, USA 1993; Crooke, S. T. et al. (1996) Annu. Rev. Pharmacol. Toxicol. 36:107-129; and Hunziker J. et al., (1995), Mod. Synth. Methods 7:331-417. An oligonucleotide according to the disclosure may have one or more modifications, wherein each modification is located at a particular phosphodiester internucleoside bridge and/or at a particular β-D-(deoxy)ribose unit and/or at a particular natural nucleoside base position in comparison to an oligonucleotide of the same sequence which is composed of natural DNA or RNA.

For example, the oligonucleotides may comprise one or more modifications. Such modifications may be selected from: a) the replacement of a phosphodiester internucleoside bridge located at the 3′ and/or the 5′ end of a nucleoside by a modified internucleoside bridge, b) the replacement of phosphodiester bridge located at the 3′ and/or the 5′ end of a nucleoside by a dephospho bridge, c) the replacement of a sugar phosphate unit from the sugar phosphate backbone by another unit, d) the replacement of a β-D-ribose unit by a modified sugar unit, and e) the replacement of a natural nucleoside base.

Nucleic acids also include substituted purines and pyrimidines, such as C-5 propyne pyrimidine and 7-deaza-7-substituted purine modified bases (Wagner et al., 1996, Nat. Biotechnol. 14:840-4). Purines and pyrimidines include but are not limited to adenine, cytosine, guanine, thymidine, 5-methlycytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those skilled in the art.

A modified base is any base which is chemically distinct from the naturally occurring bases typically found in DNA and RNA, such as T, C, G, A, and U, but which shares basic chemical structures with these naturally occurring bases. The modified nucleoside base may be, for example, selected from hypoxanthine, uracil, dihydrouracil pseudouracil, 2-thiouracil, 4-thiouracil, 5-aminouracil, 5-(C1-C6)-alkyluracil, 5-(C2-C6)-alkenyluracil, 5-(C2-C6)-alkylnyluracil, 5-(hydroxymethyl)uracil, 5-chlorouracil, 5-fluorouracil, 5-bromouracil, 5-hydroxycytosine, 5-(C1-C6)-alkylcytosine, 5-(C2-C6)-alkenylcytosine, 5-(C2-C6)-alkylnylcytosine, 5-chlorocytosine, 5-fluorocytosine, 5-bromocytosine, N2-dimethylguanine, 2,4-dimaino-purine, 8-azapurine, a substituted 7-deazapurine, preferably 7-deaza-7-substituted and/or 7-deaza-8-substituted purine, 5-hydroxymethlycytosine, N4-alkylcytosine, e.g., N4-ethylcytosine, 5-hydroxydeoxycytidine, 5-hydroxymethyldeoxycytidine, N4-alkyldeoxycytidine, e.g. N4-ethyldeoxycytidine, 6-thiodeoxyguanosine, and deoxyribonucleosides of nitropyrrole, C5-propynylpyrimisine, and diaminopurine e.g., 2,6-diaminopurine, inosine, 5-methylcytosine, 2-aminopurine, 2-amino-6-chloropurine, hypoxanthine or other modifications of a natural nucleoside base. This list is meant to be exemplary and is not to be interpreted to be limiting.

In some aspects of the disclosure, the CpG dinucleotide of the immunostimulatory oligonucleotides described herein are preferably unmethylated. An unmethylated CpG motif is an unmethylated cytosine-guanine dinucleotide sequence (i.e. an unmethylated 5′ cytosine followed by 3′ guanosine and linked by a phosphate bond). In other aspects, the CpG motifs are methylated. A methylated CpG motif is a methylated cytosine-guanine dinucleotide sequence (i.e. a methylated 5′ cytosine followed by a 3′ guanosine and linked by a phosphate bond).

In some aspects of the disclosure, an immunostimulatory oligonucleotide can contain a modified cytosine. A modified cytosine is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the disclosure, the cytosine base is substituted by a universal base (e.g. 3-nitropyrrole, P-base), an aromatic ring system (e.g. fluorobenzene or difluorobenzene) or a hydrogen atom (dSpacer).

In some aspects of the disclosure, an immunostimulatory oligonucleotide can contain a modified guanine. A modified guanine is a naturally occurring or non-naturally occurring purine base analog of guanine which can replace this base without impairing the immunostimulatory activity of the oligonucleotide. Modified guanines include but are not limited to 7-deeazaguanine, 7-deaza-7-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine), 8-substituted guanine (e.g. 8-hydroxyguanine and 8-bromoguanine), and 6-thioguanine. In another embodiment of the disclosure, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

In certain aspects, the oligonucleotides may include modified internucleotide linkages. These modified linkages may be partially resistant to degradation (e.g. are stabilized). A “stabilized nucleic acid molecule” means a nucleic acid molecule that is relatively resistant to in vivo degradation (e.g. via an exo- or endo-nuclease). Stabilization can be a function of length or secondary structure. Nucleic acids that are tens to hundreds of kilobases long are relatively resistant to in vivo degradation. For shorter nucleic acids, secondary structure can stabilize and increase their effect. The formation of a stem loop structure can stabilize a nucleic acid molecule. For example, if the 3′ end of a nucleic acid has self-complementarity to an upstream region so that it can fold back and form a stem loop structure, then the nucleic acid can become stabilized and exhibit more activity.

For use in vivo, nucleic acids are preferably relatively resistant to degradation (e.g. via endo- and exo-nucleases). It has been demonstrated that modification of the nucleic acid backbone provides enhanced activity of nucleic acids when administered in vivo. Secondary structures, such as stem loops, can stabilize nucleic acids against degradation. Alternatively, nucleic acid stabilization can be accomplished via phosphate backbone modifications. A preferred stabilized nucleic acid has at least a partial phosphorothioate modified backbone. Phosphorothioates may be synthesized using automated techniques employing either phosphoramidate or H-phosphonate chemistries. Aryl- and alkyl-phosphonates can be made, e.g. as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A. (1990) Chem. Rev. 90:544; Goodchild, J. (1990) Bioconjugate Chem. 1:165). 2′-O-methyl nucleic acids with CpG motifs also cause immune activation, as do ethoxy-modified CpG nucleic acids. In fact, no backbone modifications have been found that completely abolish the CpG effect, although it is greatly reduced by replacing the C with a 5-methyl C. Constructs having phosphorothioate linkages provide maximal activity and protect the nucleic acid from degradation by intracellular exo- and endo-nucleases. Other modified nucleic acids include phosphodiester modified nucleic acids, combinations of phosphodiester and phosphorothioate nucleic acid, methylphosphonate, methylphosphorothioate, phosphorordithioate, p-ethoxy, and combinations thereof. Each of these combinations and their particular effects on immune cells is discussed in more detail with respect to CpG nucleic acids in PCT Publication Nos. WO 96/02555 and WO 98/18810 and in U.S. Pat. Nos. 6,194,388 and 6,239,116. It is believed that these modified nucleic acids may show more stimulatory activity due to enhanced nuclease resistance, increased cellular uptake, increased protein binding, and/or altered intracellular localization.

For administration in vivo, nucleic acids may be associated with a molecule that results in higher affinity binding to target cell (e.g. dendritic cell, B-cell, monocytic cell and natural killer (NK) cell) surfaces and/or increased cellular uptake by target cells to form a “nucleic acid delivery complex”. Nucleic acids can be ionically, or covalently associated with appropriate molecules using techniques which are well known in the art. A variety of coupling or crosslinking agents can be used, e.g. protein A, carbodiimide, and N-succinimidyl-3-(2-pyridyldithio) propionate (SPDP). Nucleic acids can alternatively be encapsulated in liposomes or virosomes using well-known techniques.

Other stabilized nucleic acids include, but are not limited to, nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation. In some embodiments, an immunostimulatory oligonucleotide of the disclosure may include at least one lipophilic substituted nucleotide analog and/or a pyrimidine-purine dinucleotide.

The oligonucleotides may have one or two accessible 5′ ends. It is possible to create modified oligonucleotides having two such 5′ ends, for instance, by attaching two oligonucleotides through a 3′-3′ linkage to generate an oligonucleotide having one or two accessible 5′ ends. The 3′-3′-linkage may be a phosphodiester, phosphorothioate or any other modified internucleoside bridge. Methods for accomplishing such linkages are known in the art. For instance, such linkages have been described in Seliger, H. et al., Oligonucleotide analogs with terminal 3′-3′- and 5′-5′-internucleotidic linkages as antisense inhibitors of viral gene expression, Nucleosides & Nucleotides (1991), 10 (1-3), 469-77 and Jiang, et al., Pseudo-cyclic oligonucleotides: in vitro and in vivo properties, Bioorganic & Medicinal Chemistry (1999), 7 (12), 2727-2735.

Additionally, 3′-3′-linked oligonucleotides where the linkage between the 3′-terminal nucleosides is not a phosphodiester, phosphorothioate or other modified bridge, can be prepared using an additional spacer, such as tri- or tetra-ethyleneglycol phosphate moiety (Durand, M. et al., Triple-helix formation by an oligonucleotide containing one (dA)12 and two (dT)12 sequences bridged by two hexaethylene glycol chains, Biochemistry (1992), 31 (38), 9197-204, U.S. Pat. Nos. 5,658,738 and 5,668,265). Alternatively, the non-nucleotidic linker may be derived from ethanediol, propanediol, or from an abasic deoxyribose (dSpacer) unit (Fontanel, Marie Laurence et al., Nucleic Acids Research (1994), 22 (11), 2022-7) using standard phosphoramidite chemistry. The non-nucleotidic linkers can be incorporated once or multiple times, or combined with each other allowing for any desirable distance between the 3′-ends of the two oligonucleotides to be linked.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR₁R₂-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)—O-alkyl]ester, (C₁-C₈)alkylphosphonate and/or (C₆-C₁₂)arylphosphonate bridges, (C₇-C₁₂)-α-hydroxymethyl-aryl (e.g. disclosed in PCT Publication No. WO 95/01363), wherein (C₆-C₁₂)aryl, (C₆-C₂₀)aryl and (C₆-C₁₄)aryl are optionally substituted by halogen, alkyl, alkoxy, nitro, cyano, and where R₁ and R₂ are, independently of each other, hydrogen, (C₁-C₁₈)-alkyl, (C₆-C₂₀)-aryl, (C₆-C₁₄)-aryl, (C₁-C₈)-alkyl, preferably hydrogen, (C₁-C₈)-alkyl, preferably (C₁-C₄)-alkyl and/or methoxyethyl, or R₁ and R₂ form, together with the nitrogen atom carrying them, a 5 to 6-membered heterocyclic ring which can additionally contain a further heteroatom selected from the group O, S and N.

The replacement of a phosphodiester bridge located at the 3′ and/or the 5′ end of a nucleoside by a dephospho bridge (dephospho bridges are described, for example, in Uhlmann E. and Peyman A. in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

The immunostimulatory oligonucleotides of the disclosure may optionally have chimeric backbones. A chimeric backbone is one that comprises more than one type of linkage. In one embodiment, the chimeric backbone can be represented by the formula: 5′ Y1N1ZN2Y2 3′. Y1 and Y2 are nucleic acid molecules having between 1 and 10 nucleotides. Y1 and Y2 each include at least one modified internucleotide linkage. Since at least 2 nucleotides of the chimeric oligonucleotides include backbone modifications these nucleic acids are an example of one type of “stabilized immunostimulatory nucleic acids”.

With respect to the chimeric oligonucleotides, Y1 and Y2 are considered independent of one another. This means that each of Y1 and Y2 may or may not have different sequences and different backbone linkages from one another in the same molecule. In some embodiments, Y1 and/or Y2 have between 3 and 8 nucleotides. N1 and N2 are nucleic acid molecules having between 0 and 5 nucleotides as long as N1ZN2 has at least 6 nucleotides in total. The nucleotides of N1ZN2 have a phosphodiester backbone and do not include nucleic acids having a modified backbone. Z is an immunostimulatory nucleic acid motif, preferably selected from those recited herein.

The center nucleotides (N1ZN2) of the formula Y1N1ZN2Y2 have phosphodiester internucleotide linkages and Y1 and Y2 have at least one, but may have more than one or even may have all modified internucleotide linkages. In preferred embodiments, Y1 and/or Y2 have at least two or between two and five modified internucleotide linkages or Y1 has five modified internucleotide linkages and Y2 has two modified internucleotide linkages. The modified internucleotide linkage, in some embodiments, is a phosphorothioate modified linkage, a phosphorodithioate linkage or a p-ethoxy modified linkage.

The nucleic acids also include nucleic acids having backbone sugars which are covalently attached to low molecular weight organic groups other than a hydroxyl group at the 2′ position and other than a phosphate group at the 5′ position. Thus, modified nucleic acids may include a 2′-O-alkylated ribose group. In addition, modified nucleic acids may include sugars such as arabinose or 2′-fluoroarabinsoe instead of ribose. Thus, the nucleic acids may be heterogeneous in backbone composition thereby containing any possible combination of polymer units linked together such as peptide-nucleic acids (which have amino acid backbone with nucleic acid bases). In some embodiments, the nucleic acids are homogeneous in backbone composition.

A sugar phosphate unit (i.e. a β-D-ribose and phosphodiester internucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E. P. et al. (1989) Nucleic Acid Res. 17:6129-41), that is, e.g., the replacement by a morpholino-derivative; or to build up a polyamide nucleic acid (“PNA”; as described for example, in Nielsen P. E. et al. (1994) Bioconjug. Chem. 5:3-7), e.g., the replacement by a PNA backbone unit, e.g., by 2-aminoethylglycine. The oligonucleotide may have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker may be branched or unbranched, substituted or unsubstituted, and chirally pure or a racemic mixture.

A β-ribose unit or a β-D-2′ deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆) alkyl-ribose is 2′-O-methylribose, 2′-O—(C₁-C₆)alkenyl-ribose, 2′-[O—(C₁-C₆)alkyl-O—(C₁-C₆)alkyl]-ribose, 2′—NH2-2′-deoxyribose, β-D-xylo-furanose, α-arabinofuranose, 2,4-dideoxy-β-D-erythro-hexo-pyranose, and carbocyclic (described, for example, in Froehler J. (1992) Am. Chem. Soc. 114:8320) and/or open-chain sugar analogs (described, for example, in Vandendriessche et al. (1993) Tetrahedron 49:7223) and/or bicyclosugar analogs (described, for example, in Tarkov M. et al. (1993) Helv. Chim. Acta. 76:481.

In some embodiments, the sugar is 2′-O-methylribose, particularly for one or both nucleotides linked by a phosphodiester or phosphodiester-like internucleoside linkage.

The oligonucleotides of the disclosure can be synthesized de novo using any of a number of procedures well known in the art. For example, the b-cyanoethyl phosphoramidite method (Beaucage, S. L., and Caruthers, M. H., (1981) Tet. Let. 22:1589); nucleoside H-phosphonate method (Garegg et al., (1986) Tet. Let. 27:4051-4054; Froehler et al., (1986) Nucl. Acid Res. 14:5399-5407; Garegg et al., (1986) 27:4055-4058; Gaffney et al., (1988) Tet. Let. 29:2619-2622). These chemistries can be performed by a variety of automated nucleic acid synthesizers available in the market. These oligonucleotides are referred to as synthetic oligonucleotides. Alternatively, T-rich and/or TG dinucleotides can be produced on a large scale in plasmids, (see Sambrook T. et al., “Molecular Cloning: A Laboratory Manual”, Cold Spring Harbor laboratory Press, New York, 1989) and separated into smaller pieces or administered whole. Nucleic acids can be prepared from existing nucleic acid sequences (e.g. genomic or cDNA) using known techniques, such as those employing restriction enzymes, exonucleases or endonucleases.

Modified backbones such as phosphorothioates may be synthesized using automated techniques employing either phosphoramidate or H-phosphonate chemistries. Aryl- and alkyl-phoshonates can be made, e.g. as described in U.S. Pat. No. 4,469,863, and alkylphosphotriesters (in which the charged oxygen moiety is alkylated as described in U.S. Pat. No. 5,023,243) can be prepared by automated solid phase synthesis using commercially available reagents. Methods for making other DNA backbone modifications and substitutions have been described (e.g. Uhlmann, E. and Peyman, A., Chem. Rev. 90:544, 1990; Goodchild, J., Bioconjugate Chem. 1:165, 1990).

Nucleic acids prepared in this manner are referred to as isolated nucleic acids. An “isolated nucleic acid” generally refers to a nucleic acid which is separated from components with which it is separated from a cell, from a nucleus, from mitochondria or from chromatin and from any other components that may be considered as contaminants.

In some embodiments, CpG-containing oligonucleotides might be simply mixed with immunogenic carriers according to methods known to those skilled in the art (see for example PCT Publication No. WO 03/024480). In other embodiments of the disclosure, CpG-containing oligonucleotides might be enclosed within VLPs (see e.g. PCT Publication No. WO 03/024481).

Examples of adjuvants in the context of the present disclosure include alum; CpG-containing oligonucleotides, such as CpG 7909, CpG 10103, and CpG 24555; and saponin-based adjuvants, such as Iscomatrix, which could be used alone or in combination.

The disclosure therefore provides an immunogenic composition comprising an antigenic tau peptide, preferably comprising an amino acid sequence selected from the group consisting of SEQ ID NOs: 1 to 26, 31 to 76, and 105-122, and at least one adjuvant. Said antigenic tau peptide is preferably linked to an immunogenic carrier, preferably a VLP, more preferably an HBsAg, HBcAg or Qbeta VLP. In one embodiment, said adjuvant is a saponin-based adjuvant, preferably Iscomatrix. In another embodiment, said adjuvant is Alum. In still another embodiment, said adjuvant is a CpG-containing oligonucleotide. Preferably said adjuvant is CpG 7909 or CpG 10103. More preferably said adjuvant is CpG 24555.

In still another embodiment, said at least one adjuvant comprises two adjuvants, preferably selected from the group consisting of Alum, sapoinin-based adjuvants, and CpG-containing oligonucleotides. In a preferred embodiment, said adjuvants are Alum and a CpG-containing oligonucleotide, preferably CpG 7909, preferably CpG 10103, more preferably CpG 24555. In another preferred embodiment, said adjuvants are a saponin-based adjuvant, preferably Iscomatrix, and a CpG-containing oligonucleotide, preferably CpG 7909, preferably CpG 10103, more preferably CpG 24555. In another preferred embodiment, said adjuvants are Alum and a saponin-based adjuvant, preferably Iscomatrix.

In still another embodiment, said at least one adjuvant comprises three adjuvants, preferably selected from the group consisting of Alum, a saponin-based adjuvant, preferably Iscomatrix, and CpG-containing oligonucleotides, such as CpG 7909, CpG 10103, and CpG 24555.

### Formulations

The present disclosure also provides pharmaceutical compositions comprising an antigenic tau peptide of the disclosure or an immunogenic composition thereof, in a formulation in association with one or more pharmaceutically acceptable excipient(s). The term ‘excipient’ is used herein to describe any ingredient other than the active ingredient, i.e. the antigenic tau peptide of the disclosure eventually coupled to an immunogenic carrier and optionally combined with one or more adjuvants. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the active ingredient.

Pharmaceutical compositions of the present disclosure and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP conditions.

A pharmaceutical composition of the disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The pharmaceutical compositions of the disclosure are typically suitable for parenteral administration. As used herein, “parenteral administration” of a pharmaceutical composition includes any route of administration characterised by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. Preferred embodiments include the intravenous, subcutaneous, intradermal, and intramuscular routes.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the antigenic tau peptide, preferably coupled to a an immunogenic carrier, eventually in combination with one or more adjuvants, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An exemplary, non-limiting pharmaceutical composition of the disclosure is a formulation as a sterile aqueous solution having a pH that ranges from about 5.0 to about 6.5 and comprising from about 1 mg/mL to about 200 mg/mL of a peptide of the disclosure, from about 1 millimolar to about 100 millimolar of histidine buffer, from about 0.01 mg/mL to about 10 mg/mL of polysorbate 80, from about 100 millimolar to about 400 millimolar of trehalose, and from about 0.01 millimolar to about 1.0 millimolar of disodium EDTA dihydrate.

The antigenic tau peptides of the disclosure can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurised container, pump, spray, atomiser (preferably an atomiser using electrohydrodynamics to produce a fine mist), or nebuliser, with or without the use of a suitable propellant, or as nasal drops.

The pressurized container, pump, spray, atomizer, or nebuliser generally contains a solution or suspension of a composition of the disclosure comprising, for example, a suitable agent for dispersing, solubilizing, or extending release of the active, and a propellant(s) as solvent.

Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules, blisters and cartridges for use in an inhaler or insufflator may be formulated to contain a powder mix of the compound of the disclosure, a suitable powder base and a performance modifier.

A suitable solution formulation for use in an atomizer using electrohydrodynamics to produce a fine mist may contain a suitable dose of the antigenic tau peptide of the disclosure per actuation and the actuation volume may for example vary from 1 μL to 100 μL.

Suitable flavors, such as menthol and levomenthol, or sweeteners, such as saccharin or saccharin sodium, may be added to those formulations of the disclosure intended for inhaled/intranasal administration.

Formulations for inhaled/intranasal administration may be formulated to be immediate and/or modified release. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the disclosure are typically arranged to administer a metered dose or “puff” of a composition of the present disclosure. The overall daily dose will typically be administered in a single dose or, more usually, as divided doses throughout the day.

A pharmaceutical composition comprising an antigenic tau peptide may also be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

### Dosages

The compositions of the disclosure can be used to treat, alleviate or prevent tau-related disorders or symptoms in a subject at risk or suffering from such disorder or symptom by stimulating an immune response in said subject by immunotherapy. Immunotherapy can comprise an initial immunization followed by additional, e.g. one, two, three, or more boosters.

An “immunologically effective amount” of an antigenic tau peptide of the disclosure, or composition thereof, is an amount that is delivered to a mammalian subject, either in a single dose or as part of a series, which is effective for inducing an immune response against pathological forms of tau in said subject. This amount varies depending upon the health and physical condition of the individual to be treated, the taxonomic group of individual to be treated, the capacity of the individual's immune system to elicit humoral and/or cellular immune responses, the formulation of the vaccine, and other relevant factors. It is expected that the amount will fall in a relatively broad range that can be determined through appropriate trials.

A “pharmaceutically effective dose” or “therapeutically effective dose” is that dose required to treat or prevent, or alleviate one or more tau-related disorders or symptoms in a subject. The pharmaceutically effective dose can depend on the specific compound to administer, the severity of the symptoms, the susceptibility of the subject to side effects, the type of disease, the composition used, the route of administration, the type of mammal being treated, the physical characteristics of the specific mammal under consideration such as health and physical condition, concurrent medication, the capacity of the individual's immune system, the degree of protection desired, and other factors that those skilled in the medical arts will recognize. For prophylaxis purposes, the amount of peptide in each dose is selected as an amount which induces an immunoprotective response without significant adverse side effects in typical vaccines. Following an initial vaccination, subjects may receive one or several booster immunizations adequately spaced.

It is understood that the specific dose level for any particular patient depends upon a variety of factors including the activity of the specific compound employed, the age, body weight, general health, gender, diet, time of administration, route of administration, rate of excretion, drug combination and the severity of the particular disease undergoing therapy.

For example, antigenic tau peptides of the disclosure, coupled to an immunogenic carrier, can be administered to a subject at a dose of about 0.1 μg to about 200 mg each, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 10 mg, from about 10 mg to about 50 mg, or from about 50 mg to about 200 mg, with optional boosters given at, for example, 1 week, 2 weeks, 3 weeks, 4 weeks, 2 months, 3 months and/or a year later. In some embodiments, the amount of antigenic tau peptide per dose is determined on a per body weight basis. For example, in some embodiments, an antigenic peptide is administered in an amount of from about 0.5 mg/kg to about 100 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg per dose, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg, or more than about 100 mg/kg.

In some embodiments, a single dose of an antigenic tau peptide according to the disclosure is administered. In other embodiments, multiple doses of an antigenic tau peptide according to the disclosure are administered. The frequency of administration can vary depending on any of a variety of factors, e.g., severity of the symptoms, degree of immunoprotection desired, whether the composition is used for prophylactic or curative purposes, etc. For example, in some embodiments, an antigenic tau peptide according to the disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). When the composition of the disclosure is used for prophylaxis purposes, it will be generally administered for both priming and boosting doses. It is expected that the boosting doses will be adequately spaced, or preferably given yearly or at such times where the levels of circulating antibody fall below a desired level. Boosting doses may consist of the antigenic tau peptide in the absence of the original immunogenic carrier molecule. Such booster constructs may comprise an alternative immunogenic carrier or may be in the absence of any carrier. Such booster compositions may be formulated either with or without adjuvant.

The duration of administration of an antigenic tau peptide according to the disclosure, e.g., the period of time over which an antigenic tau peptide is administered, can vary, depending on any of a variety of factors, e.g., patient response, etc. For example, an antigenic tau peptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Uses and Methods of Treatment

A variety of treatment methods are also contemplated by the present disclosure, which methods comprise administering an antigenic tau peptide according to the disclosure. Treatment methods include methods of inducing an immune response in an individual to self-tau in its pathological form(s), and methods of preventing, alleviating or treating a tau-related disorder or symptom in an individual.

In one aspect, the present disclosure provides a method for treating, preventing or alleviating a tau-related disorder or symptom in a subject, comprising administering a therapeutically effective amount of an antigenic tau peptide of the disclosure, or immunogenic or pharmaceutical composition thereof, to said subject.

In another aspect, the present disclosure provides a method for inducing an immune response against self-tau in its pathological form(s) in a subject, comprising administering a therapeutically or immunogenically effective amount of an antigenic tau peptide of the disclosure, or immunogenic or pharmaceutical composition thereof, to said subject.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Said subject is preferably human, and may be either male or female, of any age.

Other aspects of the disclosure relate to an antigenic tau peptide according to the disclosure, or of an immunogenic composition or a pharmaceutical composition thereof, for use as a medicament, preferably in the treatment of tau-related disorders.

In yet another aspect, the present disclosure provides the use of an antigenic tau peptide of the disclosure or of an immunogenic composition or a pharmaceutical composition thereof, in the manufacture of a medicament, preferably for treating a tau-related disorder.

The present disclosure is further illustrated by the following examples which should not be construed as further limiting. The contents of all figures and all references, patents, and published patent applications cited throughout this disclosure are expressly incorporated herein by reference in their entirety.

### EXAMPLES

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. As used in the Examples below, the following abbreviations have the following meanings, and unless indicated otherwise, are readily available from commercial suppliers: DMF: dimethylformamide; TFA: trifluoroacetic acid; TIPS: triisopropylsilyl trifluoromethanesulfonate; TCEP: tris(2-carboxyethyl)phosphine; mcKLH: maricultured keyhole limpet hemocyanin; HBTU: O-benzotriazole-N,N,N′N′-tetramethyl-uronium-hexafluoro-phosphate; EDTA: ethylene-diamine-tetraacetic acid; DMSO: dimethyl sulfoxide.

### Example 1

### Qbeta Plasmid Construction

Native Qbeta coat protein: The coding sequence corresponding to the coat protein of Qbeta, nucleotides 1304 to 1705 from GenBank Accession # AY099114, was synthesized by DNA 2.0 (DNA 2.0, Menlo Park, Calif.). A 5′ modification (CCatgg) to introduce an NcoI site and 3′ modifications to introduce two stop codons and an XhoI site (gtaTTAATGACTCGAG—SEQ ID NO: 78) were included.

Codon Optimized Qbeta coat protein: The Qbeta coat protein coding sequence was also optimized for expression using Gene Designer (Villalobos et al., BMC Bioinformatics 7:285 (2006)). The identical 5′ and 3′ modifications were incorporated into the codon optimized Qbeta coat protein.

Both native and codon optimized Qbeta coat protein sequences were introduced into a pET28 expression vector using conventional DNA subcloning methods including restriction digestion and ligation reactions.

### Example 2

### Preparation of Synthetic Tau Peptides

Tau peptides (referred to as A-1 to A-11; B-1 to B-6; C-1 to C-5; D-1; E-1, and F1; along with phosphorylated versions of these peptides—indicated as A-1P, A-2P, A-3P, etc.) as set forth as SEQ ID NOs. 31-76, 105-107, and shown in Table 5 below with their corresponding names as used throughout the following examples, were prepared as follows. Synthesis of phosphorylated or non-phosphorylated tau peptides containing a linker sequence (CGG or GGC) were performed using solid phase synthesis technology on a Symphony peptide synthesizer (Protein Technologies, Inc). The mono-protected amino acid Fmoc-Ser[PO(O-Bzl)OH]—OH,Fmoc-Thr[PO(O-Bzl)OH]—OH, and Fmoc-Tyr[PO(O-Bzl)OH]—OH (EMD Chemicals, Inc) were used for incorporating phosphoserine, phosphothreonine, and phosphotyrosine into the phosphorylated versions of the sequences. The reaction was initiated by mixing the NovaSyn TGA resin (EMD Chemicals, Inc) containing the first amino acid with 6.25 fold excess of Fmoc-protected second amino acid (1 mmol) which was activated with 1 mmol of HBTU for 1 hour. The coupling reaction was repeated once for each amino acid. Removal of the Fmoc group was achieved in 20% piperidine in DMF for 2×5 minutes. The synthesized peptide was released from the resin by incubating the resin with 5 mL of TFA solution containing 2.5% TIPS and 2.5% Thioanisole for 3 hours at room temperature. The crude peptide was recovered following filtration, diethyl ether-mediated precipitation, and vacuum-drying. Purification of the peptide was performed in a reverse phase HPLC (Waters 2525 Binary Gradient Module) with a BEH 130 Preparative C18 column. The mobile phase consisted of 0.1% TFA in water as the buffer A and 0.1% TFA in acetonitrile as the buffer B. The collected fractions containing the peptide were combined and lyophilized under vacuum. Approximately 20 mg of the peptide was purified from a typical injection of 100 mg of the crude peptide with a purity of more than 95%. All purified peptides were verified with LC-MS.

Similarly, additional tau peptides (SEQ IDs: 108-122) are synthesized and purified.

### Example 3

### Qbeta-VLP: Expression, Purification, and Conjugation with Tau Peptides

Expression of Qbeta in E. coli: The plasmid pET28 containing Qbeta cDNA was transformed into E. coli BL21(DE3) competent cells. A single colony was inoculated in 5 mL of 2×YT medium containing 50 μg/mL kanamycin at 37° C. overnight. The overnight inoculum was diluted to 500 mL of TB medium containing 50 μg/mL kanamycin, grew to 0.8 OD600 at 250 rpm at 37° C., and induced with 0.4 mM IPTG (isopropyl β-D-1-thiogalactopyranoside) overnight. The cells were harvested by centrifuging at 2500 RCF for 15 minutes. The cell pellets were stored at −80° C.

Purification of Qbeta VLP from E. coli: All purification steps were performed at 4° C. The cell pellet expressing Qbeta was resuspended in a lysis buffer containing 25 mM Tris pH 8.0, 150 mM NaCl, 5 mM EDTA, 0.1% Triton-100 supplemented with protease inhibitor cocktails (Roche). The resuspension solution was passed through a microfluidizer (Microfluidics Corp.), followed by ultracentrifugation. Proteins were precipitated by adding ammonium sulfate to 50% saturation followed by centrifugation at 15,000 RCF for 30 minutes. The pellet was resuspended and dialyzed in the buffer containing 25 mM Hepes pH 7.5, 100 mM NaCl, 1 mM EDTA at 4° C. overnight. The dialyzed solution was centrifuged and then loaded into a Capto Q column (GE) equilibrated in 25 mM HEPES pH 7.5, 100 mM NaCl, 1 mM EDTA. The column was washed and run with a gradient from 100 mM NaCl to 1 M NaCl in the buffer containing 25 mM HEPES pH 7.5, 1 mM EDTA. Qbeta protein was identified using SDS-PAGE. Fractions containing Qbeta were dialyzed in 10 mM potassium phosphate, pH 7.4, 150 mM KCl overnight, and loaded into a hydroxyapatite column (Type II, Bio-Rad Inc.). The column was washed and eluted with a gradient from 100% of buffer containing 10 mM potassium phosphate pH 7.5, 150 mM KCl to 100% of buffer containing 500 mM potassium phosphate, pH 7.5, 0.5M KCl. The fractions containing Qbeta were pooled, dialyzed, and loaded into a phenyl column equilibrated in 25 mM Tris-Cl, pH 8.0, 150 mM NaCl, 0.7 M (NH4)₂SO₄. The protein was eluted with a gradient from 100% of a buffer containing 25 mM Tris-Cl, pH 8.0, 150 mM NaCl, 0.7 M (NH₄)₂SO₄ to 100% of a buffer containing 25 mM Tris-Cl, pH 8.0, 50 mM NaCl. Fractions containing pure Qbeta were pooled and dialyzed in PBS at 4° C. overnight. The protein concentration was determined by Bradford assay.

Coupling of tau peptides to Qbeta VLP: The coupling of tau peptides to the Qbeta-VLP was mediated through a bifunctional cross-linker SMPH (Succinimidyl-6-[β-maleimidopropionamido]hexanoate) (Thermo Scientific) (Freer et al., Virology 322 (2):360-369 (2004)). The peptide was dissolved in PBS (Invitrogen), pH 7.0 containing 5 mM EDTA at 10 mg/mL, and reduced by incubating with the Immobilized TCEP disulfide reducing gel in an equal volume at room temperature for 1 hour. The peptide solution was recovered by centrifuging at 1000 times g for 2 minutes. The Qbeta-VLP protein at 2 mg/mL in PBS (Invitrogen) was activated by incubating with 7 mM SMPH in DMSO at room temperature for 1 hour. The derivatized VLP was desalted by passing through a Zeba Desalt Spin column (Thermo Scientific) at 1000 times g for 2 minutes. The activated VLP solution was mixed with 10-fold molar excess of reduced peptides at room temperature for 2 to 3 hours. The reaction mixture was concentrated and dialyzed either in PBS or 25 mM Histidine pH 7.4 containing 50 mM NaCl at 4° C. overnight. The protein concentration was determined with the Coomassie Plus protein assay from Thermo Scientific.

### Example 4

### Preparation of Peptide-KLH Conjugate

The tau peptide A-1P with a CGG linker (SEQ ID NO:31) was conjugated to mcKLH (Thermo Scientific, Cat. No. 77605) to assess its immunogenicity in mice. The conjugation was mediated through a bifunctional cross-linker SMPH (Succinimidyl-6-[β-maleimidopropionamido]hexanoate)(Thermo Scientific). The A-1P peptide at 10 mg/mL in PBS, pH 7.0 containing 5 mM EDTA was first treated with the Immobilized TCEP disulfide reducing gel in an equal volume by agitating at room temperature for 1 hour. The peptide solution was recovered by centrifuging at 1000×g for 2 minutes. The activation of KLH was carried out by incubating KLH at 10 mg/mL in PBS with 200 μL of 100 mM SMPH in DMSO for 1 hour at room temperature. The reaction mixture was allowed to pass through a Zeba Desalt Spin column (Thermo Scientific). The collected derivatized KLH was then mixed with the reduced A-1P for 2 hours at room temperature. The reaction mixture was dialyzed in PBS containing 0.6 M NaCl at 4° C. overnight. The protein concentration was determined with the Coomassie Plus protein assay from Thermo Scientific.

### Example 5

### Peptide Immunization Study for Immunogenicity and B Cell Memory

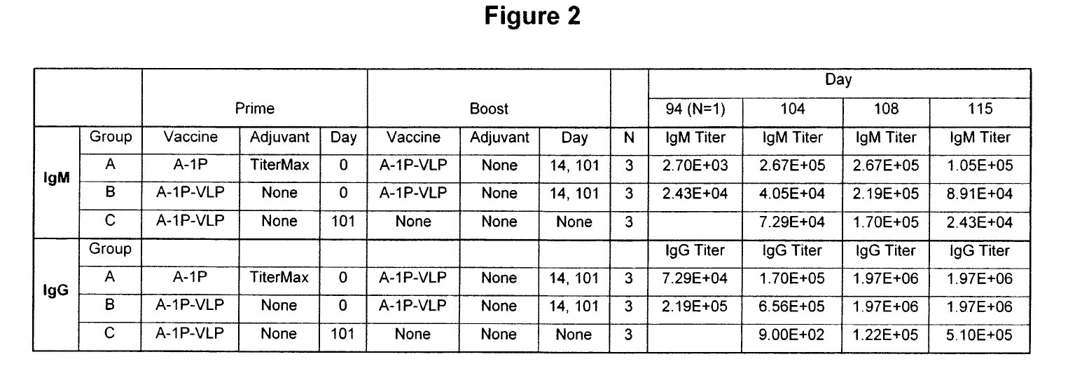

An experiment was performed to determine if select peptides shown in Table 5 were immunogenic and to determine if immunologic memory developed. Groups of 3 Balb/c mice were primed with peptide or peptide conjugated to Qbeta VLP on Day 0 and boosted on days 14 and 101 while some mice were only primed on day 101 as shown in FIGS. 1A, 1B, and 2. Sera were collected on days 28, 101, 104, 108 and 115. Sera from select mice were collected on day 94. Antibody responses from immunized animals were investigated using the antigen specific titer determination assays (as described in Example 13).

The antigen specific IgG titer results that show the peptides are immunogenic using serum samples from day 28 are summarized in FIG. 1B. This study showed that peptides A-1, A-1P, B-1P and C-1P are immunogenic when immunized using TiterMax Gold (Alexis Biochemicals) as an adjuvant. A prime with the A-1P peptide and TiterMax Gold or with A-1P conjugated to Qbeta-VLP, followed by a Day 14 boost with A-1P-Qbeta-VLP produced antibody titers greater than the A-1P TiterMax prime boost group. A prime and day 14 boost with A-1P conjugated to KLH (prepared as described in Example 4) as an adjuvant also produced antibody titers greater than the A-1P TiterMax prime boost group.

The selectivity of the antibodies elicited to the phosphorylated (A-1P, B-1P, D-1P, C-1P) or non-phosphorylated peptide (A-1) used for immunization were also examined. This was done by comparing the antibody titer to both the phosphorylated and non-phosphorylated versions for each peptide used for immunization (see FIG. 1B). The ratio of specific versus non specific titer was calculated. In this experiment, the antibody response against A-1 (Group 1) was selective (<0.1 fold) for the phosphorylation state of the peptides the animals were immunized with while the antibodies against C-1P (Group 5) are likely to be selective (>7 C-1P/C-1 titer ratio). Group 2 (A-1P) was not selective.

The results showing an A-1P B cell memory recall response are shown in FIG. 2. Group A (A-1P with TiterMax prime, boost with A-1P-Qbeta-VLP) and Group B (prime and boost with A-1P-Qbeta-VLP) were compared with Group C, which was primed on day 101 with A-1P conjugated to Qbeta-VLP. All three groups had an IgM response. IgG was detected at day 104 in both groups boosted on day 101 but not until day 7 for the group primed on day 101. The day 104 titers were greater than the day 94 titers. The IgG titers on day 7 and 14 were also greater than the day 101 prime group (Group C). The Group A and B IgG titers on days 108 and 115 were the same while the Group C IgG titer did not peak until day 115. These data are indicative of a long term antibody response and B cell memory recall.

### Example 6

### Peptide Prime and Peptide-VLP Boost Immunization Study for Immunogenicity

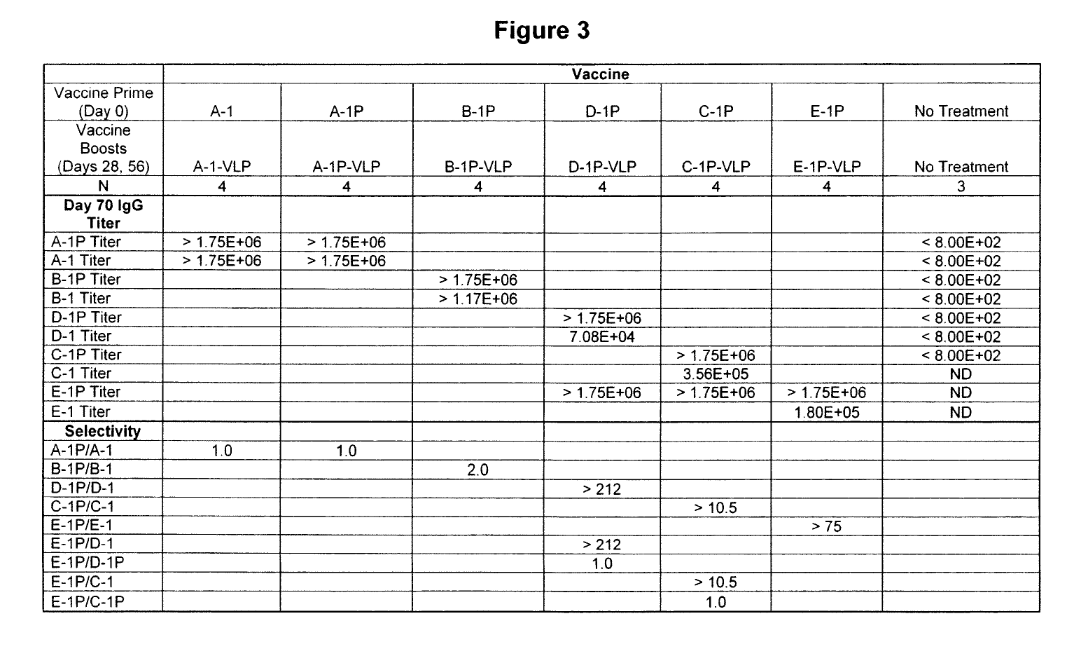

An experiment was performed to determine if select peptides from Table 5 were immunogenic when immunized as a peptide prime adjuvanted with alum (Al(OH)₃; Alhydrogel 2% ‘85’, Brenntag Biosector) followed by a boost with a peptide conjugated to the Qbeta-VLP. Groups of 4 Balb/c mice were primed on day 0 and boosted on days 28 and 56 as shown in FIG. 3. Sera were collected on day 70. Antibody responses from immunized animals were investigated using the antigen specific titer determination assay (as described in Example 13).

The results are summarized in FIG. 3. In Groups 1-6, IgG antibodies against the peptide used for immunization were detected at the maximum dilution tested (1:1,749,600), indicating robust antibody response to the immunized peptide antigen. No antibodies were detected in the untreated group (Group 7). The antibodies generated by immunization using peptides D-1P and C-1P recognized peptide E-1P. Peptides D-1P and C-1P are wholly contained within E-1P.

The selectivities of the antibodies elicited by the phosphorylated (A-1P, B-1P, D-1P, C-1P, E-1P) or non-phosphorylated peptide (A-1) used for immunization were examined. This was done by determining the antibody titer to the non-phosphorylated versions of the phosphorylated peptides and the phosphorylated versions of the non-phosphorylated peptides (see FIG. 3). The ratio of specific versus non specific titer was calculated. In this experiment, the antibodies against D-1P (Group 4), C-1P (Group 5) and E-1P (Group 6) were selective (>10 titer ratio) for the phosphorylation state of the peptides with which the animals were immunized.

### Example 7

### Peptide-VLP Immunization Study for Immunogenicity

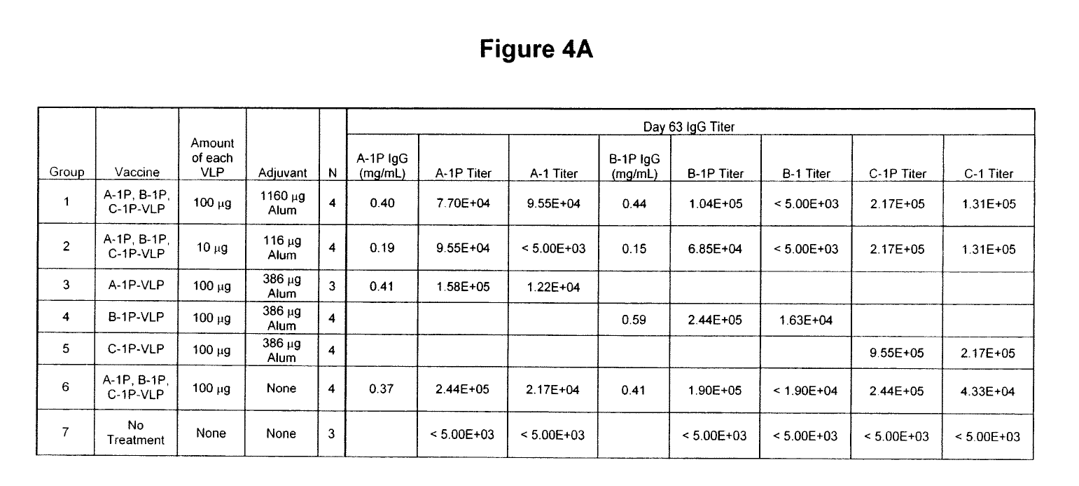

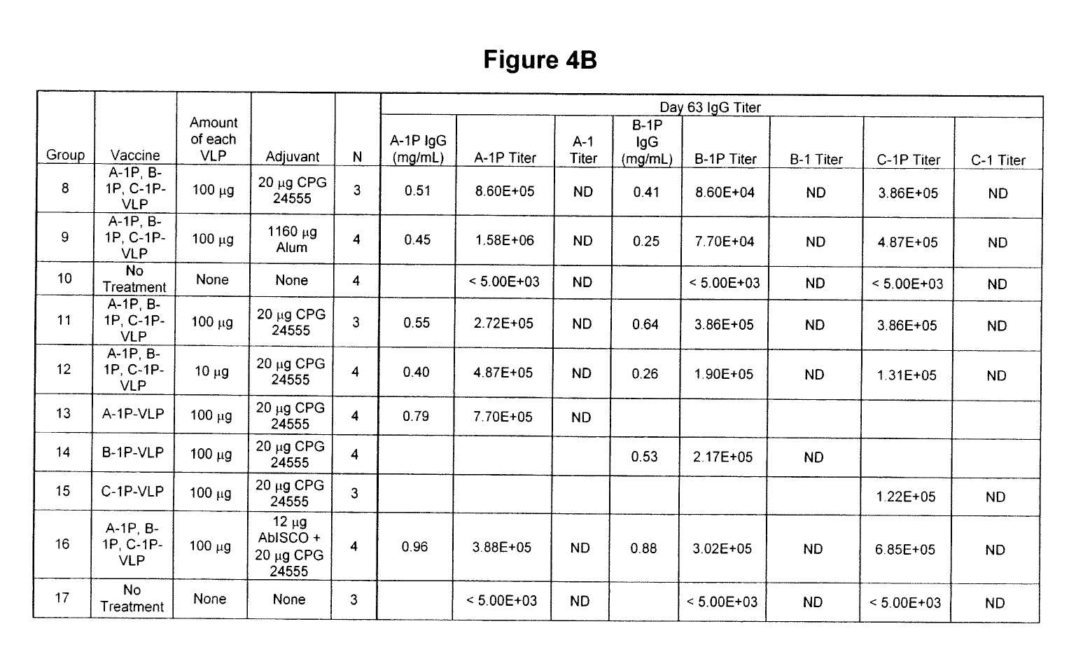

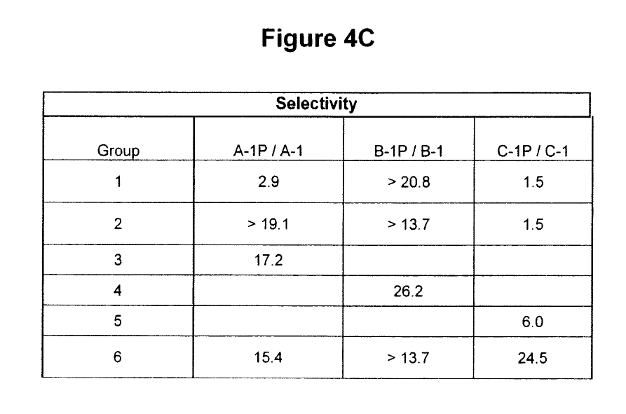

An experiment was performed to determine if select peptides and combinations of peptides from Table 5 were immunogenic when immunized as Qbeta-VLP conjugates with various adjuvants. As shown in FIG. 4, groups of 4 TG4510+/+ (transgenic double positive, see Ramsden et al, J. Neuroscience 25(46):10637 (2005)) or TG4510−/− (wild type litter mate control) mice were primed on day 0 and boosted on day 56 and either day 28 or 29. Sera were collected on day 63. Antibody responses from immunized animals were investigated using the antigen specific IgG titer determination assay as described in Example 13.

The day 63 sample results are summarized in FIG. 4. In every group, antibodies (IgG) against the peptide or combination of peptides used for immunization were detected at average titers ranging from 7.7 E+04 to 1.58 E+06. Immunizing three peptide-Qbeta-VLP conjugates in combinations of 100 μg or 10 μg each elicited similar titers to immunizing 100 μg of a peptide-Qbeta-VLP conjugate alone. The A-1P, B-1P and C-1P titers of combination dosing Groups 1 and 2, are 1.7 to 4.4 fold those of the relevant single dosing groups (Groups 3, 4 and 5). The A-1P, B-1P and C-1P titers of combination dosing Groups 11 and 12, are 0.32 to 2.8 fold those of the relevant single dosing groups (Groups 13, 14 and 15). Antibodies were detected when an adjuvant (alum, or CpG-24555 (U.S. Provisional Patent Application No. 61/121,022, filed Dec. 9, 2008) or ABISCO-100 (Isconova) with CpG-24555) or no adjuvant was used. Antibodies against the peptides were not detected in the untreated controls.

The selectivities of the antibodies elicited by the phosphorylated (A-1P, B-1P, D-1P, C-1P, E-1P) used for immunization were examined in select groups. This was done by determining the antibody titer to the non-phosphorylated versions of the phosphorylated peptides for Groups 1-7 (FIG. 4). The ratio of specific versus nonspecific titer was calculated. In this experiment, the antibodies were selective (>10 fold titer ratio) for B-1P over B-1 in all dosing groups. The antibodies were selective for C-1P over C-1 only in Group 6, the non-alum containing group. The antibodies were selective for A-1P over A-1 in Groups 2, 3, and 6, but not in Group 1, the high dose combination immunization adjuvanted with alum. Antibodies against the non-phosphorylated peptides were not detected in the untreated controls.

### Example 8

### Peptide-VLP Immunization Study for Route, Adjuvant and Isotype

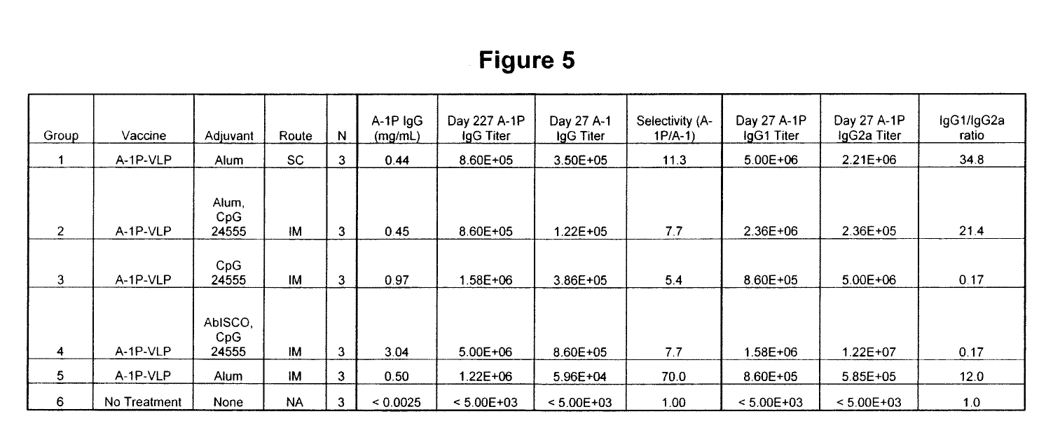

An experiment was performed to compare immunogenicity and the isotype of the antibodies elicited when different adjuvants and routes of administration are used. Groups of 3 Balb/c mice were primed on day 0 and boosted on day 17 as shown in FIG. 5. Sera were collected on day 24. Antibody responses from immunized animals were investigated using the antigen specific titer determination assay, as described in Example 13.

A-1P conjugated to Qbeta-VLP was delivered to BALB/c via subcutaneous or intramuscular injection. Different combinations of antigens were also tested via the intramuscular route. The results using day 27 samples are summarized in FIG. 5. Both the subcutaneous and the intramuscular administrations of A-1P conjugated to Qbeta-VLP and adjuvanted with alum elicited an IgG antibody response. The intramuscularly dosed group had a larger ratio of A-1P to A-1 titer (70) than the subcutaneously dosed group (11). This indicates that route of administration could affect selectivity of response.

As indicated in FIG. 5, all adjuvant combinations used elicited IgG1 and IgG2a antibodies with the alum containing groups (ratio of 21 and 12 for Groups 2 and 5 respectively) having a much greater IgG1 to IgG2a ratio than Groups 3 (0.17) and 4 (0.17), which did not include alum as an adjuvant. This is consistent with known effects of alum to skew immune response to Th2 (see Lindblad, Immunol Cell Biol. 82 (5):497-505 (2004); Marrack et al., Nature Rev. 9:287-293 (2009)). These results suggest that adjuvants can be used to alter the antibody response to the vaccines used in this example. Antibodies against the peptides were not detected in the untreated controls.

### Example 9

### Peptide-VLP Immunization for Linker Analysis

An experiment was performed to determine if immunogenicity is affected by the position of the linker (CGG or GGC) of select peptides from Table 5. Here, the A-1P peptide was used with the linker was on the N-terminus (i.e. SEQ ID NO:31—A-1P) or C-terminus (i.e. SEQ ID NO:41—A-11P) of the peptide. Groups of 4 TG4510+/+ mice were primed on day 0 and boosted on day 14, as shown below in Table 1. Mice were bled on day 20. Antibody responses from immunized animals were investigated using the antigen specific titer determination assay as described in Example 13.

Based on the results shown in Table 1, the linker sequence to the Qbeta-VLPs can be placed on the N- (CGG) or C-terminus (GGC) of the tau specific sequence and still elicit a phosphorylation selective IgG response (>10 fold titer ratio, Table 1). The peptides used in this experiment (SEQ ID NOs:31 and 41) have the same sequence except that the CGG linker is N-terminal in SEQ ID NO:31, and the linker GGC is C-terminal in SEQ ID NO:41. Both elicited a similar IgG titer in the day 20 samples. The antibodies elicited by the two peptide sequences were selective as determined by phosphorylated versus non-phosphorylated IgG titer ratios of 49 and >132, as shown in Table 1. Antibodies against the peptides were not detected in the day 56 untreated controls (Group 7 in FIG. 4).

TABLE 1
Mice were immunized intramuscularly. 100 μg of peptide-VLP and 750 μg
of Alum (Al(OH)₃) were used. Serum dilutions tested in the antigenic specific
titer determination assay (see Example 13) ranged from 1:5,000 to 1:15,800,000.

| | | | | Day 20 IgG Titer | | | |
|---|---|---|---|---|---|---|---|
| | | | | A-1P | | | |
| | | | | IgG | A-1P | | Selectivity |
| Vaccine | Adjuvant | Mouse | N | (mg/mL) | Titer | A-1 Titer | A-1P/A-1 |
| A-1P-VLP | Alum | TG4510++ | 4 | 0.62 | 6.85E+05 | 1.90E+04 | 49.0 |
| A-11P-VLP | Alum | TG4510++ | 4 | 0.42 | 6.58E+05 | <5.00E+03 | >132 |

### Example 10

### Binding of Polyclonal Antibodies to Truncated Peptides

An experiment was performed to determine if select peptides from Table 5 contained the immunogenic epitopes present within A-1P, B-1P or C-1P to which antibodies are elicited. Sera were collected from mice vaccinated with A-1P, B-1P or C-1P as shown below in Table 2. Antibody responses from immunized animals were investigated using the antigen specific titer determination assay (as described in Example 13) with the following modification to the data analysis: a signal twice that of the uncoated well average was considered positive while a signal below twice that of the uncoated well average was considered negative.

In order to determine if antibodies from animals immunized with peptide-VLP conjugates of either A-1P, B-1P or C-1P peptides would bind to shorter versions of each of those peptides, an ELISA was performed. Each tau peptide tested was used as a plate antigen and sera at dilutions of 1:4×10⁴ and 1:4×10⁵ from A-1P-, B-1P- or C-1P-VLP immunized mice were tested to determine if they could bind to the relevant peptide (see Table 3). These sera were previously shown to have antigen specific antibodies. Sera were from mice immunized with the relevant parental peptide (A-1P for A-1P and derivatives, B-1P for B-1P and derivatives, C-1P for C-1P and C-1P/E-1P derivatives) (see Table 2). Each antisera was used at 2 dilutions (1:4×10⁴ and 1:4×10⁵). If binding to the peptide was detected, a positive result is listed. If signal was not detected from either serum dilution, a negative result is listed. All of the samples tested, except for A-5P, A-10P and B-2P, had positive signals, indicating that antibodies elicited by the full length (parent) peptides also bind to most of the shorter derivatives tested.

TABLE 2
Mice were immunized intramuscularly. 100 μg of peptide, 100 μg
of peptide-VLP and 750 μg of Alum (Al(OH)₃) were used
where listed. Dilutions of 1:4 × 10⁴ and 1:4 × 10⁵ were tested
in the antigenic specific titer determination assay (Example 13)
for each serum.

| | Prime | | | | Serum |
|---|---|---|---|---|---|
| | (Day 0) | Boost | | Mouse | Collection |
| Serum | Vaccine | Vaccine | Day(s) | Strain | (Day) |
| 1 | A-1P-VLP + | A-1P-VLP + | 14, 28 | TG4510 −/− | 42 |
| | Alum | Alum | | | |
| 2 | A-1P-VLP + | A-1P-VLP + | 14 | TG4510 −/− | 20 |
| | Alum | Alum | | | |
| 3 | B-1P | B-1P-VLP | 28, 56 | Balb/c | 70 |
| 4 | B-1P | B-1P-VLP | 28, 56 | Balb/c | 70 |
| 5 | C-1P-VLP + | C-1P-VLP + | 14, 28 | TG4510 −/− | 42 |
| | Alum | Alum | | | |
| 6 | C-1P | C-1P-VLP | 28, 56 | Balb/c | 70 |

TABLE 3
“Positive” indicates that the OD for that well was at least twice
that of the OD of the background (uncoated well) average. “Negative”
indicates that the OD for that well was less than twice that of the OD
of the background (uncoated well) average.

| Peptide | Serum A | Serum B |
|---|---|---|
| A-1P | Positive | Positive |
| A-2P | Positive | Positive |
| A-4P | Positive | Negative |
| A-5P | Negative | Negative |
| A-6P | Positive | Positive |
| A-7P | Positive | Positive |
| A-8P | Positive | Positive |
| A-9P | Positive | Positive |
| A-10P | Negative | Negative |
| B-1P | Positive | Positive |
| B-2P | Negative | Negative |
| B-3P | Positive | Positive |
| B-4P | Positive | Negative |
| B-5P | Positive | Negative |
| B-6P | Positive | Negative |
| C-1P | Positive | Positive |
| C-2P | Positive | Positive |
| C-3P | Positive | Positive |
| C-4P | Positive | Positive |
| C-5P | Positive | Positive |

### Example 11

### Truncated Peptide Immunization Study for Immunogenicity and Memory

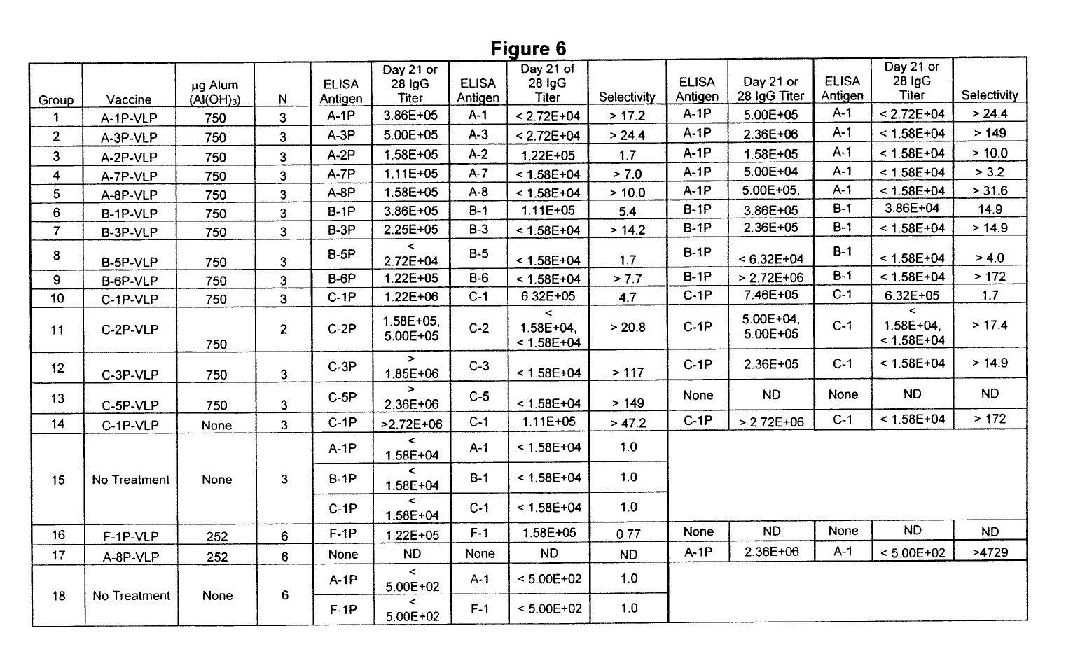

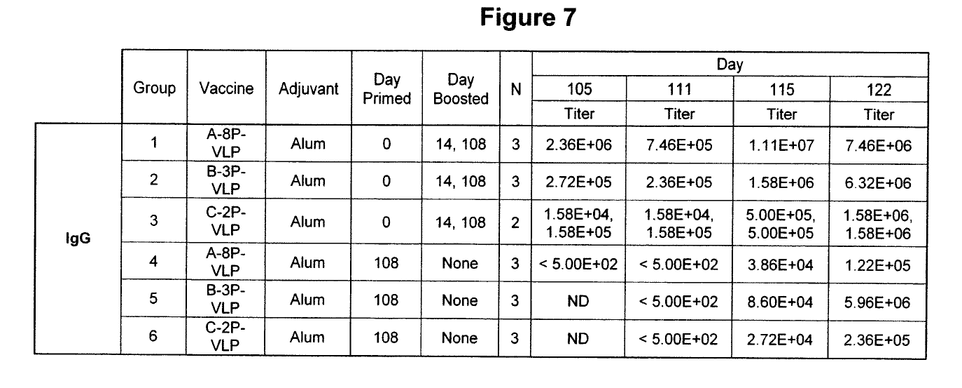

Two experiments were performed to determine if select peptides from Table 5 were immunogenic when immunized as Qbeta-VLP conjugates. One of these studies was also used to determine if immunologic memory developed. In an effort to avoid potential binding of peptide antigens to MHC Class I and MHC class II T-cell ligands, shorter versions of the A-1P, B-1P and C-1P ‘parent’ peptides were tested. Peptide lengths of 7 to 11 amino acids were chosen since MHC Class II molecules generally bind peptides with 13-17 amino acids and a peptide length of at least 8 amino acids is required for MHC I binding (Murphy et al., Janeway's Immunobiology, Garland Science (2007)). Therefore, peptides having 11 or fewer amino acids should not induce a MHC class II restricted CD4 T-cell response and 7 amino acid peptides should induce neither a CD4 T-cell nor a MHC class I restricted CD8 T-cell response. Peptide F-1P, with a length of 7 amino acids, was also tested. Groups of 3 or 6 Balb/c mice were primed on day 0 and boosted on day 14 as shown in FIG. 6. Three groups were also boosted on day 108 and three groups were primed on day 108 (see FIG. 7). Sera were collected on day 21, or day 28, or days 111, 115, and 122 or days 21, 105, 111, 115 and 122. Antibody responses from immunized animals were investigated using the antigen specific titer determination assay (as described in Example 13).

The results are summarized in FIG. 6. All peptide-Qbeta-VLP conjugates elicited antigen specific IgG antibodies from all of the mice tested in the ELISA except for B-5P in which only 2 of the 3 mice had detectable antibodies at a serum dilution of 1:15,800. These results indicate that the 7-11 amino acid tau peptides with a CGG linker are immunogenic and can elicit antibodies specific to the immunogen.

The selectivities of the antibodies elicited to the phosphorylated peptide form used for immunization were examined (see FIG. 6). Most of these peptides were selective (>10 fold titer ratio) for the phosphorylated form of the peptide over the non-phosphorylated form. Many of the shortened A-1P, B-1P and C-1P derivates had no ELISA signal detected when the non-phosphorylated version of the immunizing peptide was used as the plate antigen. The selectivity of many of the shortened A-1P, B-1P and C-1P derivatives is equal to or greater than the parent peptide. Active immunization of peptide A-2P without the CGG linker has been reported to reduce aggregated Tau in the brain and slow progression of tangle-related sensorimotor impairments in the JNPL3 Tau P301L over-expressing animal model (Asuni et al., J. Neurosci. 27:9115 (2007)). A-2P, when conjugated to Qbeta-VLP, was immunogenic. The elicited antibodies, however, were not selective for the phosphorylated version of the peptide (A-2P) relative to the non-phosphorylated version (A-2) in the ELISA assay (A-2P/A-2 titer ratio of 1.7). In contrast, these antibodies were selective for A-1P over A-1 (A-1P/A-1 titer ratio of >10.0). The titers, when using A-2P and A-1P as ELISA antigens, were the same. This suggests the epitopes of most of the non-phosphospecific antibodies include the 12 amino acids of peptide A-2P which are not contained in A-1P. In this experiment, C-1P had greater selectivity when tested without alum as an adjuvant than with alum (Groups 14 and 10 respectively). Adjuvants, such as alum, can be used to modify the selectivity for the phosphorylated versus the non-phosphorylated peptide. Antibodies against the peptides were not detected in the untreated controls. These results indicate that the 7-11 amino acid tau peptides with a CGG linker can elicit phospho-peptide selective antibodies.

The results testing for a memory recall response for A-1P, B-1P and C-1P are shown in FIG. 7. The day 111, 115 and 122 IgG titers (days +3, +7 and +14 from last immunization, respectively) for peptide-Qbeta-VLP immunized mice which were primed and boosted on days 0, 14 and 108 were compared to those of mice primed on day 108. Groups 1, 2 and 3 had large IgG titers on day 105, 84 days after last boost. In comparison to the day 108 prime groups (Groups 4, 5 and 6), these groups also had large increases in IgG titer between days 111 and 115. These data are indicative of a long term antibody response and memory recall.

### Example 12

### Truncated Peptide Immunization Study for Immunogenicity and T-Cell Response in Combination with and without Alum

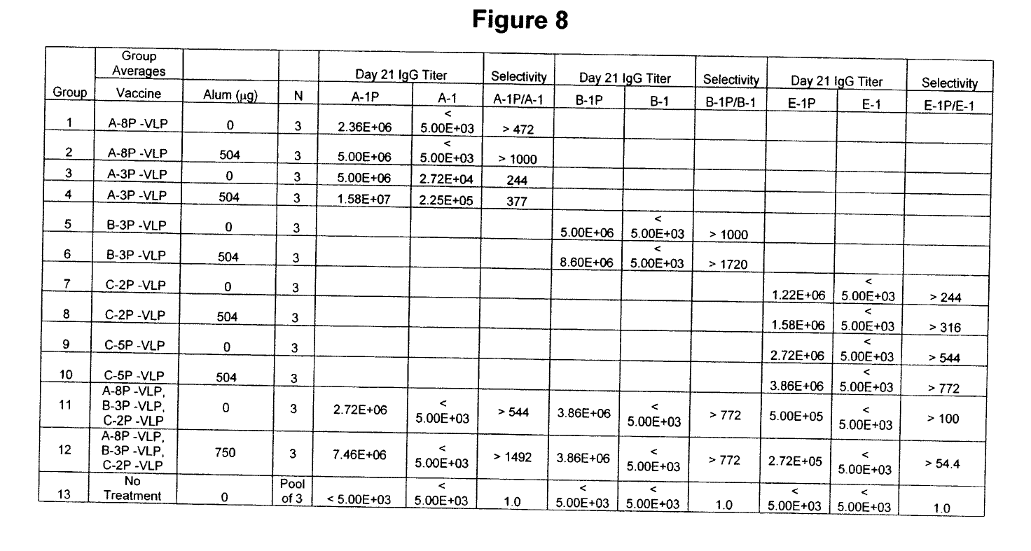

An experiment was performed to determine if peptides derived from A-1P, B-1P and C-1P (Table 5) were immunogenic when immunized with 100 μg of a Qbeta-VLP conjugate with 0 or 504 μg of alum (Al(OH)₃) or when given as a combination of peptide-Qbeta-VLP conjugates with or without alum. T-cell responses in the spleen were also analyzed. Groups of 3 TG4510−/− wild-type littermate mice were primed on day 0 and boosted on day 14 as shown in FIG. 8. Sera and spleens were collected on day 21. Antibody responses from immunized animals were investigated using the antigen specific titer determination assay (as described in Example 13) and the IFN-γ ELISPOT assay (as described in Example 14).

The antigen specific IgG titers show that the peptides tested were all immunogenic when immunized as a Qbeta-VLP conjugate with 504 μg of alum (Al(OH)₃) or without alum (see FIG. 8). Immunization of A-8P, B-3P and C-2P as a combination with a total of 750 μg alum to 300 μg peptide-Qbeta-VLP conjugates resulted in a selective antibody response to all 3 of the peptides.

The selectivity of the antibodies elicited to the immunized phosphorylated peptide versus the non-phosphorylated version of that peptide was examined by ELISA (FIG. 8). The ratio of specific versus non specific titer was calculated with a larger ratio indicating greater selectivity. The antibodies elicited were selective for the phosphorylated form of the peptide whether alum was included in the prime and boost or not, and whether the peptide-Qbeta-VLP conjugates were immunized alone or in combination.

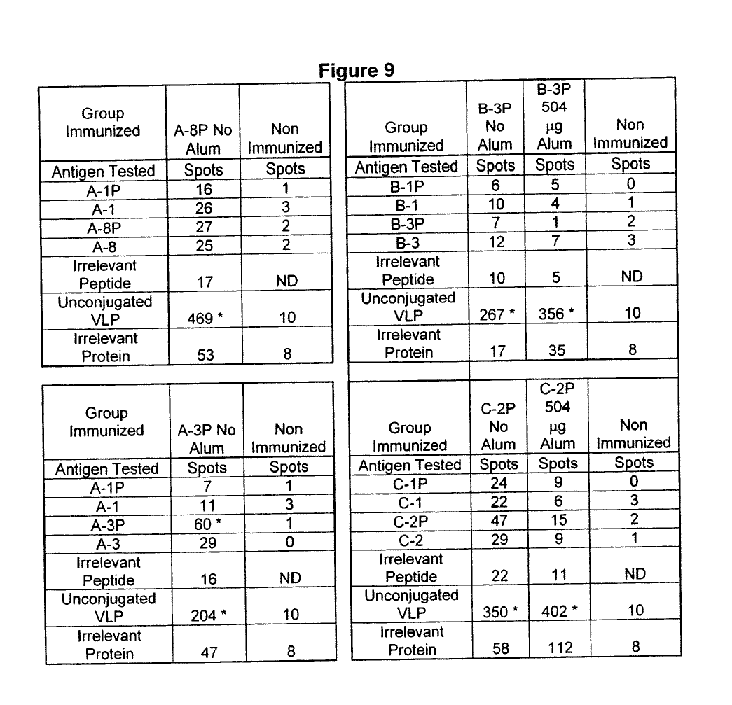

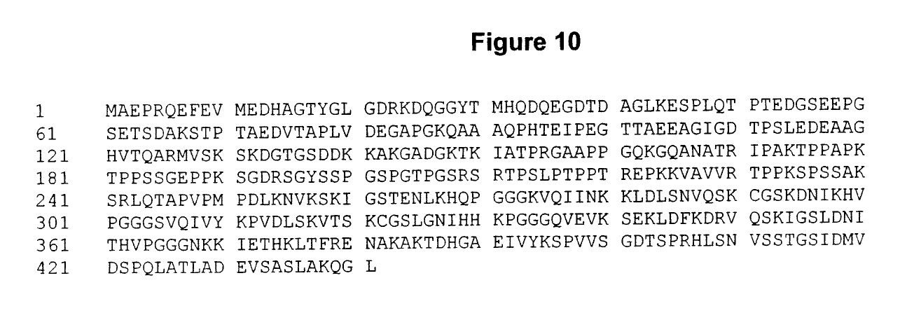

T-cell responses in the spleen after immunization with a single peptide Qbeta-VLP were analyzed using IFN-γ ELISPOT analysis (see FIG. 9). The frequency of T-cells secreting IFN-γ specific to the parent tau peptides (A-1P, B-1P, C-1P) and their corresponding truncated versions were analyzed on day 21, 7 days after the last peptide Qbeta-VLP boost. Relative to an irrelevant peptide control (HBV-1), no significant numbers of B-1P, B-1, B-3P, B-3, C-1P, C-1, C-2P or C-2 specific IFN-γ secreting T-cells were generated after immunization with B-3P-Qbeta-VLP and C-2P-Qbeta-VLP either in the presence or absence of alum. Significant (p<0.05) levels of A-3P specific IFN-γ T-cell responses were induced after immunization with A-3P-Qbeta-VLP. The A-3P peptide contains a predicted mouse MHC Class I K^{b} binding epitope (IVYKSVV, see Lundegaard et al. Bioinformatics 24:1397-1398 (2008)), and this epitope could contribute to the T-cell response observed in A-3P immunized animals. This epitope is also present in A-1P, A-1, A-2P, A-2 and A-3. When the A-1P peptide was shortened to a 7 amino acid length peptide (A-8P Qbeta-VLP), IFN-γ specific T-cell responses in A-8P Qbeta-VLP immunized mice were reduced to background levels.

CD4 T helper cells are required for the generation of isotype switched antibody responses and the generation of memory B cells (see Murphy et al., Janway's Immunobiology, Garland Science, (2007)). Thus, the finding that IgG antibody responses were generated to their respective peptide epitopes after immunization with truncated phospho-tau peptide Qbeta-VLP suggests that CD4 T helper responses are induced against the vaccine. Since no significant levels of tau-peptide specific T-cells were generated after immunization with the truncated peptide conjugates, a T-cell response to another component of the vaccine was tested for. Analysis of T-cell responses to the VLP protein shows that IFN-γ specific T-cells were generated against VLP epitopes (4-15 fold over irrelevant protein control (BSA, Sigma Aldrich A9418).

### Example 13

### Antigen Specific Antibody Titer Determination

The following assay was used to determine the antibody responses from immunized animals, as described above in Examples 5-12.

A colormetric ELISA was used to determine the highest dilution of serum which had detectable antigen specific antibodies, as represented by a positive signal. Serial dilutions were prepared from sera samples and tested in the assay. In some assays, monoclonal antibodies specific to the phospho-tau peptide were used as positive controls or standards. Sera from un-vaccinated mice (BALB/c, TG4510+/+ or Tg4510−/−) were used as a negative control. 96-well high binding polystyrene plates (CoStar 9018) were coated with 100 μL peptide diluted in 0.1M sodium carbonate pH 8.2 (Sigma S7795) at 4° C., for 18 to 21 hours. All of the peptides were at a concentration of 0.3 μg/mL except for C-1P and C-1 which were at 3 μg/mL. The following day, the coating solution was removed and the plates were blocked with a solution of PBS (EMD OmniPure 6507) containing 0.05% Tween 20 (Sigma P2287) and 1% BSA (Sigma A9418) shaking using Heildolph Titramax 1000 at 600 rpm for 1 hour at room temperature. The blocking solution was removed before the samples were added to the plates.

Mouse sera and monoclonal antibodies used as standards were serially diluted using 0.5 or 1 log dilutions in PBS containing 0.5% Tween 20 (PBS-T). Six or eight dilutions, starting at 1:500, 1:5000 or 1:15,800. for the serum samples, were tested for each sample. The monoclonal antibodies used as standards and positive controls were: anti-Tau 396 (Zymed 35-5300) for A-1P, AT-180 (Thermo Pierce MN1040) for B-1P; AT-8 (Thermo Pierce MN1020) for D-1P and E-1P; AT-100 (Thermo Pierce MN1060) for C-1P. 50, 15.8, 5, 1.58, 0.5, 0.158 and 0.05 ng per well were the concentrations used of the monoclonal antibodies for the standard curve.

The samples and standards were added to the plates at 100 μL per well in duplicate wells. The plates were incubated for 1 hour at room temperature, shaking at 600 rpm. The plates were then washed 3 times with PBS-T and the secondary antibody (HRPO-conjugated anti-mouse IgG, Caltag #M30107) diluted to 1:3000 in PBS-T was added at 100 μL/well. Different secondary antibodies were used for detecting IgG₁ (Caltag #M32107 1:2000), IgG₂ₐ (Caltag #M32307 1:2000), and IgM (Caltag #31507 1:3000). The secondary antibody was allowed to bind on the plates for 1 hour at room temperature with shaking. The plates were again washed 3 times with PBS-T and the plates were blotted dry after the final wash. To develop, 100 μL TMB Peroxidase EIA Substrate (Bio-Rad #172-1067) was added to each well for 11 minutes at room temperature. To stop the reaction, 100 μL 1N sulfuric acid was added to each well. The absorbance was read at 450 nm on a Molecular Devices Spectramax plus 384. An OD threshold value was calculated for each plate by taking the average of all wells treated with PBS-T and adding 3 times the standard deviation of those wells. If a standard deviation could not be calculated, then a value of twice the PBS-T OD was used as the threshold value. The sample titer was determined from the first sample dilution with a 450 nm absorbance value greater than the calculated threshold value. For some assays, a standard curve based on dilutions of the relevant positive control monoclonal was used to calculate titer concentration relative to the standard curve. The value of the lowest dilution or standard tested was used for calculations when no signal was detected and the value of the largest dilution or standard tested was used when the largest dilution was positive. Mean titers were calculated when N was greater than 2 while individual values were shown when N was 1 or 2. Selectivity ratios were determined by dividing the sample titer for a phosphorylated peptide by the titer of the non-phosphorylated version of the same peptide for each sample, then averaging the ratio for the different samples. Values of greater than 10 or less than 0.1 were considered selective. Using the first positive dilution to determine selectivity was the most conservative method. Using other methods, such as a threshold OD of 1 or half maximal OD, will likely give larger selectivity values.

### Example 14

### IFN-γ ELISPOT Assay

The IFN-γ ELISPOT kit (BD Biosciences; 551083) was used to measure T-cell responses after immunization with peptide-Qbeta-VLP. ELISPOT was performed on pooled spleens (N=3) from A-8P, A-3P, B-3P, C-2P (in the presence of low dose alum or no alum) immunized mice and also non-immunized mice. 96 well ELISPOT plates were plated with 5 μg/mL of capture anti-mouse IFN-γ antibody overnight at 4° C. Antibody coated plates were washed and blocked with RPMI 1640 complete medium (Invitrogen 11875-119) containing 10% fetal bovine serum (VWR A15-204).

Splenocytes were then seeded into anti-IFN-γ antibody coated plates at 500,000 splenocytes per well stimulated with 10 μg/mL of peptide or protein antigen for 20 to 24 hours in a 37° C. incubator with 5% CO₂. The irrelevant peptide control was peptide HBV-1 (SEQ ID NO:77) and Bovine Serum Albumin (Sigma Aldrich; A9418) was used as an irrelevant protein control for Qbeta-VLP. Phorbol 12-Myristate 13-Acetate (0.5 μg/mL PMA, Sigma Aldrich; P8139) and ionomycin (0.5 μg/mL, Sigma Aldrich; 10634) stimulation of spleen cells seeded at 55,555 and 18,520 cells per well were used as positive controls. After the 20 to 24 hour incubation period, ELISPOT plates were washed twice with distilled water, followed by an additional three washes with wash buffer (1×PBS (Invitrogen 10010072) containing 0.05% Tween-20 (Sigma P2287)). Detection of IFN-γ cytokine was done by incubating 2 μg/mL of biotinylated anti-IFN-γ detection antibody diluted in PBS containing 10% FBS for 2 hours at room temperature, followed by incubation with 1:100 of Streptavidin HRP diluted in PBS 10% FBS. After washing plates 4 times with wash buffer and 2 times with PBS, IFN-γ spots were visualized using AEC chromagen-substrate (11 minute incubation at room temp).

IFN-γ positive spots were scanned, captured, and counted using the Cellular Technology ELISpot analyzer and the 5.0 Professional Immunospot software and mean counts per well. The irrelevant peptide was the negative control for the peptide antigens while BSA was the negative control for unconjugated VLP. To be considered positive, the mean spots value must be significantly greater (p<0.05) than the relevant negative control using the Student's T-Test.

### Example 15

### Adjuvant Formulation and Immunization

Adjuvants used in the specific examples described herein (e.g. Examples 5-14) were prepared as follows. CpG-24555 was made into a 2 mg/mL stock in water. Alum used was Alhydrogel “85” (Brenntag Biosector) containing 10 mg/mL aluminum. Alhydrogel “85” was mixed at a 1:1 ratio with 100 μg of peptide or VLP conjugated peptide. Generally, up to 25 μL (for intramuscular vaccinations) or 50 μL (for subcutaneous vaccinations) was added to solution with 100 μg of VLP and immediately vortexed and placed on ice. TiterMax Gold (Alexis Biochemicals) was added at a ratio of 1:1 with peptide solutions. 50 μL of TiterMax Gold was added to 50 μL of a 2 mg/mL peptide solution for a 100 μL subcutaneous dose and emulsified for 10 minutes at 4° C. with a Mixermill (SPEX Sample Prep). 25 μL (12 μg) of AbISCO-100 (Isconova) was added to up to 100 μg of VLP-peptide solution and 5 μL (10 μg) of CpG-24555, vortexed and placed on ice.

Immunization and animal work performed in the specific examples described herein (e.g. Examples 5-14) was carried out according to generally accepted methods. For vaccinations, up to 100 μL of vaccine was injected subcutaneously at the base of the tail or 50 μL injected into one or both of the rear anterior tibiallis muscle. Blood collection was performed via sub-mandibular lancing or terminally via cardiac puncture. Spleens were removed post exsanguination and cervical dislocation and placed in cold sterile HBBS (Invitrogen Cat #14170) with 5% PBS and Penn/Strep (Invitrogen Cat. # 15140-122). Spleens were mashed on a 70 μm screen (Falcon). Cells were washed in ice cold HBBS and red blood cells were lysed with ACK lysis buffer (Invitrogen). Splenocytes were counted on a Guava PCA 96 (Guava Technologies Inc.).

### Example 16

### Optimization of pTau Peptide Conjugation Density to Qbeta/VLP for Desired Immune Response

An experiment was performed to determine if pTau peptide epitope conjugation density to Qbeta/VLP (number of peptides per Qbeta monomer subunit) affected the pTau specific antibody response. Different coupling conditions, produced by varying the molar excess of SMPH and the pTau peptide excess, were used to produce 8 pTau/VLP conjugates of different epitope densities (Table 4). Groups of 5 female BalbC mice (8 weeks old) were immunized on day 0 and day 14 (sc) with 100 ug of each of the different density conjugates in 750 μg of Alum (Al(OH)₃). Serum was collected on day 26. Antibody responses from immunized animals were investigated using the antigen specific titer determination assay as described in Example 13.

Based on the Day 26 titer results shown in Table 4, the 2.3 conjugation density for A-8P/QBeta produced a higher titer immune response than the higher (3.6) density conjugated form. For the different B-3P/Qbeta conjugates, titers were similar and highest for the 2.2 and 3.6 conjugate density forms. For C-2P/Qbeta, the 2.2 and 3.5 epitope conjugation densities produced similar titers, which were slightly higher than the 4.3 conjugation density form. The results show that epitope conjugation density can affect the antibody response in an antigen specific manner and that, in general, coupling conditions resulting in a conjugation density of 2-3 pTau peptide epitopes per Qbeta monomer are preferred.

TABLE 4
Mice were immunized subcutaneously with 10 or 100 μg of the indicated different coupling density pTau-peptide/Qbeta/VLP
conjugates in 750 μg of Alum (Al(OH)₃) on days 0 and 14. Serum dilutions from day 26 were tested in the
antigenic specific titer determination assay described in Example 13. Titer results are indicated.

| | | A-8P/Qbeta | | B-3P/Qbeta | | | C-2P/Qbeta | | |
|---|---|---|---|---|---|---|---|---|---|
| | | Group 1 | Group 2 | Group 3 | Group 4 | Group 5 | Group 6 | Group 7 | Group 8 |
| Derivatization | SMPH Excess | 10X | 40X | 7.5X | 20X | 80X | 7.5X | 20X | 80X |
| Coupling | Peptide Excess | 5X | 10X | 5X | 10X | 10X | 5X | 10X | 10X |
| Conjugation Density | | 2.3 | 3.6 | 2.2 | 3.6 | 4.4 | 2.2 | 3.5 | 4.3 |
| Day 26 IgG Titer | | 9.00E+04 | 3.00E+04 | 1.50E+05 | 1.20E+05 | 8.00E+04 | 1.00E+05 | 1.80E+05 | 4.00E+04 |

TABLE 5
Summary of Sequence Listing
In the following table, and as noted previously herein,
phosphorylated amino acids are indicated as bold and
underlined.

| SEQ | | |
|---|---|---|
| NO: | DESCRIPTION | SEQUENCE |
| 1 | pThr-231/pSer-235 | TPPKS |
| | phospho-tau epitope | |
| 2 | Alternative pThr-231/pSer-23 | PPKS |
| | phospho-tau epitope | |
| 3 | pSer-202/pThr-205 | SPGT |
| | phospho-tau epitope | |
| 4 | Peptide A-1P without linker | EIVYKSPVVSGDTSPRHLS |
| 5 | Peptide A-2P without linker | RENAKAKTDHGAEIVYKSPVVSGDTSPRHLS |
| 6 | Peptide A-3P without linker | EIVYKSPVVS |
| 7 | Peptide A-4P without linker | GDTSPRH |
| 8 | Peptide A-5P without linker | KSPVVSGDTSP |
| 9 | Peptide A-6P without linker | EIVYKSP |
| 10 | Peptide A-7P without linker | IVYKSPV |
| 11 | Peptide A-8P without linker | VYKSPVV |
| 12 | Peptide A-9P without linker | YKSPVVS |
| 13 | Peptide A-10P without linker | KSPVVSG |
| 14 | Peptide B-1P without linker | KVAVVRTPPKSPSSAKS |
| 15 | Peptide B-2P without linker | VRTPPKSPS |
| 16 | Peptide B-3P without linker | VVRTPPKSP |
| 17 | Peptide B-4P without linker | RTPPKSPSS |
| 18 | Peptide B-5P without linker | RTPPKSP |
| 19 | Peptide B-6P without linker | PPKSPSS |
| 20 | Peptide C-1P without linker | SRSRTPSLPTPPT |
| 21 | Peptide C-2P without linker | SRTPSLP |
| 22 | Peptide C-3P without linker | RTPSLPT |
| 23 | Peptide C-4P without linker | RSRTPSL |
| 24 | Peptide C-5P without linker | PGSRSRTPSLP |
| 25 | Peptide D-1P without linker | GYSSPGSPGTPGSRS |
| 26 | Peptide E-1P without linker | GYSSPGSPGTPGSRSRTPSLPTPPT |
| 27 | CpG 7909 | 5′ TCGTCGTTTTGTCGTTTTGTCGTT 3′ |
| 28 | CpG 10103 | 5′ TCGTCGTTTTTCGGTCGTTTT 3′ |
| 29 | CpG 24555 | 5′ TCGTCGTTTTTCGGTGCTTTT 3′ |
| 30 | Human tau isoform 2 | Genbank Accession No. NP_005901 |
| 31 | Peptide A-1P with linker | CGGEIVYKSPVVSGDTSPRHLS |
| 32 | Peptide A-2P with linker | CGGRENAKAKTDHGAEIVYKSPVVSGDTSP |
| | | RHLS |
| 33 | Peptide A-3P with linker | CGGEIVYKSPVVS |
| 34 | Peptide A-4P with linker | CGGGDTSPRH |
| 35 | Peptide A-5P with linker | CGGKSPVVSGDTSP |
| 36 | Peptide A-6P with linker | CGGEIVYKSP |
| 37 | Peptide A-7P with linker | CGGIVYKSPV |
| 38 | Peptide A-8P with linker | CGGVYKSPVV |
| 39 | Peptide A-9P with linker | CGGYKSPVVS |
| 40 | Peptide A-10P with linker | CGGKSPVVSG |
| 41 | Peptide A-11P with linker | EIVYKSPVVSGDTSPRHLSGGC |
| 42 | Peptide B-1P with linker | CGGKVAVVRTPPKSPSSAKS |
| 43 | Peptide B-2P with linker | CGGVRTPPKSPS |
| 44 | Peptide B-3P with linker | CGGVVRTPPKSP |
| 45 | Peptide B-4P with linker | CGGRTPPKSPSS |
| 46 | Peptide B-5P with linker | CGGRTPPKSP |
| 47 | Peptide B-6P with linker | CGGPPKSPSS |
| 48 | Peptide C-1P with linker | CGGSRSRTPSLPTPPT |
| 49 | Peptide C-2P with linker | CGGSRTPSLP |
| 50 | Peptide C-3P with linker | CGGRTPSLPT |
| 51 | Peptide C-4P with linker | CGGRSRTPSL |
| 52 | Peptide C-5P with linker | CGGPGSRSRTPSLP |
| 53 | Peptide D-1P with linker | CGGYSSPGSPGTPGSRS |
| 54 | Peptide E-1P with linker | CGGYSSPGSPGTPGSRSRTPSLPTPPT |
| 55 | Peptide A-1 with linker | CGGEIVYKSPVVSGDTSPRHLS |
| 56 | Peptide A-2 with linker | CGGRENAKAKTDHGAEIVYKSPVVSGDTSP |
| | | RHLS |
| 57 | Peptide A-3 with linker | CGGEIVYKSPVVS |
| 58 | Peptide A-4 with linker | CGGGDTSPRH |
| 59 | Peptide A-5 with linker | CGGKSPVVSGDTSP |
| 60 | Peptide A-6 with linker | CGGEIVYKSP |
| 61 | Peptide A-7 with linker | CGGIVYKSPV |
| 62 | Peptide A-8 with linker | CGGVYKSPVV |
| 63 | Peptide A-9 with linker | CGGYKSPVVS |
| 64 | Peptide A-10 with linker | CGGKSPVVSG |
| 65 | Peptide B-1 with linker | CGGKVAVVRTPPKSPSSAKS |
| 66 | Peptide B-2 with linker | CGGVRTPPKSPS |
| 67 | Peptide B-3 with linker | CGGVVRTPPKSP |
| 68 | Peptide B-4 with linker | CGGRTPPKSPSS |
| 68 | Peptide B-5 with linker | CGGRTPPKSP |
| 69 | Peptide B-6 with linker | CGGPPKSPSS |
| 70 | Peptide C-1 with linker | CGGSRSRTPSLPTPPT |
| 71 | Peptide C-2 with linker | CGGSRTPSLP |
| 72 | Peptide C-3 with linker | CGGRTPSLPT |
| 73 | Peptide C-4 with linker | CGGRSRTPSL |
| 74 | Peptide C-5 with linker | CGGPGSRSRTPSLP |
| 75 | Peptide D-1 with linker | CGGYSSPGSPGTPGSRS |
| 76 | Peptide E-1 with linker | CGGYSSPGSPGTPGSRSRTPSLPTPPT |
| 77 | Peptide HBV-1 | IPQSLDSWWTSL |
| 78 | 3′ sequence of Qbeta | 5′-GTATTAATGACTCGAG-3′ |
| | containing a XhoI site | |
| 79 | Linker | GGGGGC |
| 80 | Linker | GGGGC |
| 81 | Linker | GGGC |
| 82 | Linker | GGGGGK |
| 83 | Linker | GGGGK |
| 84 | Linker | GGGK |
| 85 | Linker | GGGGSC |
| 86 | Linker | GGGSC |
| 87 | Linker | GGSC |
| 88 | Linker | CSGGGG |
| 89 | Linker | CSGGG |
| 90 | Linker | CSGG |
| 91 | Linker | CGGGG |
| 92 | Linker | CGGG |
| 93 | Linker | CGGGGG |
| 94 | Linker | CGDKTHTSPP |
| 95 | Linker | DKTHTSPPCG |
| 96 | Linker | CGGPKPSTPPGSSGGAP |
| 97 | Linker | PKPSTPPGSSGGAPGGCG |
| 98 | Linker | GCGGGG |
| 99 | Linker | GGGGCG |
| 100 | Linker | CGKKGG |
| 101 | Linker | CGDEGG |
| 102 | Linker | GGKKGC |
| 103 | Linker | GGEDGC |
| 104 | Linker | GGCG |
| 105 | Peptide F-1P without linker | AGTYGLG |
| 106 | Peptide F-1P with linker | CGGAGTYGLG |
| 107 | Peptide F-1 with linker | CGGAGTYGLG |
| 108 | Peptide F-2P without linker | DHAGT [symbol] G |
| 109 | Peptide F-3P without linker | HAGTYGL |
| 110 | Peptide F-4P without linker | GTYGLGD |
| 111 | Peptide F-5P without linker | TYGLGDR |
| 112 | Peptide F-6P without linker | DHAGTYGLG DR |
| 113 | Peptide F-2P with linker | CGGDHAGT [symbol] G |
| 114 | Peptide F-3P with linker | CGGHAGTYGL |
| 115 | Peptide F-4P with linker | CGGGTYGLGD |
| 116 | Peptide F-5P with linker | CGGTYGLGDR |
| 117 | Peptide F-6P with linker | CGGDHAGTYGLG DR |
| 118 | Peptide F-2 with linker | CGGDHAGTYG |
| 119 | Peptide F-3 with linker | CGGHAGTYGL |
| 120 | Peptide F-4 with linker | CGGGTYGLGD |
| 121 | Peptide F-5 with linker | CGGTYGLGDR |
| 122 | Peptide F-6 with linker | CGGDHAGTYGLG DR |

We claim:

1. An immunogen comprising at least one antigenic tau peptide linked to an immunogenic carrier, wherein said antigenic tau peptide comprises a phospho-tau epitope selected from a pSer-396 phospho-tau epitope, a pThr-231/pSer-235 phospho-tau epitope, a pThr-231 phospho-tau epitope, a pSer-235 phospho-tau epitope, a pThr-212/pSer-214 phospho-tau epitope, a pSer-202/pThr-205 phospho-tau epitope, and a pTyr 18 phospho-tau epitope.

2. An immunogen comprising at least one antigenic tau peptide linked to an immunogenic carrier, wherein said antigenic tau peptide comprises an amino acid sequence selected from SEQ ID NOs: 4, 6-26, 105 and 108-112.

3. An immunogen according to claim 2, wherein said antigenic tau peptide is covalently linked to said immunogenic carrier by a linker represented by the formula (G)ₙC, where said linker is at either the C-terminus (peptide-(G)ₙC) or N-terminus (C(G)ₙ-peptide) of said peptide, and where n is 0, 1, 2, 3, 4, 5, 6, 7, 8, 9, or 10.

4. An immunogen according to claim 3, wherein said antigenic tau peptide comprises an amino acid sequence selected from SEQ ID NOs: 4 and 6-13.

5. An immunogen according to claim 4, wherein said antigenic tau peptide consists of the amino acid sequence set forth in SEQ ID NO: 11.

6. An immunogen according to claim 3, wherein said antigenic tau peptide comprises an amino acid sequence selected from SEQ ID NOs: 14-19.

7. An immunogen according to claim 6 wherein said antigenic tau peptide consists of the amino acid sequence set forth in SEQ ID NO: 16.

8. An immunogen according to claim 3, wherein said antigenic tau peptide comprises an amino acid sequence selected from SEQ ID NOs:20-24.

9. An immunogen according to claim 8 wherein said antigenic tau peptide consists of the amino acid sequence set forth in SEQ ID NO:21.

10. An immunogen according to claim 3, wherein said antigenic tau peptide comprises an amino acid sequence selected from SEQ ID NOs: 105 and 108-112.

11. An immunogen according to claim 10 wherein said antigenic tau peptide consists of the amino acid sequence set forth in SEQ ID NO:105.

12. An immunogen according to claim 1, wherein said immunogenic carrier is a virus-like particle selected from the group consisting of HBcAg VLP, HBsAg VLP, and Qbeta VLP.

13. A composition comprising at least two immunogens according to claim 2, wherein:
a) the antigenic tau peptide of the first immunogen consists of an amino acid sequence selected from SEQ ID NOs: 4 and 6-13; and
b) the antigenic tau peptide of the second immunogen consists of an amino acid sequence selected from SEQ ID NOs: 14-19.

14. A composition comprising at least two immunogens according to claim 2, wherein:
a) the antigenic tau peptide of the first immunogen consists of an amino acid sequence selected from SEQ ID NOs: 4 and 6-13; and
b) the antigenic tau peptide of the second immunogen consists of an amino acid sequence selected from SEQ ID NOs: 20-24.

15. A composition comprising at least two immunogens according to claim 2, wherein:
a) the antigenic tau peptide of the first immunogen consists of an amino acid sequence selected from SEQ ID NOs: 14-19; and
b) the antigenic tau peptide of the second immunogen consists of an amino acid sequence selected from SEQ ID NOs: 20-24.

16. A composition comprising at least two immunogens according to claim 2, wherein:
a) the antigenic tau peptide of the first immunogen consists of an amino acid sequence selected from SEQ ID NOs: 4 and 6-13; and
b) the antigenic tau peptide of the second immunogen selected from SEQ ID NO: 105 and 108-112.

17. A composition comprising at least two immunogens according to claim 2, wherein:
a) the antigenic tau peptide of the first immunogen consists of an amino acid sequence selected from SEQ ID NOs: 14-19; and
b) the antigenic tau peptide of the second immunogen selected from SEQ ID NO: 105 and 108-112.

18. A composition comprising at least two immunogens according to claim 2, wherein:
a) the antigenic tau peptide of the first immunogen consists of an amino acid sequence selected from SEQ ID NOs: 20-24; and
b) the antigenic tau peptide of the second immunogen selected from SEQ ID NO: 105 and 108-112.

19. A composition comprising at least three of the four immunogens according to claim 2, wherein:
a) the antigenic tau peptide of the first immunogen consists of an amino acid sequence selected from SEQ ID NOs:4, and 6-13;
b) the antigenic tau peptide of the second immunogen consists of an amino acid sequence selected from SEQ ID NOs:14-19; and
c)) the antigenic tau peptide of the third immunogen consists of an amino acid sequence selected from SEQ ID NOs:20-24.
d) the antigenic tau peptide of the fourth immunogen consists of an amino acid sequence selected from SEQ ID NO: 105 and 108-112.

20. A pharmaceutical composition comprising the immunogen of claim 1 and a pharmaceutically acceptable excipient.


SEQUENCE LISTING (123 sequences)

SEQ ID NO: 1 - protein, 5 aa, Artificial Sequence
  Synthetic
TPPKS

SEQ ID NO: 2 - protein, 4 aa, Artificial Sequence
  Synthetic
PPKS

SEQ ID NO: 3 - protein, 4 aa, Artificial Sequence
  Synthetic
SPGT

SEQ ID NO: 4 - protein, 19 aa, Artificial Sequence
  Synthetic
EIVYKSPVVSGDTSPRHLS

SEQ ID NO: 5 - protein, 31 aa, Artificial Sequence
  Synthetic
RENAKAKTDHGAEIVYKSPVVSGDTSPRHLS

SEQ ID NO: 6 - protein, 10 aa, Artificial Sequence
  Synthetic
EIVYKSPVVS

SEQ ID NO: 7 - protein, 7 aa, Artificial Sequence
  Synthetic
GDTSPRH

SEQ ID NO: 8 - protein, 11 aa, Artificial Sequence
  Synthetic
KSPVVSGDTSP

SEQ ID NO: 9 - protein, 7 aa, Artificial Sequence
  Synthetic
EIVYKSP

SEQ ID NO: 10 - protein, 7 aa, Artificial Sequence
  Synthetic
IVYKSPV

SEQ ID NO: 11 - protein, 7 aa, Artificial Sequence
  Synthetic
VYKSPVV

SEQ ID NO: 12 - protein, 7 aa, Artificial Sequence
  Synthetic
YKSPVVS

SEQ ID NO: 13 - protein, 7 aa, Artificial Sequence
  Synthetic
KSPVVSG

SEQ ID NO: 14 - protein, 17 aa, Artificial Sequence
  Synthetic
KVAVVRTPPKSPSSAKS

SEQ ID NO: 15 - protein, 9 aa, Artificial Sequence
  Synthetic
VRTPPKSPS

SEQ ID NO: 16 - protein, 9 aa, Artificial Sequence
  Synthetic
VVRTPPKSP

SEQ ID NO: 17 - protein, 9 aa, Artificial Sequence
  Synthetic
RTPPKSPSS

SEQ ID NO: 18 - protein, 7 aa, Artificial Sequence
  Synthetic
RTPPKSP

SEQ ID NO: 19 - protein, 7 aa, Artificial Sequence
  Synthetic
PPKSPSS

SEQ ID NO: 20 - protein, 13 aa, Artificial Sequence
  Synthetic
SRSRTPSLPTPPT

SEQ ID NO: 21 - protein, 7 aa, Artificial Sequence
  Synthetic
SRTPSLP

SEQ ID NO: 22 - protein, 7 aa, Artificial Sequence
  Synthetic
RTPSLPT

SEQ ID NO: 23 - protein, 7 aa, Artificial Sequence
  Synthetic
RSRTPSL

SEQ ID NO: 24 - protein, 11 aa, Artificial Sequence
  Synthetic
PGSRSRTPSLP

SEQ ID NO: 25 - protein, 15 aa, Artificial Sequence
  Synthetic
GYSSPGSPGTPGSRS

SEQ ID NO: 26 - protein, 25 aa, Artificial Sequence
  Synthetic
GYSSPGSPGTPGSRSRTPSLPTPPT

SEQ ID NO: 27 - DNA, 24 bp, Artificial Sequence
  Synthetic
tcgtcgttttgtcgttttgtcgtt

SEQ ID NO: 28 - DNA, 21 bp, Artificial Sequence
  Synthetic
tcgtcgtttttcggtcgtttt

SEQ ID NO: 29 - DNA, 21 bp, Artificial Sequence
  Synthetic
tcgtcgtttttcggtgctttt

SEQ ID NO: 30 - protein, 441 aa, Homo sapien
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 31 - protein, 22 aa, Artificial Sequence
  Synthetic
CGGEIVYKSPVVSGDTSPRHLS

SEQ ID NO: 32 - protein, 34 aa, Artificial Sequence
  Synthetic
CGGRENAKAKTDHGAEIVYKSPVVSGDTSPRHLS

SEQ ID NO: 33 - protein, 13 aa, Artificial Sequence
  Synthetic
CGGEIVYKSPVVS

SEQ ID NO: 34 - protein, 10 aa, Artificial Sequence
  Synthetic
CGGGDTSPRH

SEQ ID NO: 35 - protein, 14 aa, Artificial Sequence
  Synthetic
CGGKSPVVSGDTSP

SEQ ID NO: 36 - protein, 10 aa, Artificial Sequence
  Synthetic
CGGEIVYKSP

SEQ ID NO: 37 - protein, 10 aa, Artificial Sequence
  Synthetic
CGGIVYKSPV

SEQ ID NO: 38 - protein, 10 aa, Artificial Sequence
  Synthetic
CGGVYKSPVV

SEQ ID NO: 39 - protein, 10 aa, Artificial Sequence
  Synthetic
CGGYKSPVVS

SEQ ID NO: 40 - protein, 10 aa, Artificial Sequence
  Synthetic
CGGKSPVVSG

SEQ ID NO: 41 - protein, 22 aa, Artificial Sequence
  Synthetic
EIVYKSPVVSGDTSPRHLSGGC

SEQ ID NO: 42 - protein, 20 aa, Artificial Sequence
  Synthetic
CGGKVAVVRTPPKSPSSAKS

SEQ ID NO: 43 - protein, 12 aa, Artificial Sequence
  Synthetic
CGGVRTPPKSPS

SEQ ID NO: 44 - protein, 12 aa, Artificial Sequence
  Synthetic
CGGVVRTPPKSP

SEQ ID NO: 45 - protein, 12 aa, Artificial Sequence
  Synthetic
CGGRTPPKSPSS

SEQ ID NO: 46 - protein, 10 aa, Artificial Sequence
  Synthetic
CGGRTPPKSP

SEQ ID NO: 47 - protein, 10 aa, Artificial Sequence
  Synthetic
CGGPPKSPSS

SEQ ID NO: 48 - protein, 16 aa, Artificial Sequence
  Synthetic
CGGSRSRTPSLPTPPT

SEQ ID NO: 49 - protein, 10 aa, Artificial Sequence
  Synthetic
CGGSRTPSLP

SEQ ID NO: 50 - protein, 10 aa, Artificial Sequence
  Synthetic
CGGRTPSLPT

SEQ ID NO: 51 - protein, 10 aa, Artificial Sequence
  Synthetic
CGGRSRTPSL

SEQ ID NO: 52 - protein, 14 aa, Artificial Sequence
  Synthetic
CGGPGSRSRTPSLP

SEQ ID NO: 53 - protein, 17 aa, Artificial Sequence
  Synthetic
CGGYSSPGSPGTPGSRS

SEQ ID NO: 54 - protein, 27 aa, Artificial Sequence
  Synthetic
CGGYSSPGSPGTPGSRSRTPSLPTPPT

SEQ ID NO: 55 - protein, 22 aa, Artificial Sequence
  Synthetic
CGGEIVYKSPVVSGDTSPRHLS

SEQ ID NO: 56 - protein, 34 aa, Artificial Sequence; identical to SEQ ID NO: 32
  Synthetic

SEQ ID NO: 57 - protein, 13 aa, Artificial Sequence
  Synthetic
CGGEIVYKSPVVS

SEQ ID NO: 58 - protein, 10 aa, Artificial Sequence
  Synthetic
CGGGDTSPRH

SEQ ID NO: 59 - protein, 14 aa, Artificial Sequence
  Synthetic
CGGKSPVVSGDTSP

SEQ ID NO: 60 - protein, 10 aa, Artificial Sequence
  Synthetic
CGGEIVYKSP

SEQ ID NO: 61 - protein, 10 aa, Artificial Sequence
  Synthetic
CGGIVYKSPV

SEQ ID NO: 62 - protein, 10 aa, Artificial Sequence
  Synthetic
CGGVYKSPVV

SEQ ID NO: 63 - protein, 10 aa, Artificial Sequence
  Synthetic
CGGYKSPVVS

SEQ ID NO: 64 - protein, 10 aa, Artificial Sequence
  Synthetic
CGGKSPVVSG

SEQ ID NO: 65 - protein, 20 aa, Artificial Sequence
  Synthetic
CGGKVAVVRTPPKSPSSAKS

SEQ ID NO: 66 - protein, 12 aa, Artificial Sequence
  Synthetic
CGGVRTPPKSPS

SEQ ID NO: 67 - protein, 12 aa, Artificial Sequence
  Synthetic
CGGVVRTPPKSP

SEQ ID NO: 68 - protein, 12 aa, Artificial Sequence
  Synthetic
CGGRTPPKSPSS

SEQ ID NO: 69 - protein, 10 aa, Artificial Sequence
  Synthetic
CGGPPKSPSS

SEQ ID NO: 70 - protein, 16 aa, Artificial Sequence
  Synthetic
CGGSRSRTPSLPTPPT

SEQ ID NO: 71 - protein, 10 aa, Artificial Sequence
  Synthetic
CGGSRTPSLP

SEQ ID NO: 72 - protein, 10 aa, Artificial Sequence
  Synthetic
CGGRTPSLPT

SEQ ID NO: 73 - protein, 10 aa, Artificial Sequence
  Synthetic
CGGRSRTPSL

SEQ ID NO: 74 - protein, 14 aa, Artificial Sequence
  Synthetic
CGGPGSRSRTPSLP

SEQ ID NO: 75 - protein, 17 aa, Artificial Sequence
  Synthetic
CGGYSSPGSPGTPGSRS

SEQ ID NO: 76 - protein, 27 aa, Artificial Sequence
  Synthetic
CGGYSSPGSPGTPGSRSRTPSLPTPPT

SEQ ID NO: 77 - protein, 12 aa, Artificial Sequence
  Synthetic
IPQSLDSWWTSL

SEQ ID NO: 78 - DNA, 16 bp, Artificial Sequence
  Synthetic
gtattaatgactcgag

SEQ ID NO: 79 - protein, 6 aa, Artificial Sequence
  Synthetic
GGGGGC

SEQ ID NO: 80 - protein, 5 aa, Artificial Sequence
  Synthetic
GGGGC

SEQ ID NO: 81 - protein, 4 aa, Artificial Sequence
  Synthetic
GGGC

SEQ ID NO: 82 - protein, 6 aa, Artificial Sequence
  Synthetic
GGGGGK

SEQ ID NO: 83 - protein, 5 aa, Artificial Sequence
  Synthetic
GGGGK

SEQ ID NO: 84 - protein, 4 aa, Artificial Sequence
  Synthetic
GGGK

SEQ ID NO: 85 - protein, 6 aa, Artificial Sequence
  Synthetic
GGGGSC

SEQ ID NO: 86 - protein, 5 aa, Artificial Sequence
  Synthetic
GGGSC

SEQ ID NO: 87 - protein, 4 aa, Artificial Sequence
  Synthetic
GGSC

SEQ ID NO: 88 - protein, 6 aa, Artificial Sequence
  Synthetic
CSGGGG

SEQ ID NO: 89 - protein, 5 aa, Artificial Sequence
  Synthetic
CSGGG

SEQ ID NO: 90 - protein, 4 aa, Artificial Sequence
  Synthetic
CSGG

SEQ ID NO: 91 - protein, 5 aa, Artificial Sequence
  Synthetic
CGGGG

SEQ ID NO: 92 - protein, 4 aa, Artificial Sequence
  Synthetic
CGGG

SEQ ID NO: 93 - protein, 6 aa, Artificial Sequence
  Synthetic
CGGGGG

SEQ ID NO: 94 - protein, 10 aa, Artificial Sequence
  Synthetic
CGDKTHTSPP

SEQ ID NO: 95 - protein, 10 aa, Artificial Sequence
  Synthetic
DKTHTSPPCG

SEQ ID NO: 96 - protein, 17 aa, Artificial Sequence
  Synthetic
CGGPKPSTPPGSSGGAP

SEQ ID NO: 97 - protein, 18 aa, Artificial Sequence
  Synthetic
PKPSTPPGSSGGAPGGCG

SEQ ID NO: 98 - protein, 6 aa, Artificial Sequence
  Synthetic
GCGGGG

SEQ ID NO: 99 - protein, 6 aa, Artificial Sequence
  Synthetic
GGGGCG

SEQ ID NO: 100 - protein, 6 aa, Artificial Sequence
  Synthetic
CGKKGG

SEQ ID NO: 101 - protein, 6 aa, Artificial Sequence
  Synthetic
CGDEGG

SEQ ID NO: 102 - protein, 6 aa, Artificial Sequence
  Synthetic
GGKKGC

SEQ ID NO: 103 - protein, 6 aa, Artificial Sequence
  Synthetic
GGEDGC

SEQ ID NO: 104 - protein, 4 aa, Artificial Sequence
  Synthetic
GGCG

SEQ ID NO: 105 - protein, 7 aa, Artificial Sequence
  Synthetic
AGTYGLG

SEQ ID NO: 106 - protein, 10 aa, Artificial Sequence
  Synthetic
CGGAGTYGLG

SEQ ID NO: 107 - protein, 10 aa, Artificial Sequence
  Synthetic
CGGAGTYGLG

SEQ ID NO: 108 - protein, 7 aa, Artificial Sequence
  Synthetic
DHAGTYG

SEQ ID NO: 109 - protein, 7 aa, Artificial Sequence
  Synthetic
HAGTYGL

SEQ ID NO: 110 - protein, 7 aa, Artificial Sequence
  Synthetic
GTYGLGD

SEQ ID NO: 111 - protein, 7 aa, Artificial Sequence
  Synthetic
TYGLGDR

SEQ ID NO: 112 - protein, 11 aa, Artificial Sequence
  Synthetic
DHAGTYGLGDR

SEQ ID NO: 113 - protein, 10 aa, Artificial Sequence
  Synthetic
CGGDHAGTYG

SEQ ID NO: 114 - protein, 10 aa, Artificial Sequence
  Synthetic
CGGHAGTYGL

SEQ ID NO: 115 - protein, 10 aa, Artificial Sequence
  Synthetic
CGGGTYGLGD

SEQ ID NO: 116 - protein, 10 aa, Artificial Sequence
  Synthetic
CGGTYGLGDR

SEQ ID NO: 117 - protein, 14 aa, Artificial Sequence
  Synthetic
CGGDHAGTYGLGDR

SEQ ID NO: 118 - protein, 10 aa, Artificial Sequence
  Synthetic
CGGDHAGTYG

SEQ ID NO: 119 - protein, 10 aa, Artificial Sequence
  Synthetic
CGGHAGTYGL

SEQ ID NO: 120 - protein, 10 aa, Artificial Sequence
  Synthetic
CGGGTYGLGD

SEQ ID NO: 121 - protein, 10 aa, Artificial Sequence
  Synthetic
CGGTYGLGDR

SEQ ID NO: 122 - protein, 14 aa, Artificial Sequence
  Synthetic
CGGDHAGTYGLGDR

SEQ ID NO: 123 - protein, 10 aa, Artificial Sequence
  Synthetic
CGGRTPPKSP